(19) United States
(12) Patent Application Publication
(10) Pub. No.: US 2023/0210854 A1
(43) Pub. Date: Jul. 6, 2023

(54) MONO- AND COMBINATION THERAPIES

(71) Applicant: Recurium IP Holdings, LLC, San Diego, CA (US)

(72) Inventors: Ahmed Abdi Samatar, West Windsor, NJ (US); Jiali Li, San Diego, CA (US); Peter Qinhua Huang, San Diego, CA (US); Kevin Duane Bunker, Escondido, CA (US); Fernando Donate, San Diego, CA (US); Brant Clayton Boren, San Diego, CA (US)

(21) Appl. No.: 17/998,561

(22) PCT Filed: May 12, 2021
(86) PCT No.: PCT/US2021/032094

§ 371 Date: Nov. 11, 2022

Related U.S. Application Data
(60) Provisional application No. 63/161,828, filed on Mar. 16, 2021.
(60) Provisional application No. 63/160,325, filed on Mar. 12, 2021.
(60) Provisional application No. 63/089,419, filed on Oct. 8, 2020.
(60) Provisional application No. 63/040,832, filed on Jun. 18, 2020.
(60) Provisional application No. 63/025,490, filed on May 15, 2020.

(51) Int. Cl. A61K 31/519; A61P 35/00; A61P 11/00; A61P 15/00; A61P 15/08; A61P 1/00; A61P 19/08; A61K 31/5025; A61K 31/7068; A61K 31/17; A61K 31/282; A61K 39/395

(52) U.S. Cl. CPC A61K 31/519; A61K 31/17; A61K 31/282; A61K 31/5025; A61K 31/7068; A61K 39/3955; A61P 1/00; A61P 11/00; A61P 15/00; A61P 15/08; A61P 19/08; A61P 35/00

(57) ABSTRACT

Disclosed herein are compounds and combinations of compounds for treating a disease or condition, such as cancer.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Applications Nos. 63/025,490, filed May 15, 2020, 63/040,832, filed Jun. 18, 2020, 63/089,419, filed Oct. 8, 2020, 63/160,325, filed Mar. 12, 2021 and 63/161,828, filed Mar. 16, 2021.

### FIELD

The present application relates to the fields of chemistry, biochemistry and medicine. More particularly, disclosed herein are combination therapies, and methods of treating diseases and/or conditions with a combination therapies descried herein.

### DESCRIPTION

Cancers are a family of diseases that involve abnormal cell growth with the potential to invade or spread to other parts of the body. Cancer treatments today include surgery, hormone therapy, radiation, chemotherapy, immunotherapy, targeted therapy and combinations thereof. Survival rates vary by cancer type and by the stage at which the cancer is diagnosed. In 2019, roughly 1.8 million people will be diagnosed with cancer, and an estimated 606,880 people will die of cancer in the United States. Thus, there still exists a need for effective cancer treatments.

### SUMMARY

Some embodiments described herein relate to a combination of compounds that can include an effective amount of Compound (A), or a pharmaceutically acceptable salt thereof, and an effective amount of one or more of Compound (B), or a pharmaceutically acceptable salt of any of the foregoing.

Some embodiments described herein relate to the use of a combination of compounds for treating a disease or condition, wherein the combination includes an effective amount of Compound (A), or a pharmaceutically acceptable salt thereof, and an effective amount of one or more of Compound (B), or a pharmaceutically acceptable salt of any of the foregoing. Other embodiments described herein relate to the use of a combination of compounds in the manufacture of a medicament for treating a disease or condition, wherein the combination includes an effective amount of Compound (A), or a pharmaceutically acceptable salt thereof, and an effective amount of one or more of Compound (B), or a pharmaceutically acceptable salt of any of the foregoing.

In some embodiments, the disease or condition can be a cancer described herein.

### DRAWINGS

FIG. 1 provides examples of chemotherapeutic agents.

FIG. 2 provides examples of PARP inhibitors.

FIG. 3 provides examples of PD-1 inhibitors.

FIG. 4 provides examples of PD-L1 inhibitors.

FIG. 5 provides examples of Compound (A).

FIG. 6 shows the results of studies of Compound (1A) with or without Talazoparib in the TOV112D cell line.

FIG. 7 shows the results of studies of Compound (1A) with or without Niraparib in the MDA-MB-436 cell line.

FIG. 8 shows the results of studies of Compound (1A) with or without carboplatin a TOV21G xenograft model.

FIG. 9 shows the results of studies of Compound (1A) with or without gemcitabine in a SJSA-1 xenograft model.

FIG. 10 shows the results of studies of Compound (1A) with or without Talazoparib in an OVCAR3 xenograft model.

FIGS. 11-12 show the results of studies of Compound (1A) with or without anti-PD-1 in a MC38 syngeneic tumor model.

FIG. 13 shows the result of an efficacy study of Compound (1A) as a single agent in A427 NSCLC xenograft model.

FIG. 14 shows the results of an efficacy study of Compound (1A) as a single agent in H1755 NSCLC tumor model.

FIG. 15 shows the results of an efficacy study of Compound (1A) as a single agent in SKUT-1 uterine leiomyosarcoma tumor model.

FIG. 16 shows the results of an efficacy study of Compound (1A) as a single agent in OVCAR3 ovarian tumor model.

FIG. 17 shows the results of an efficacy study of Compound (1A) as a single agent in MDA-MB-468 TNBC (Triple negative breast cancer) tumor model.

FIG. 18 shows the results of an efficacy study of Compound (1A) and Niraparib as single agents or in combination in x2 MDA-MB-468 TNBC (Triple negative breast cancer) tumor model.

FIG. 19 shows the results of an efficacy study of Compound (1A) and radiation as single agents or in combination in Fadu Head and Neck tumor model.

FIG. 20 shows inhibition of cell growth by Compound (1A) in combination with hydroxyurea (HU) against UWB 1.289 cells; data is represented by relative light units (RLU).

FIG. 21 shows inhibition of cell growth by Compound (1A) in combination with hydroxyurea (HU) against UWB 1.289 cells. Data is represented as relative light units (RLU) normalized for each hydroxyurea concentration to show the synergistic effects of the combination with Compound (1A) with HU.

FIG. 22 shows inhibition of cell growth by Compound (1A) in combination with hydroxyurea (HU) against OVCAR3 cells; data is represented by relative light units (RLU).

FIG. 23 shows inhibition of cell growth by Compound (1A) in combination with hydroxyurea (HU) against OVCAR3 cells. Data are represented as relative light units (RLU) normalized for each hydroxyurea concentration to show the synergistic effects of the combination with Compound (1A) with HU.

FIG. 24 illustrates that the combination of Compound (1A) and Gemcitabine in a KMS-12-BM cell line.

FIG. 25 illustrates that the combination of Compound (1A) and Gemcitabine in OPM-2 cell line.

FIG. 26 illustrates that the combination of Compound (1A) and Gemcitabine in MOLP-8 cell line.

FIG. 27 shows the results of suboptimal doses of Compound (1A) and Triapine as single agents and in combination in A427 cell growth study.

FIG. 28 shows the results of an efficacy study of Compound (1A) in combination with Doxorubicin in OVCAR3 ovarian tumor model.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) (such as 1, 2 or 3 groups) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, hydroxyalkyl, haloalkoxy, an amino, a mono-substituted amine group, a di-substituted amine group and an amine(C₁-C₆ alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃-, CH₃CH₂-, CH₃CH₂CH₂-, (CH₃)₂CH-, CH₃CH₂CH₂CH₂-, CH₃CH₂CH(CH₃)- and (CH₃)₃C-. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

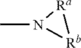

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi- cyclic hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of monocycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro [4.5]decane.

As used herein, “cycloalkenyl” refers to a mono- or multi- cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “carbocyclyl” refers to a non-aromatic a mono- or multicyclic hydrocarbon ring system. When composed of two or more rings, the rings may be joined together in a fused, bridged or spiro fashion, as described herein. Carbocyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A carbocyclyl group may be unsubstituted or substituted. Examples of carbocyclyl groups include, but are in no way limited to, cycloalkyl groups and cycloalkenyl groups, as defined herein, and the non-aromatic portions of 1,2,3,4-tetrahydronaphthalene, 2,3-dihydro-1H-indene, 5,6,7,8-tetrahydroquinoline and 6,7-dihydro-5H-cyclopenta[b]pyridine.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, “lower alkylene groups” are straight-chained —CH₂—tethering groups, forming bonds to connect molecular fragments via their terminal carbon atoms. Examples include but are not limited to methylene (-CH₂-), ethylene (-CH₂CH₂-), propylene (-CH₂CH₂CH₂-) and butylene (-CH₂CH₂CH₂CH₂-). A lower alkylene group can be substituted by replacing one or more hydrogen of the lower alkylene group and/or by substituting both hydrogens on the same carbon with a cycloalkyl group (e.g.,

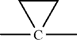

).

As used herein, the term “hydroxy” refers to a —OH group.

As used herein, “alkoxy” refers to the Formula -OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (iso-propoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN″ group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R″ group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(=O)N(R_{A})-” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(=S)N(R_{A})-” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(=O)N(R_{A})-” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “-SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})-” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(=O)O-” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR″ group in which R can be the same as defined with respect to O-carboxy. An ester and C-carboxy may be substituted or unsubstituted.

A “nitro” group refers to an “-NO₂” group.

A “sulfenyl” group refers to an “-SR″ group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R″ group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, trihaloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri- haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The term “amino” as used herein refers to a —NH₂ group.

A “mono-substituted amine” group refers to a “-NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. Examples of mono-substituted amino groups include, but are not limited to, -NH(methyl), -NH(phenyl) and the like.

A “di-substituted amine” group refers to a “-NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. Examples of di-substituted amino groups include, but are not limited to, -N(methyl)₂, -N(phenyl)(methyl), -N(ethyl)(methyl) and the like.

As used herein, “amine(alkyl)” group refers to an -(alkylene)-NR′R″ radical where R′ and R” are independently hydrogen or alkyl as defined herein. An amine(alkyl) may be substituted or unsubstituted. Examples of amine(alkyl) groups include, but are not limited to, -CH₂NH(methyl), -CH₂NH(phenyl), -CH₂CH₂NH(methyl), -CH₂CH₂NH(phenyl), -CH₂N(methyl)₂, -CH₂N(phenyl)(methyl), -NCH₂(ethyl)(methyl), -CH₂CH₂N(methyl)₂, -CH₂CH₂N(phenyl)(methyl), -NCH₂CH₂(ethyl)(methyl) and the like.

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. Those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to the use of a combination of compounds for treating a disease or condition, wherein the combination can include an effective amount of Compound (A), or a pharmaceutically acceptable salt thereof, and an effective amount of one or more of Compound (B), or a pharmaceutically acceptable salt of any of the foregoing, wherein: the Compound (A) has the structure:

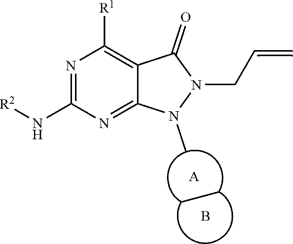

wherein: R¹ can be selected from hydrogen, halogen and a substituted or unsubstituted C₁-C₆ alkyl; Ring A can be selected from a substituted or unsubstituted phenyl and a substituted or unsubstituted 5-6 membered monocyclic heteroaryl; Ring B can be selected from a substituted or unsubstituted monocyclic 5-7 membered carbocyclyl and a substituted or unsubstituted 5-7 membered monocyclic heterocyclyl; R² can be selected from

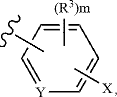

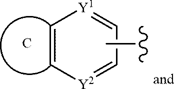

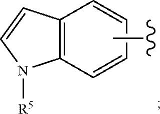

m can be 0, 1, 2 or 3; R³ can be selected from halogen and a substituted or unsubstituted C₁-C₆ alkyl; X can be selected from hydrogen, halogen, hydroxy, cyano, a substituted or unsubstituted 4-6 membered monocyclic heterocyclyl, a substituted or unsubstituted amine(C₁-C₆ alkyl), a substituted or unsubstituted —NH—(CH₂)₁₋₆-amine, a mono-substituted amine, a di-substituted amine, an amino, a substituted or unsubstituted C₁-C₆ alkyl, a substituted or unsubstituted C₁-C₆ alkoxy, a substituted or unsubstituted C₃-C₆ cycloalkoxy, a substituted or unsubstituted (C₁-C₆ alkyl)acyl, a substituted or unsubstituted C-amido, a substituted or unsubstituted N-amido, a substituted or unsubstituted C-carboxy, a substituted or unsubstituted O-carboxy, a substituted or unsubstituted O-carbamyl and a substituted or unsubstituted N-carbamyl; Y can be CH or N; Y¹ can be CR^{4A} or N; Y² can be CR^{4B} or N; Ring C can be selected from a substituted or unsubstituted C₆-C₁₀ aryl, a substituted or unsubstituted monocyclic 5-10 membered heteroaryl, a substituted or unsubstituted monocyclic 5-7 membered carbocyclyl, a substituted or unsubstituted 5-7 membered monocyclic heterocyclyl and a substituted or unsubstituted 7-10 membered bicyclic heterocyclyl; R^{4A} and R^{4B} can be independently selected from hydrogen, halogen and an unsubstituted C₁₋₄ alkyl; and R⁵ can be a substituted or unsubstituted 5-7 membered monocyclic heterocyclyl; and the one or more of Compound (B) can be a PARP inhibitor, a PD-L1 inhibitor and a chemotherapeutic agent, or a pharmaceutically acceptable salt of any of the foregoing.

In some embodiments, R¹ can be selected from hydrogen, halogen and a substituted or unsubstituted C₁-C₆ alkyl. In some embodiments, Ring A can be selected from a substituted or unsubstituted phenyl and a substituted or unsubstituted 5-6 membered monocyclic heteroaryl. In some embodiments, Ring B can be selected from a substituted or unsubstituted monocyclic 5-7 membered carbocyclyl and a substituted or unsubstituted 5-7 membered monocyclic heterocyclyl. In some embodiments, R² can be selected from

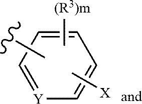

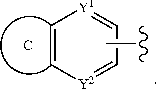

In some embodiments, m can be 0, 1, 2 or 3. In some embodiments, R³ can be selected from halogen and a substituted or unsubstituted C₁-C₆ alkyl. In some embodiments, X can be selected from hydrogen, halogen, hydroxy, cyano, a substituted or unsubstituted 4-6 membered monocyclic heterocyclyl, a substituted or unsubstituted amine(C₁-C₆ alkyl), a substituted or unsubstituted —NH—(CH₂)₁₋₆-amine, a mono-substituted amine, a di-substituted amine, an amino, a substituted or unsubstituted C₁-C₆ alkyl, a substituted or unsubstituted C₁-C₆ alkoxy, a substituted or unsubstituted C₃-C₆ cycloalkoxy, a substituted or unsubstituted (C₁-C₆ alkyl)acyl, a substituted or unsubstituted C-amido, a substituted or unsubstituted N-amido, a substituted or unsubstituted C-carboxy, a substituted or unsubstituted O-carboxy, a substituted or unsubstituted O-carbamyl and a substituted or unsubstituted N-carbamyl. In some embodiments, Y can be CH or N. In some embodiments, Y¹ can be CR^{4A} or N. In some embodiments, Y² can be CR^{4B} or N. In some embodiments, Ring C can be selected from a substituted or unsubstituted C₆-C₁₀ aryl, a substituted or unsubstituted monocyclic 5-10 membered heteroaryl, a substituted or unsubstituted monocyclic 5-7 membered carbocyclyl, a substituted or unsubstituted 5-7 membered monocyclic heterocyclyl and a substituted or unsubstituted 7-10 membered bicyclic heterocyclyl. In some embodiments, R^{4A} and R^{4B} are independently selected from hydrogen, halogen and an unsubstituted C₁₋₄ alkyl.

In some embodiments, R¹ can be selected from hydrogen, halogen and C₁-C₆ alkyl. In some embodiments, R¹ can be hydrogen. In other embodiments, R¹ can be halogen. In some embodiments, R¹ can be fluoro. In still other embodiments, R¹ can be an unsubstituted C₁-C₆ alkyl (such as methyl, ethyl, n-propyl, iso-propyl, n-butyl, iso-butyl, sec-butyl, t-butyl, pentyl (straight chain or branched) or hexyl (straight chain or branched)). In some embodiments, R¹ can be an unsubstituted methyl. In some embodiments, R¹ can be a substituted C₁-C₆ alkyl, such as those described herein. In some embodiments, R¹ can be an unsubstituted C₁-C₆ haloalkyl (such as a C₁-C₆ fluoroalkyl, a C₁-C₆ chloroalkyl or a C₁-C₆ chlorofluoroalkyl). In some embodiments, R¹ can be —CHF₂, —CF₃, —CF₂CH₃ or —CH₂CF₃.

In some embodiments, Ring A can be selected from a substituted or unsubstituted phenyl and a substituted or unsubstituted 5-6 membered monocyclic heteroaryl.

In some embodiments, Ring A can be a substituted phenyl. In other embodiments, Ring A can be an unsubstituted phenyl.

In some embodiments, Ring A can be a substituted 5-6 membered monocyclic heteroaryl. In some embodiments, Ring A can be an unsubstituted 5-6 membered monocyclic heteroaryl. In some embodiments, Ring A can be selected from a substituted or unsubstituted pyrrole, a substituted or unsubstituted furan, a substituted or unsubstituted thiophene, a substituted or unsubstituted imidazole, a substituted or unsubstituted pyrazole, a substituted or unsubstituted oxazole, a substituted or unsubstituted thiazole, a substituted or unsubstituted pyridine, a substituted or unsubstituted pyrazine, a substituted or unsubstituted pyrimidine and a substituted or unsubstituted pyridazine.

When substituted, Ring A can be substituted with one or more substituents selected from halogen, an unsubstituted C₁-C₄ haloalkyl and an unsubstituted C₁-C₄ alkyl. In some embodiments, Ring A is mono-substituted with a halogen (for example, fluoro).

In some embodiments,

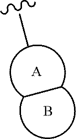

can be selected from:

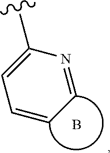

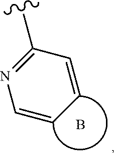

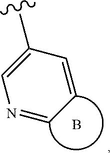

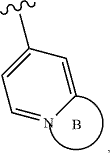

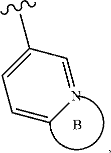

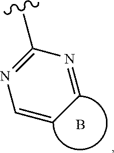

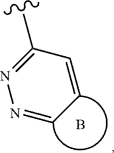

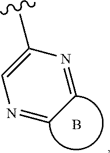

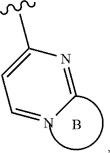

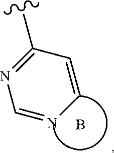

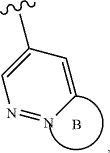

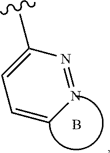

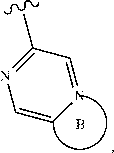

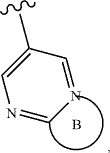

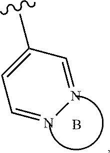

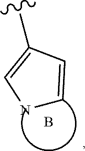

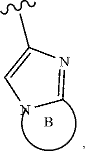

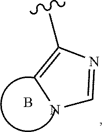

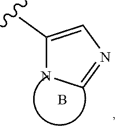

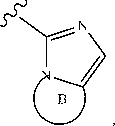

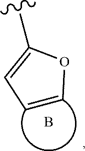

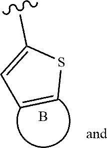

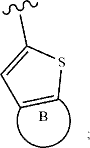

wherein each of the aforementioned groups are substituted or unsubstituted. In some embodiments,

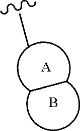

can be a substituted or unsubstituted

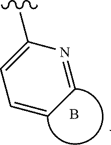

In some embodiments,

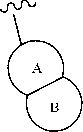

can be a substituted or unsubstituted

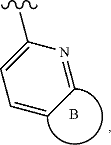

wherein the Ring A is unsubstituted. In other embodiments,

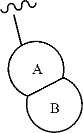

can be selected from a substituted or unsubstituted

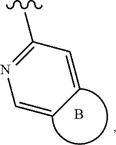

a substituted or unsubstituted

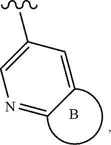

and a substituted or unsubstituted

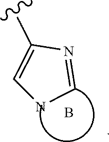

As described herein, the Ring A portion of

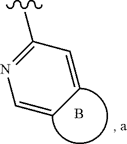

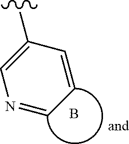

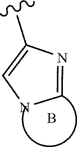

can be unsubstituted.

In some embodiments, Ring B can be selected from a substituted or unsubstituted monocyclic 5-7 membered carbocyclyl and a substituted or unsubstituted 5-7 membered monocyclic heterocyclyl.

In some embodiments, Ring B can be a substituted or unsubstituted monocyclic 5-7 membered carbocyclyl. In some embodiments, Ring B can be a substituted or unsubstituted monocyclic 5 membered carbocyclyl. In other embodiments, Ring B can be a substituted or unsubstituted monocyclic 6 membered carbocyclyl. In still other embodiments, Ring B can be a substituted or unsubstituted monocyclic 7 membered carbocyclyl.

In some embodiments,

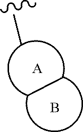

can be selected from:

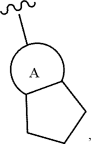

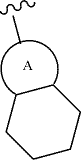

and

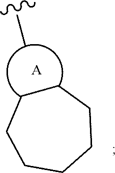

wherein each of the aforementioned groups are substituted or unsubstituted.

In some embodiments, Ring B can be a substituted or unsubstituted monocyclic 5-7 membered heterocyclyl. In some embodiments, Ring B can be a substituted or unsubstituted monocyclic 5 membered heterocyclyl. In other embodiments, Ring B can be a substituted or unsubstituted monocyclic 6 membered heterocyclyl. In still other embodiments, Ring B can be a substituted or unsubstituted monocyclic 7 membered heterocyclyl.

In some embodiments,

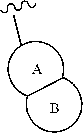

can be selected from:

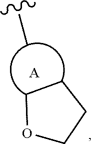

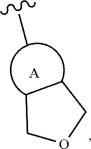

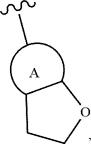

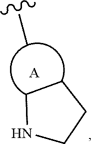

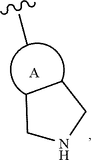

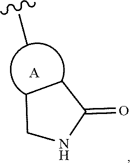

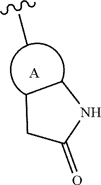

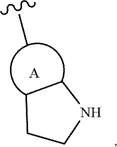

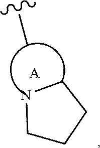

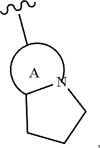

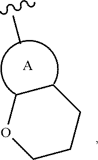

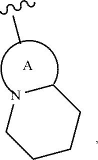

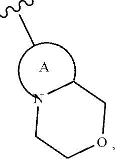

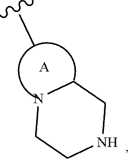

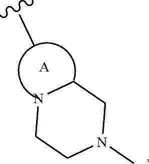

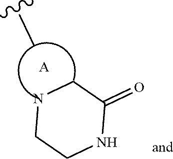

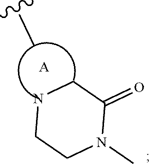

wherein each of the aforementioned groups are substituted or unsubstituted, including any —NH group.

In some embodiments, Ring B can be selected from

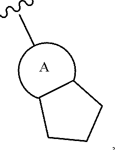

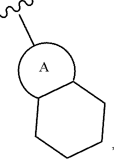

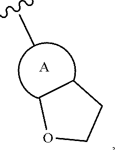

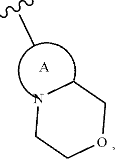

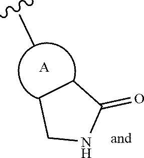

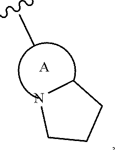

wherein each of the aforementioned groups are substituted or unsubstituted, including any —NH group. In some embodiments, Ring B can be a substituted or unsubstituted

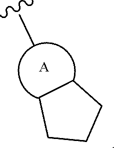

In some embodiments, when Ring B is substituted, Ring B can be substituted with 1, 2 or 3 substituents independently selected from halogen, hydroxy, amino, an unsubstituted N-linked amido (for example, -NHC(O)C₁-C₆ alkyl), an unsubstituted C₁-C₆ haloalkyl (such as those described herein) and a substituted or unsubstituted C₁-C₆ alkyl (such as those described herein). In some embodiments, when Ring B is substituted, Ring B can be substituted with 1, 2 or 3 substituents independently selected from halogen, hydroxy, amino, an unsubstituted N-linked amido (for example, -NHC(O)C₁-C₆ alkyl) and a substituted or unsubstituted C₁-C₆ alkyl (such as those described herein). In some embodiments, Ring B can be substituted with 1, 2 or 3 substituents independently selected from fluoro, hydroxy, amino, an unsubstituted -NHC(O)C₁-C₆ alkyl, an unsubstituted C₁-C₆ haloalkyl (such as those described herein) and an unsubstituted C₁-C₆ alkyl (such as those described herein). In some embodiments, Ring B can be substituted with 1 or 2 substituents independently selected from fluoro, hydroxy, —CF₃, —CHF₂, —CF₂CH₃, an unsubstituted methyl, an unsubstituted ethyl and —NHC(O)CH₃.

In some embodiments,

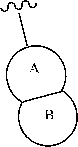

can be selected from:

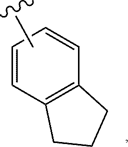

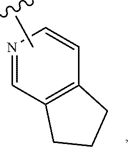

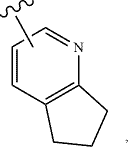

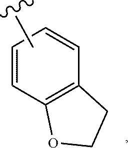

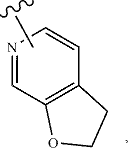

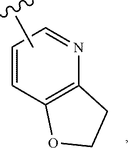

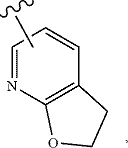

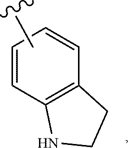

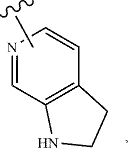

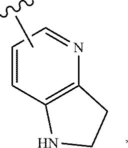

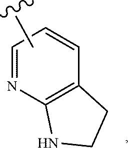

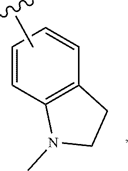

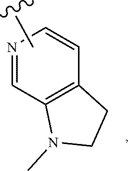

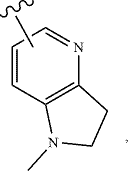

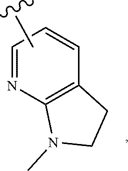

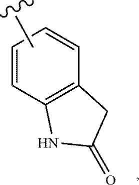

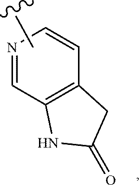

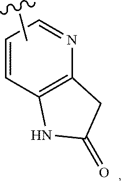

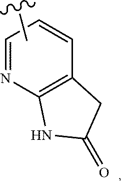

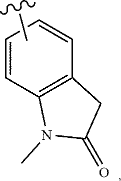

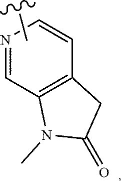

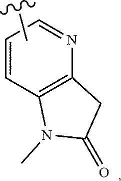

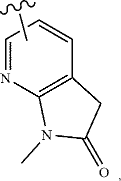

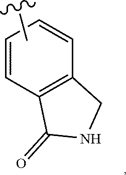

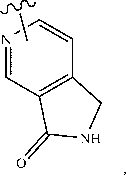

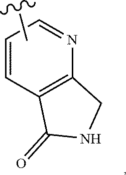

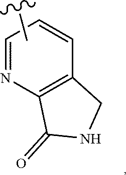

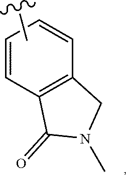

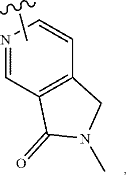

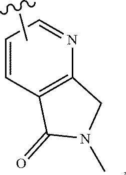

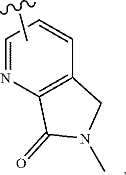

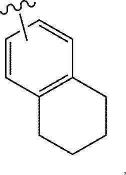

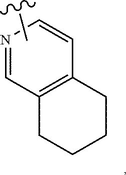

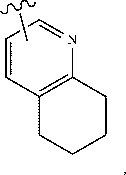

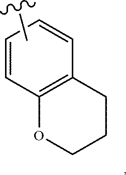

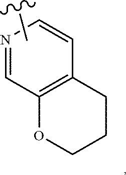

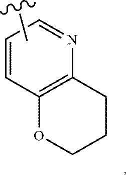

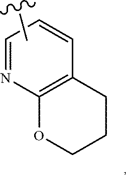

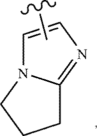

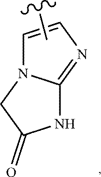

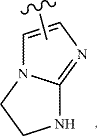

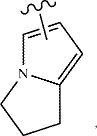

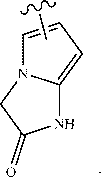

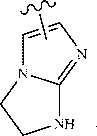

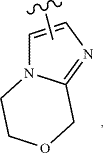

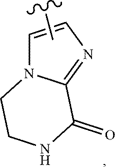

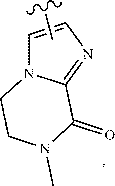

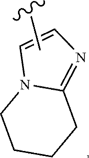

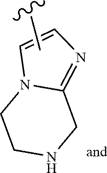

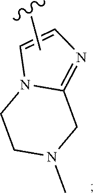

wherein each of the aforementioned groups are substituted or unsubstituted, including any —NH group.

In some embodiments,

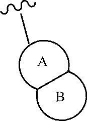

can be selected from:

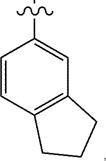

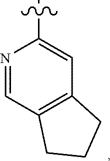

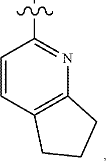

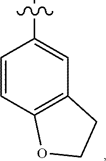

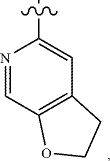

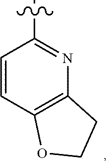

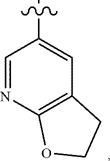

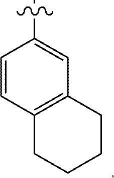

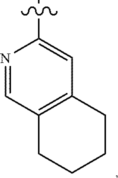

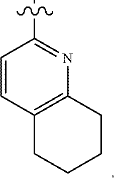

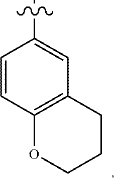

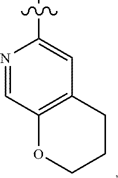

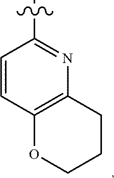

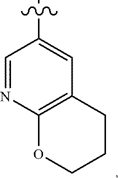

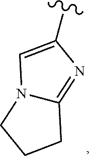

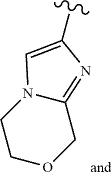

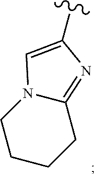

wherein each of the aforementioned groups are substituted or unsubstituted. In some embodiments,

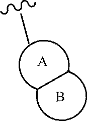

can be selected from:

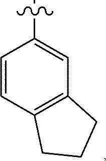

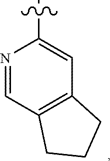

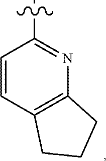

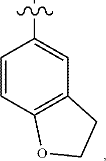

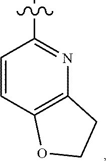

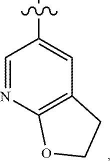

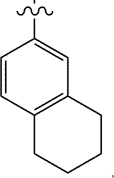

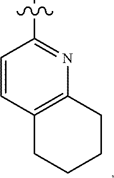

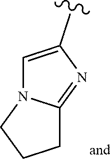

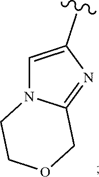

wherein each of the aforementioned groups are substituted or unsubstituted. In some embodiments,

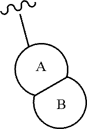

can be a substituted or unsubstituted

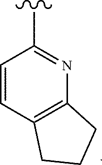

In some embodiments,

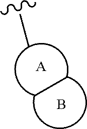

can be a substituted or

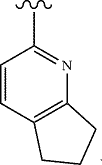

Both Ring A and Ring B can be substituted or unsubstituted. In some embodiments, Ring A and Ring B of

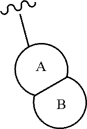

can be independently substituted or unsubstituted. In some embodiments, Ring A and Ring B of

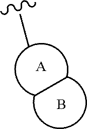

can be both unsubstituted. In some embodiments, Ring A and Ring B of

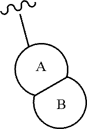

can be both independently substituted. In some embodiments, Ring A of

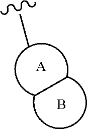

can be substituted and Ring B of

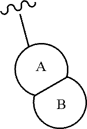

can be unsubstituted. In some embodiments, Ring A of

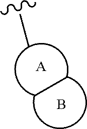

can be unsubstituted and Ring B of

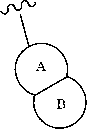

can be substituted. In some embodiments, Ring A of

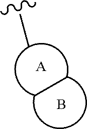

can be unsubstituted and Ring B of

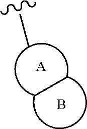

can be substituted with 1, 2 or 3 substituents independently selected from halogen, hydroxy and a substituted or unsubstituted C₁-C₆ alkyl (such as those described herein). In some embodiments, Ring A of

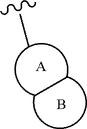

can be unsubstituted and Ring B of

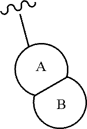

can be substituted with 1, 2 or 3 substituents independently selected from fluoro, hydroxy, amino, an unsubstituted N-linked amido (for example, —NHC(O)C₁-C₆ alkyl), an unsubstituted C₁-C₆ haloalkyl (such as those described herein) and an unsubstituted C₁-C₆ alkyl (such as those described herein). In some embodiments, Ring A of

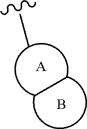

can be unsubstituted and Ring B of

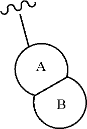

can be substituted with 1 or 2 substituents independently selected from fluoro, hydroxy, amino, —CF₃, —CHF₂, —CF₂CH₃, an unsubstituted methyl, an unsubstituted ethyl and —NHC(O)CH₃.

In some embodiments, R² can be selected from

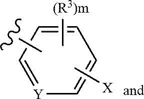

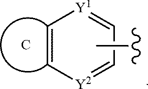

In some embodiments, R² can be

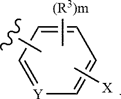

In some embodiments, R² can be

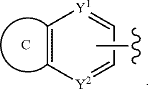

In some embodiments, Y can be CH or N (nitrogen). In some embodiments, Y can be CH. In some embodiments, Y can be N (nitrogen).

In some embodiments, R³ can be selected from halogen and a substituted or unsubstituted C₁-C₆ alkyl (such as those described herein). In some embodiments, R³ can be halogen. In some embodiments, R³ can be a substituted C₁-C₆ alkyl (such as those described herein). In some embodiments, R³ can be an unsubstituted C₁-C₆ alkyl (such as those described herein).

In some embodiments, m can be 0, 1, 2 or 3. In some embodiments, m can be 0. In some embodiments, m can be 1. In some embodiments, m can be 2. In some embodiments, m can be 3. When m is 2 or 3, the R³ groups can be the same or different from each other.

In some embodiments, X can be selected from hydrogen, halogen, hydroxy, cyano, a substituted or unsubstituted 4-6 membered monocyclic heterocyclyl, a substituted or unsubstituted amine(C₁-C₆ alkyl), a substituted or unsubstituted —NH—(CH₂)₁₋₆-amine, a mono-substituted amine, a di-substituted amine, an amino, a substituted or unsubstituted C₁-C₆ alkyl (such as those described herein), a substituted or unsubstituted C₁-C₆ alkoxy (such as methoxy, ethoxy, n-propoxy, iso-propoxy, n-butoxy, iso-butoxy, sec-butoxy, t-butoxy, pentoxy (straight chain or branched) or hexoxy (straight chain or branched)), a substituted or unsubstituted C₃-C₆ cycloalkoxy (such as cyclopropoxy, cyclobutoxy, cyclopentoxy or cyclohexoxy), a substituted or unsubstituted (C₁-C₆ alkyl)acyl, a substituted or unsubstituted C-amido, a substituted or unsubstituted N-amido, a substituted or unsubstituted C-carboxy, a substituted or unsubstituted O-carboxy, a substituted or unsubstituted O-carbamyl and a substituted or unsubstituted N-carbamyl.

In some embodiments, X can be hydrogen. In other embodiments, X can be halogen. In some embodiments, X can be fluoro. In some embodiments, X can be chloro. In still other embodiments, X can be hydroxy. In yet still other embodiments, X can be cyano. In some embodiments, X can be an amino.

In some embodiments, X can be an unsubstituted C₁-C₆ alkyl (such as those described herein). In some embodiments, X can be an unsubstituted methyl, an unsubstituted ethyl or an unsubstituted iso-propyl. In some embodiments, X can be a substituted C₁-C₆ alkyl (such as those described herein). In some embodiments, X can be an unsubstituted C₁-C₆ haloalkyl (such as a C₁-C₆ fluoroalkyl, a C₁-C₆ chloroalkyl or a C₁-C₆ chlorofluoroalkyl). In some embodiments, X can be selected from —CHF₂, —CF₃, —CF₂CH₃ and —CH₂CF₃. In some embodiments, X can be an unsubstituted C₁-C₆ hydroxyalkyl (such as a C₁-C₆ mono-hydroxyalkyl or a C₁-C₆ di-hydroxyalkyl). In some embodiments, X can be selected from —CH₂OH, —CH₂CH₂OH, —CH(OH)CH₃ and —C(OH)(CH₃)₂. In some embodiments, X can be an unsubstituted C₁-C₆ cyanoalkyl (such as a C₁-C₆ mono-cyanoalkyl or a C₁-C₆ di-cyanoalkyl). In some embodiments, X can be selected from

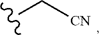

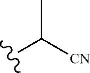

and

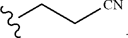

In some embodiments, X can be an unsubstituted C₁-C₆ alkoxyalkyl (such as a C₁-C₆ mono-alkoxyalkyl or a C₁-C₆ di-alkoxyalkyl). In some embodiments, X can be selected from

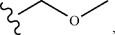

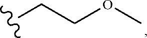

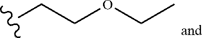

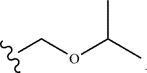

In some embodiments, X can be a substituted C₁-C₆ alkyl selected from

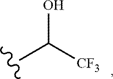

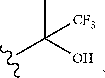

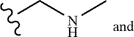

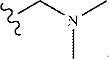

In some embodiments, X can be an unsubstituted C₁-C₆ alkoxy (such as those described herein). In some embodiments, X can be an unsubstituted methoxy, an unsubstituted ethoxy or an unsubstituted iso-propoxy. In some embodiments, X can be a substituted C₁-C₆ alkoxy (such as those described herein). In some embodiments, X can be a C₁-C₆ alkoxy substituted with 1, 2 or 3 substituents independently selected from halogen, an amino, a mono-substituted amine (such as those described herein) and a di-substituted amine (such as those described herein). In some embodiments, X can be a C₁-C₆ alkoxy substituted with 1 substituent selected from halogen, an amino, a mono-substituted amine (such as those described herein) and a di-substituted amine (such as those described herein).

In some embodiments, X can be selected from

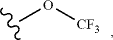

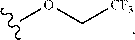

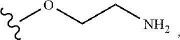

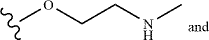

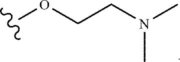

In some embodiments, X can be a substituted C₃-C₆ cycloalkoxy (such as those described herein). In some embodiments, X can be an unsubstituted C₃-C₆ cycloalkoxy (such as those described herein).

In some embodiments, X can be a substituted (C₁-C₆ alkyl)acyl, such as a substituted —(CO)—CH₃. In some embodiments, X can be an unsubstituted (C₁-C₆ alkyl)acyl, such as an unsubstituted -(CO)-CH₃.

In some embodiments, X can be a substituted 4-6 membered monocyclic heterocyclyl. In some embodiments, X can be an unsubstituted 4-6 membered monocyclic heterocyclyl. In some embodiments, X can be selected from azetidine, oxetane, diazetidine, azaoxetane, pyrrolidine, tetrahydrofuran, imidazoline, pyrazolidine, piperidine, tetrahydropyran, piperazine, morpholine and dioxane; wherein each of the aforementioned groups are substituted or unsubstituted, including any —NH group.. In some embodiments, X can be selected from

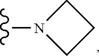

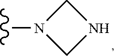

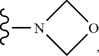

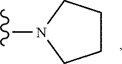

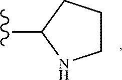

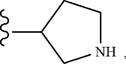

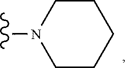

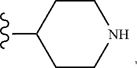

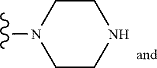

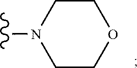

wherein each of the aforementioned groups are substituted or unsubstituted, including any —NH group.

In some embodiments, X can be a 4-6 membered monocyclic heterocyclyl (such as those described herein) substituted with 1 or 2 substituents independently selected from halogen, a substituted or unsubstituted C₁-C₆ alkyl (such as those described herein), a mono-substituted amine (such as those described herein), a di-substituted amine (such as those described herein), an amino, substituted or unsubstituted amine(C₁-C₆ alkyl) and a substituted or unsubstituted (C₁-C₆ alkyl)acyl. In some embodiments, X can be a 4-6 membered monocyclic heterocyclyl substituted with 1 or 2 substituents independently selected from fluoro, an unsubstituted methyl, an unsubstituted ethyl, an unsubstituted iso-propyl, —CH₂OH and —N(CH₃)₂. In some embodiments, X can be selected from

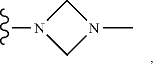

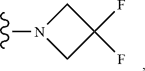

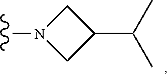

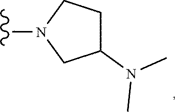

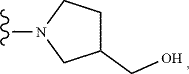

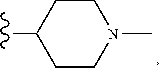

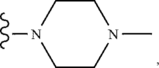

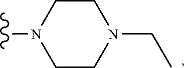

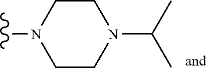

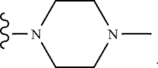

In some embodiments, X can be a substituted amine(C₁-C₆ alkyl). In some embodiments, X can be an unsubstituted amine(C₁-C₆ alkyl). In some embodiments, X can be selected from

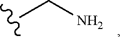

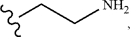

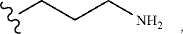

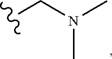

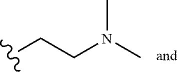

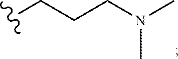

wherein each of the aforementioned groups are substituted or unsubstituted, including any —NH group..

In some embodiments, X can be a substituted —NH—(CH₂)₁₋₆-amine. In some embodiments, X can be an unsubstituted —NH—(CH₂)₁₋₆-amine. In some embodiments, X can be selected from

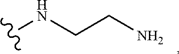

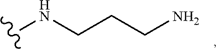

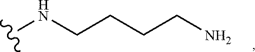

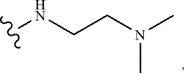

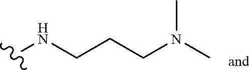

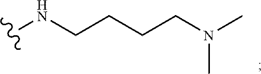

wherein each of the aforementioned groups are substituted or unsubstituted, including any —NH group.

In some embodiments, X can be a mono-substituted amine. In some embodiments, the substituent of the mono-substituted amine is an unsubstituted C₁-C₆ alkyl (such as those as described herein) or an unsubstituted C₃-C₆ cycloalkyl (such as cyclopropyl, cyclobutyl, cyclopentyl and cyclohexyl).

In some embodiments, X can be a di-substituted amine. In some embodiments, the two substituents of the di-substituted amine are independently selected from an unsubstituted C₁-C₆ alkyl (such as those as described herein) and an unsubstituted C₃-C₆ cycloalkyl (such as those as described herein).

In some embodiments, X can be selected from

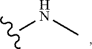

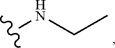

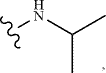

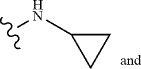

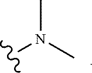

In some embodiments, X can be a substituted or unsubstituted C-amido. In some embodiments, X can be a substituted or unsubstituted N-amido. In some embodiments, X can be a substituted or unsubstituted C-carboxy. In some embodiments, X can be a substituted or unsubstituted O-carboxy. In some embodiments, X can be a substituted or unsubstituted O-carbamyl. In some embodiments, X can be a substituted or unsubstituted N-carbamyl. In some embodiments, X can be mono-substituted with an unsubstituted C₁-C₆ hydroxyalkyl (such as those described herein).

In some embodiments, Y¹ can be CR^{4A} or N (nitrogen). In some embodiments, Y¹ can be CR^{4A}. In some embodiments, Y¹ can be N (nitrogen).

In some embodiments, Y² can be CR^{4B} or N (nitrogen). In some embodiments, Y² can be CR^{4B}. In some embodiments, Y² can be N (nitrogen).

In some embodiments, Y¹ and Y² can each be N (nitrogen). In some embodiments, Y¹ can be CR^{4A} and Y² can be CR^{4B}. In some embodiments, Y¹ can be CR^{4A} and Y² can be N (nitrogen). In some embodiments, Y¹ can be N (nitrogen) and Y² can be CR^{4B}.

In some embodiments, R^{4A} can be hydrogen. In some embodiments, R^{4A} can be halogen. In some embodiments, R^{4A} can be an unsubstituted C₁₋₄ alkyl (such as those described herein).

In some embodiments, R^{4B} can be hydrogen. In some embodiments, R^{4B} can be halogen. In some embodiments, R^{4B} can be an unsubstituted C₁₋₄ alkyl (such as those described herein).

In some embodiments, R^{4A} and R^{4B} can each be hydrogen. In some embodiments, R^{4A} and R^{4B} can each be halogen (wherein the halogens can be the same or different from each other). In some embodiments, R^{4A} and R^{4B} can each be an unsubstituted C₁₋₄ alkyl (such as those described herein, and wherein the C₁₋₄ alkyls can be the same or different from each other). In some embodiments, one of R^{4A} and R^{4B} can be hydrogen and the other of R^{4A} and R^{4B} can be halogen. In some embodiments, one of R^{4A} and R^{4B} can be hydrogen and the other of R^{4A} and R^{4B} can be an unsubstituted C₁₋₄ alkyl (such as those described herein). In some embodiments, one of R^{4A} and R^{4B} can be halogen and the other of R^{4A} and R^{4B} can be an unsubstituted C₁₋₄ alkyl (such as those described herein).

In some embodiments, R² can be

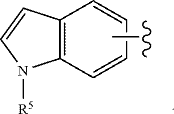

For example, R² can be

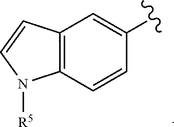

When R² is

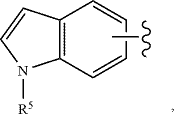

in some embodiments, R⁵ can be a substituted 5-7 membered monocyclic heterocyclyl. In other embodiments, R⁵ can be an unsubstituted 5-7 membered monocyclic heterocyclyl. Examples of R⁵ groups include a substituted or unsubstituted piperidinyl, a substituted or unsubstituted pyrrolidinyl and a substituted or unsubstituted azepanyl. When substituted the R⁵ group, possible substituents include an unsubstituted C₁₋₄ alkyl, halogen, hydroxy and unsubstituted C₁₋₄ haloalkyl.

In some embodiments, Ring C can be selected from a substituted or unsubstituted C₆-C₁₀ aryl, a substituted or unsubstituted monocyclic 5-10 membered heteroaryl, a substituted or unsubstituted monocyclic 5-7 membered carbocyclyl, a substituted or unsubstituted 5-7 membered monocyclic heterocyclyl and a substituted or unsubstituted 7-10 membered bicyclic heterocyclyl.

In some embodiments, Ring C can be a substituted C₆-C₁₀ aryl. In some embodiments, Ring C can be an unsubstituted C₆-C₁₀ aryl. In some embodiments, Ring C can be a substituted C₆ aryl. In some embodiments, Ring C can be an unsubstituted C₆ aryl.

In some embodiments, Ring C can be a substituted 5-10 membered heteroaryl. In some embodiments, Ring C can be an unsubstituted 5-10 membered heteroaryl. In some embodiments, Ring C can be a substituted 5-6 membered heteroaryl. In some embodiments, Ring C can be an unsubstituted 5-6 membered heteroaryl. In some embodiments, Ring C can be selected from furan, thiophene, pyrrole, oxazole, thiazole, imidazole, benzimidazole, indole, pyrazole, isoxazole, pyridine, pyridazine, pyrimidine, pyrazine, purine, quinoline, isoquinoline, quinazoline and quinoxaline; wherein each of the aforementioned groups are substituted or unsubstituted, including any —NH group.

In some embodiments, Ring C can be a substituted or unsubstituted monocyclic 5 membered carbocyclyl. In some embodiments, Ring C can be a substituted or unsubstituted monocyclic 6 membered carbocyclyl. In some embodiments, Ring C can be a substituted or unsubstituted monocyclic 7 membered carbocyclyl.

In some embodiments, Ring C can be a Ring C can be a substituted or unsubstituted 5 membered monocyclic heterocyclyl. In some embodiments, Ring C can be a substituted or unsubstituted 6 membered monocyclic heterocyclyl. In some embodiments, Ring C can be a substituted or unsubstituted 7 membered monocyclic heterocyclyl. In some embodiments, Ring C can be selected from imidazoline, imidazolidine, isoxazoline, isoxazolidine, oxazoline, oxazolidine, oxazolidinone, thiazoline, thiazolidine, morpholine, piperidine, piperazine, pyrrolidine, pyrrolidone, 4-piperidone, pyrazoline, pyrazolidine, tetrahydropyran, azepine, oxepine and diazepine; wherein each of the aforementioned groups are substituted or unsubstituted, including any —NH group.

In some embodiments, Ring C can be a substituted or unsubstituted 7 membered bicyclic heterocyclyl (for example, a fused, a bridged or a spiro heterocyclyl). In some embodiments, Ring C can be a substituted or unsubstituted 8 membered bicyclic heterocyclyl, such as, a fused, a bridged or a spiro heterocyclyl. In some embodiments, Ring C can be a substituted or unsubstituted 9 membered bicyclic heterocyclyl (for example, a fused, a bridged or a spiro heterocyclyl). In some embodiments, Ring C can be a substituted or unsubstituted 10 membered bicyclic heterocyclyl, such as, a fused, a bridged or a spiro heterocyclyl. In some embodiments, Ring C can be selected from pyrrolizidine, indoline, 1,2,3,4 tetrahydroquinoline, 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane; wherein each of the aforementioned groups are substituted or unsubstituted, including any —NH group.

In some embodiments, Ring C can be substituted with one or more substituents independently selected from an unsubstituted C₁-C₆ alkyl (as described herein) and an unsubstituted (C₁-C₆ alkyl)acyl. In some embodiments, Ring C can be substituted with one substituent selected from an unsubstituted C₁-C₆ alkyl (as described herein) and an unsubstituted (C₁-C₆ alkyl)acyl.

In some embodiments, R² can be selected from:

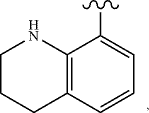

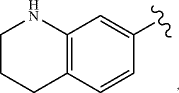

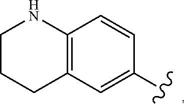

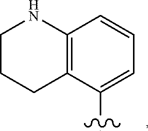

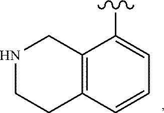

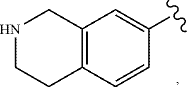

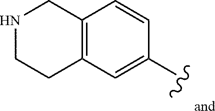

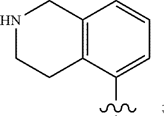

wherein each of the aforementioned groups can be substituted or unsubstituted.

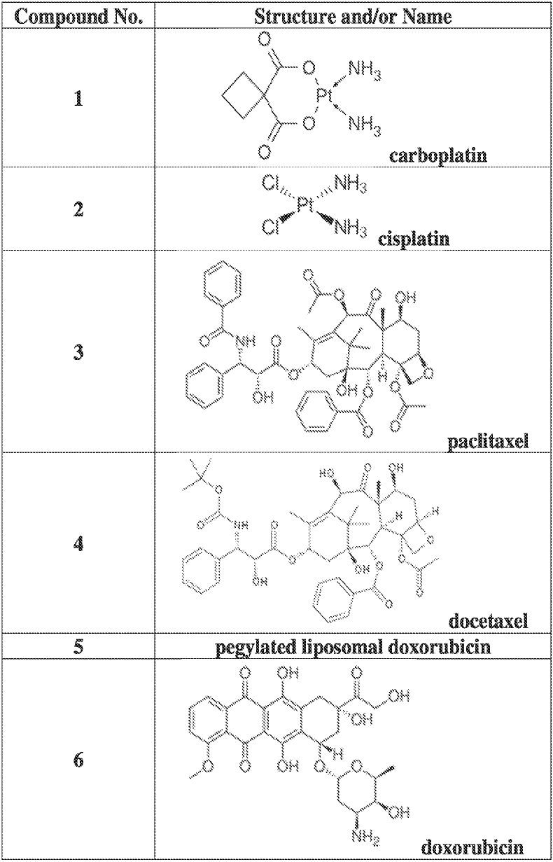

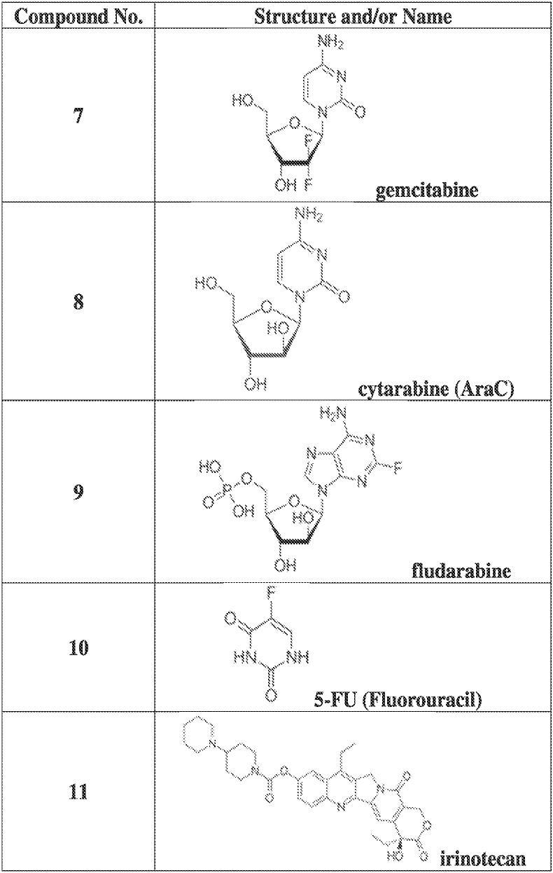

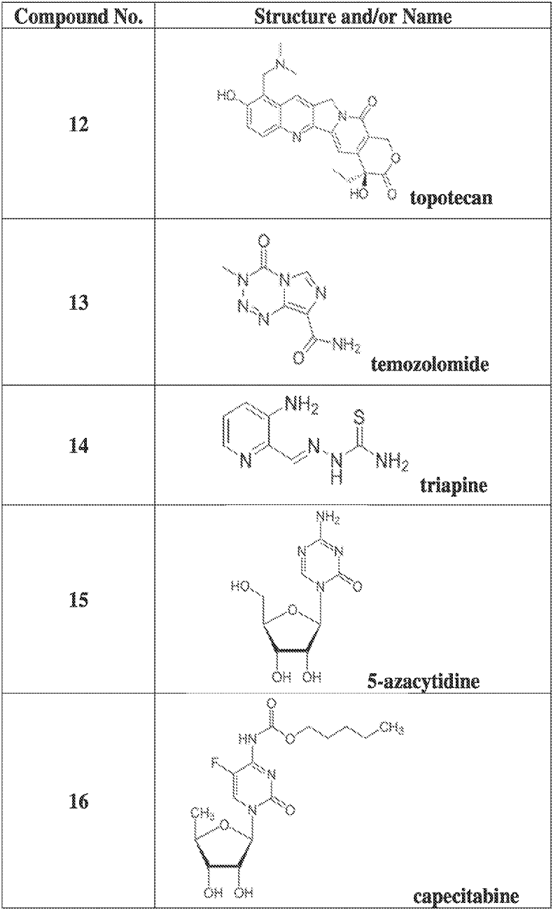

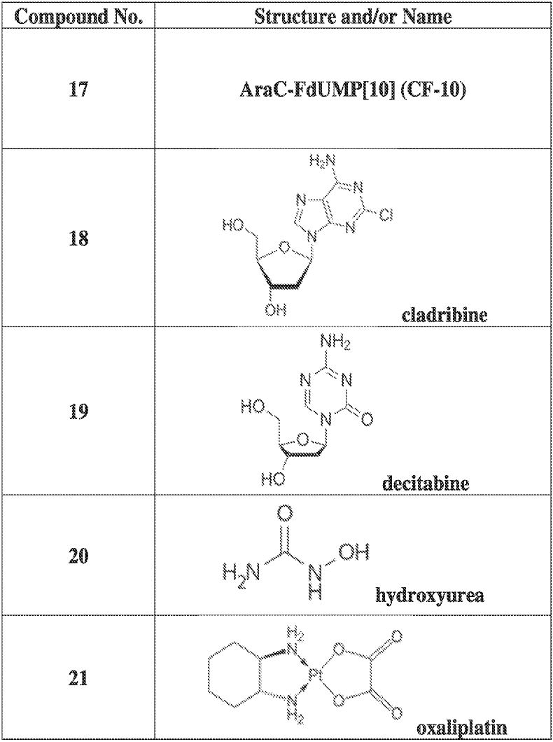

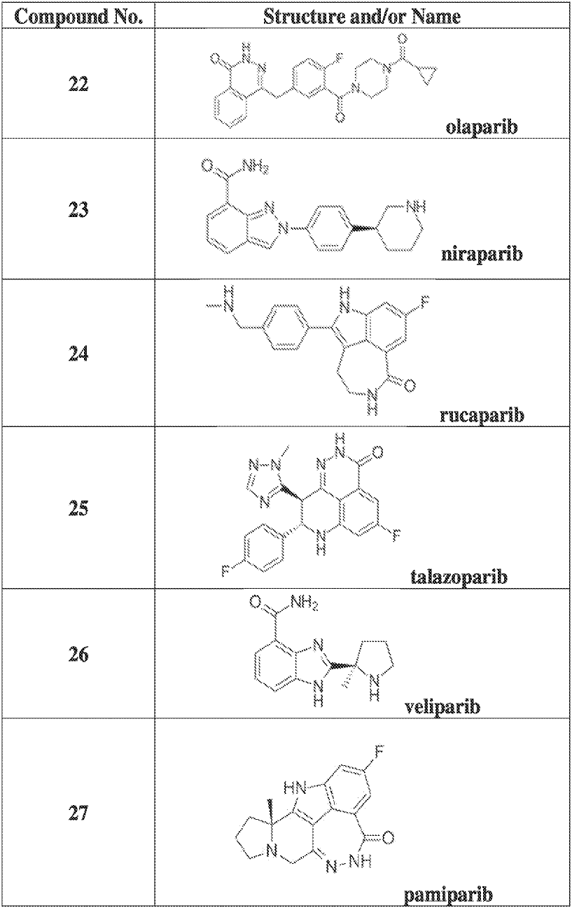

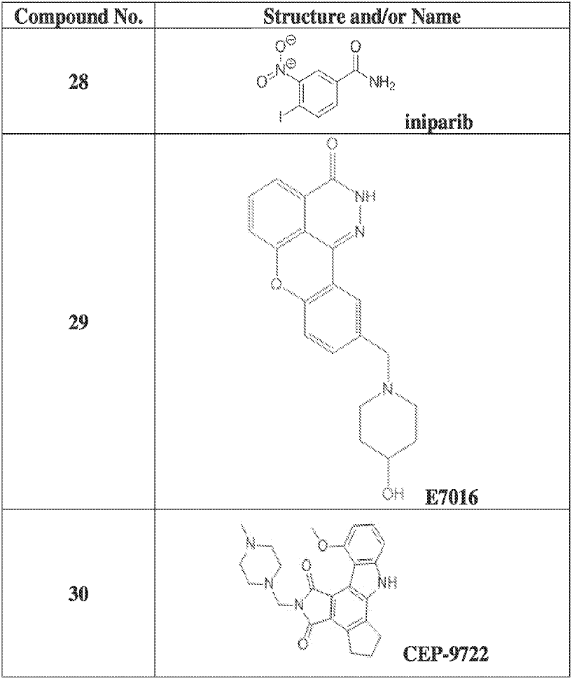

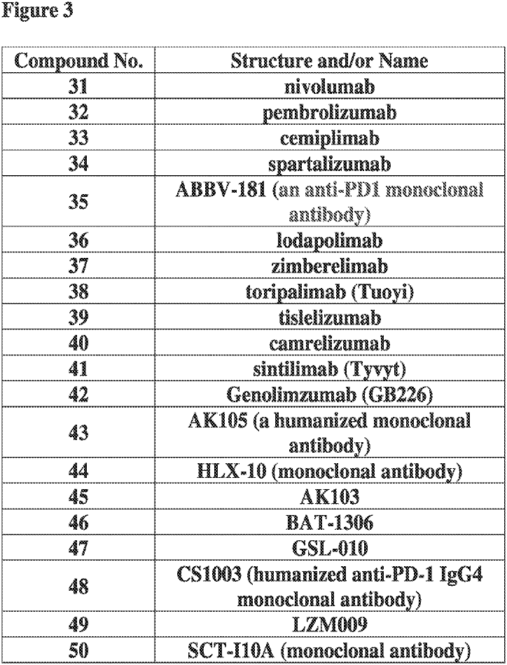

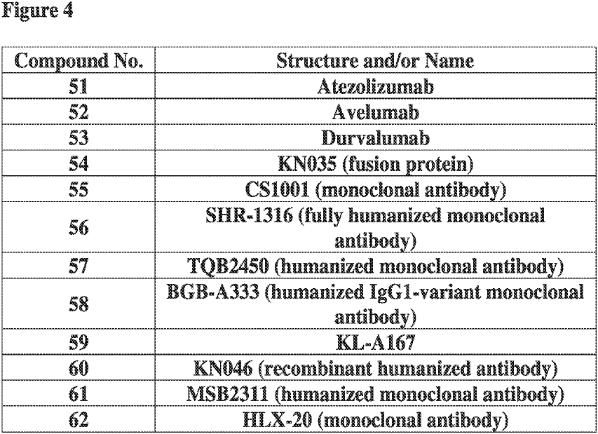

A non-limiting list of chemotherapeutic agents are described herein, and include those provided in FIG. 1. Examples of PARP inhibitors are described herein, and include those provided in FIG. 2. Examples of PD-1 inhibitors are described herein, and include those provided in FIG. 3. Exemplary PD-L1 are described herein, and include those provided in FIG. 4.

Examples of Compound (A) include the following:

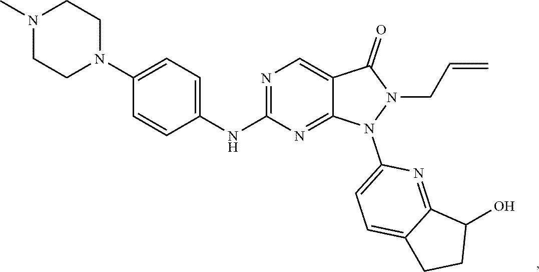

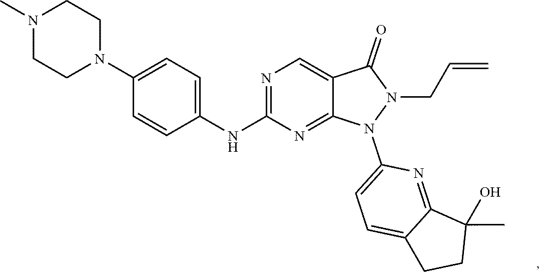

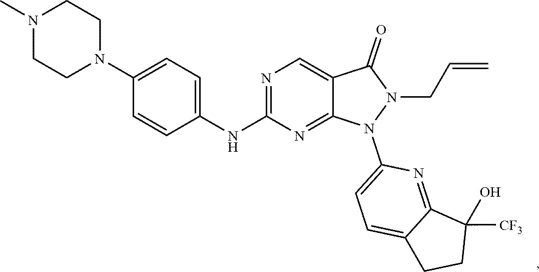

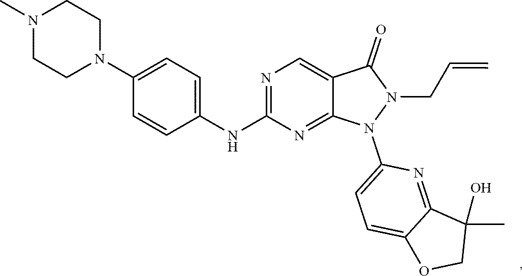

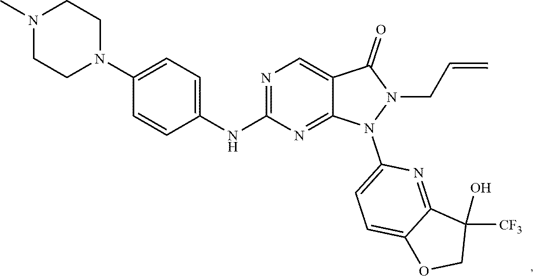

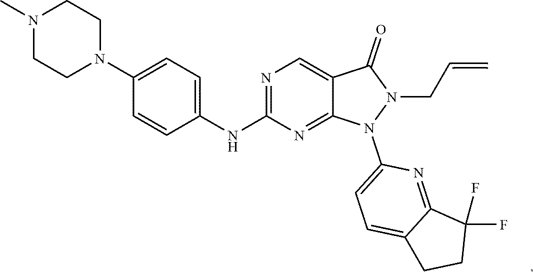

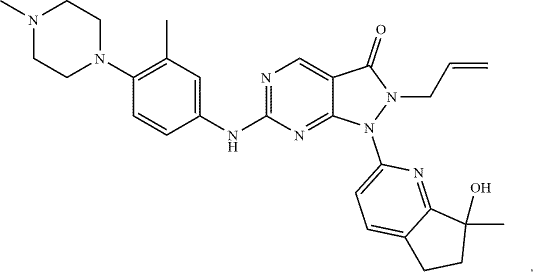

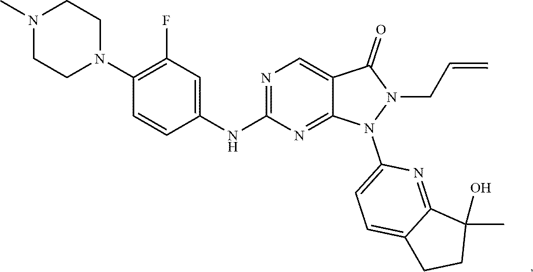

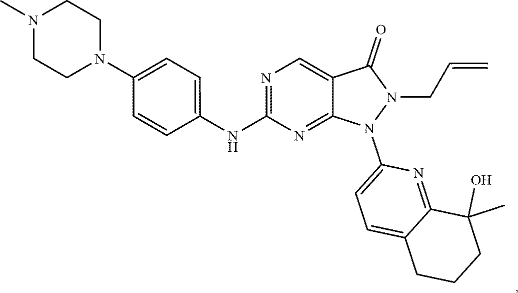

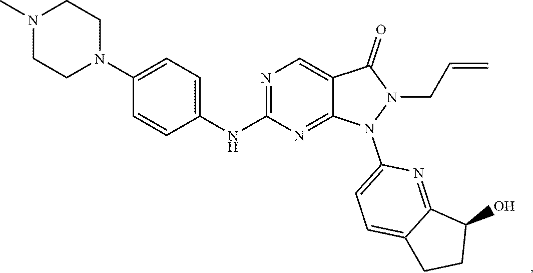

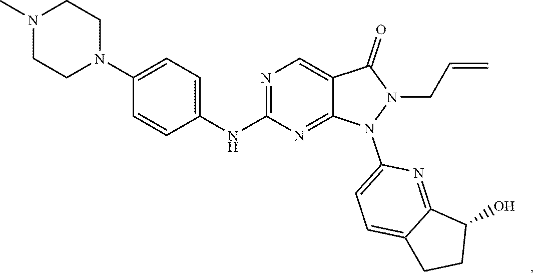

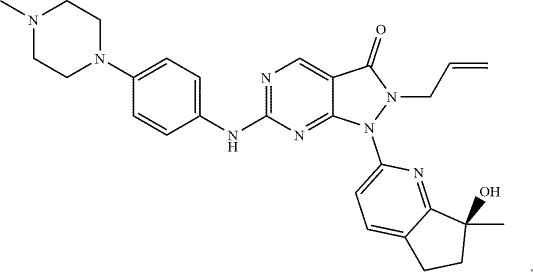

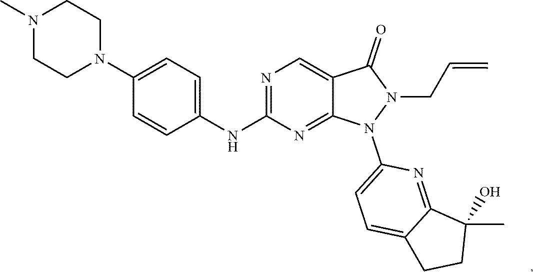

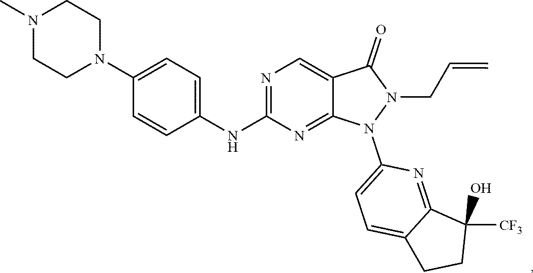

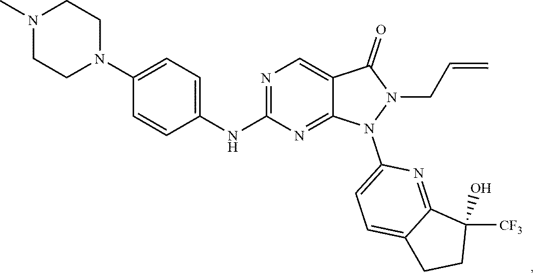

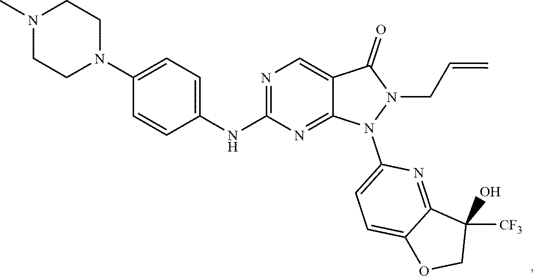

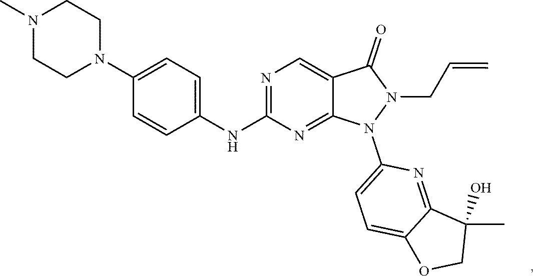

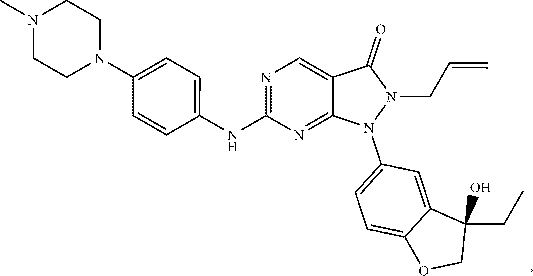

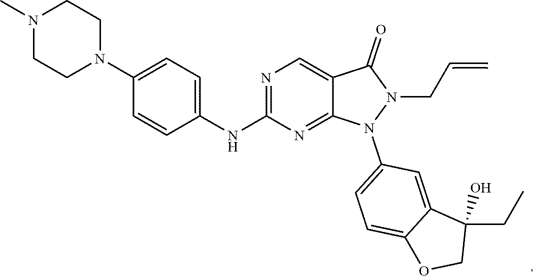

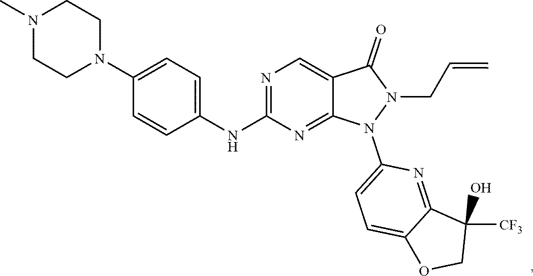

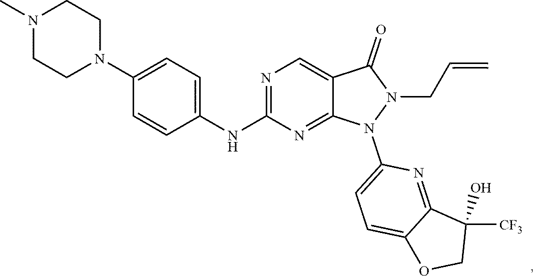

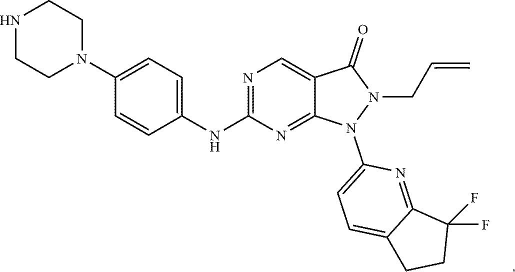

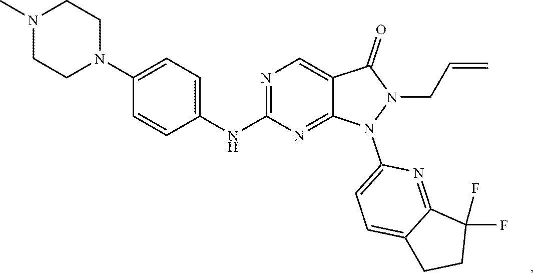

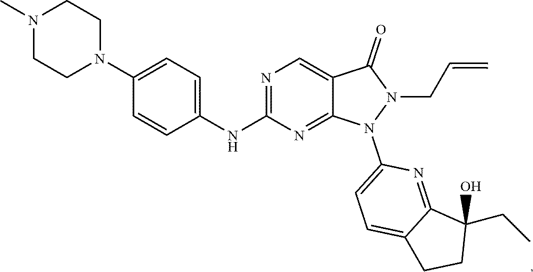

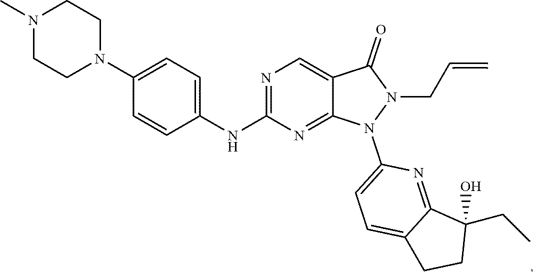

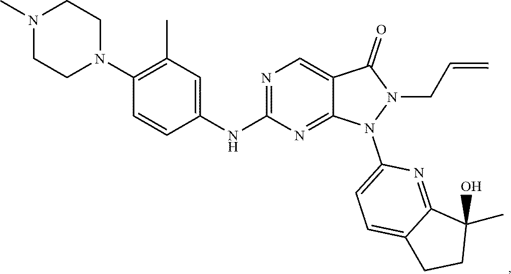

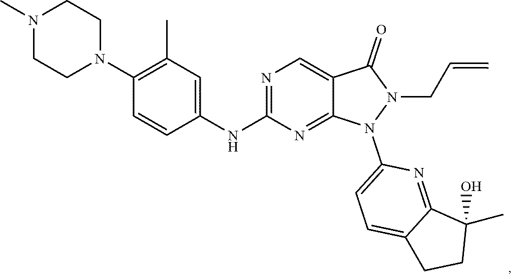

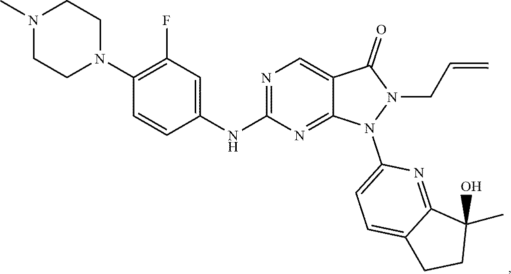

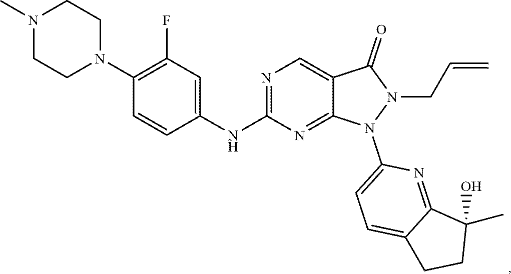

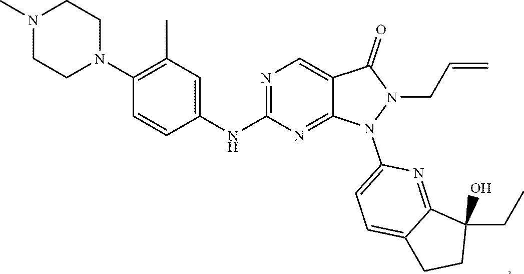

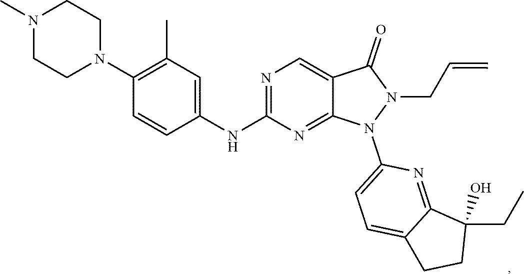

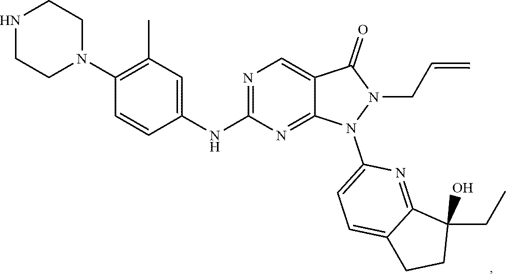

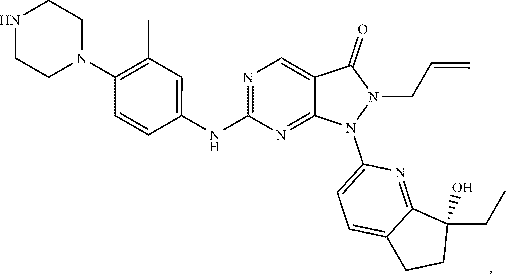

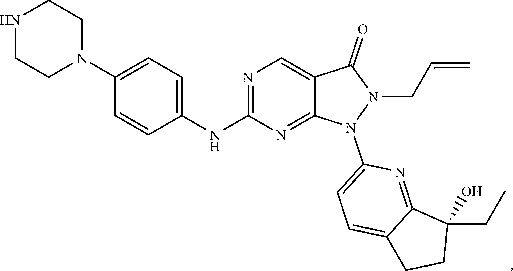

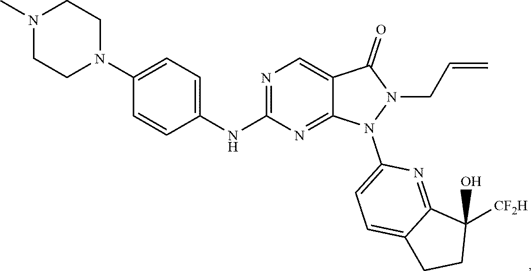

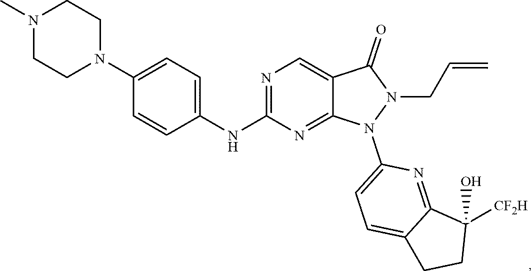

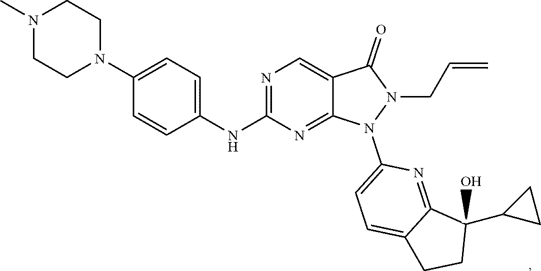

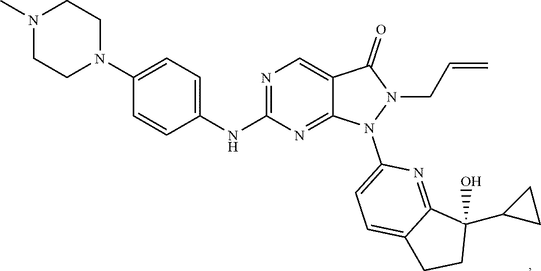

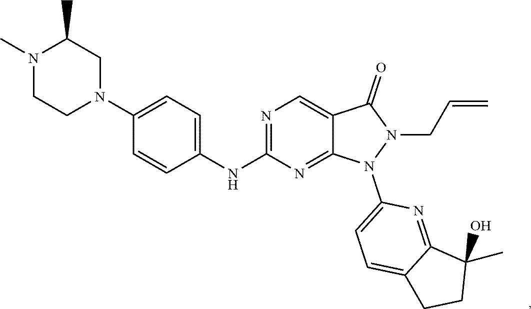

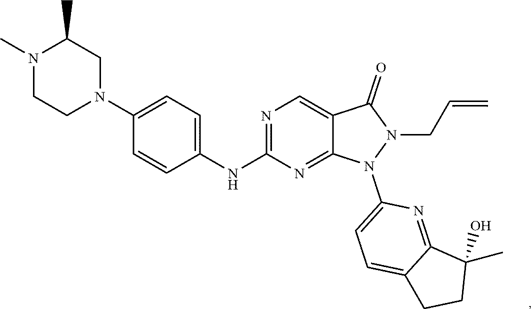

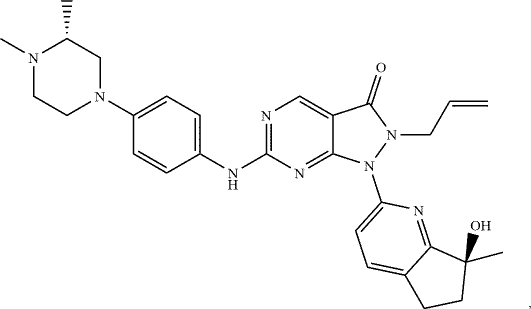

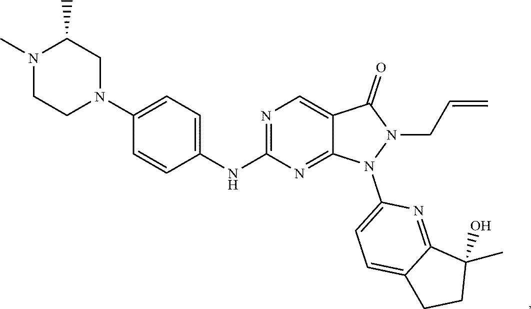

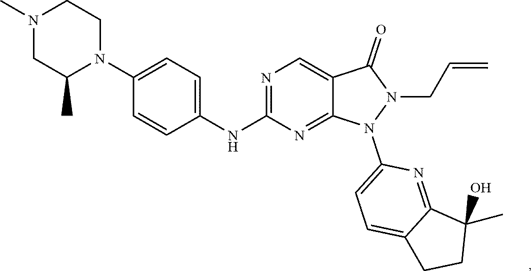

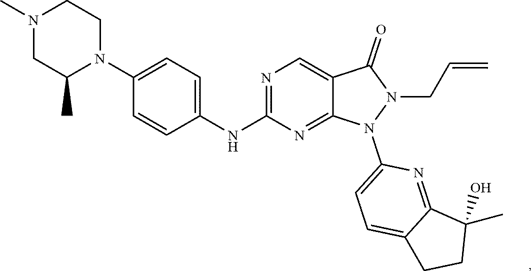

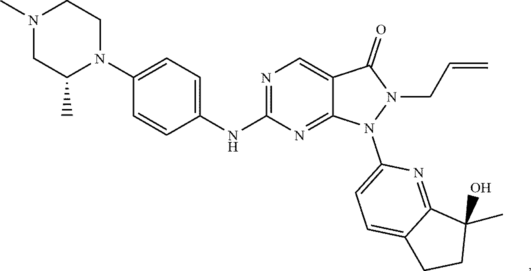

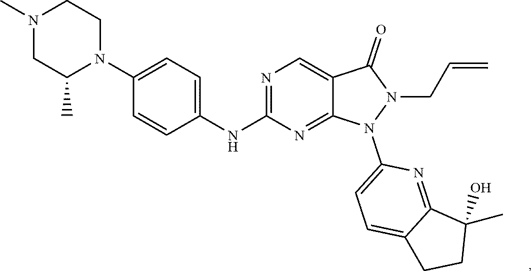

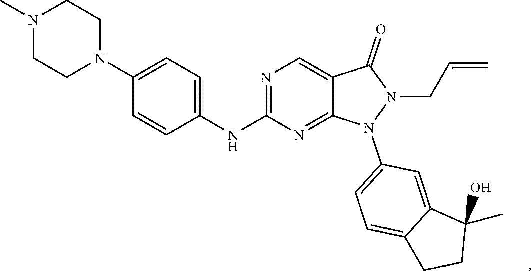

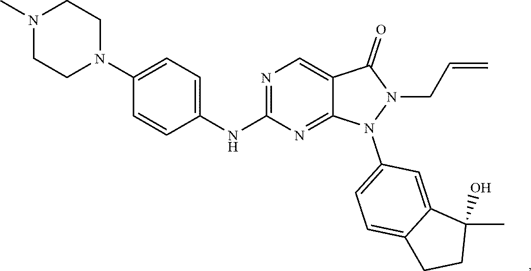

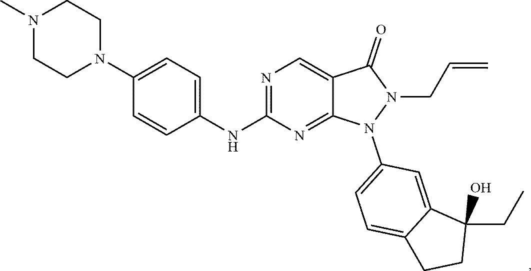

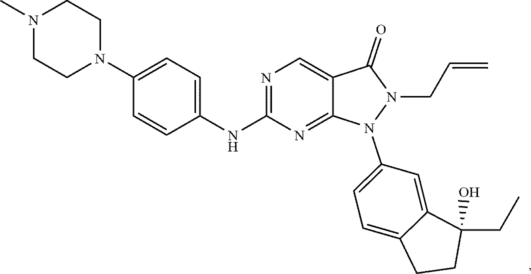

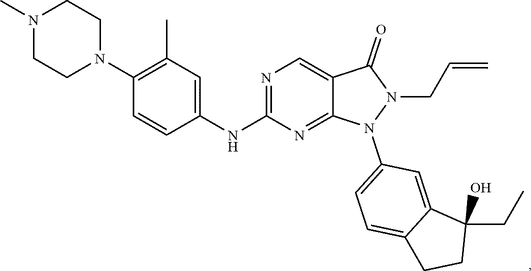

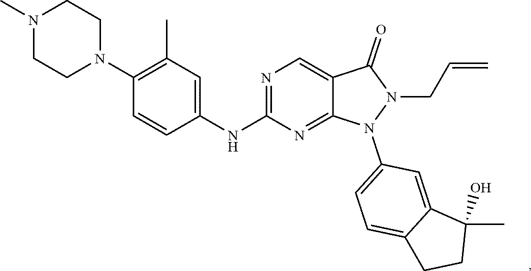

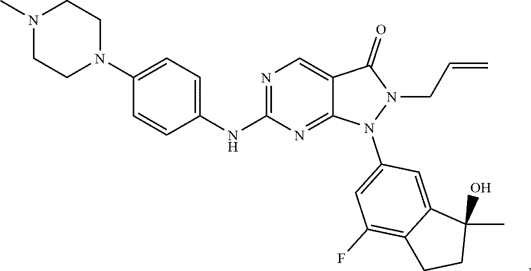

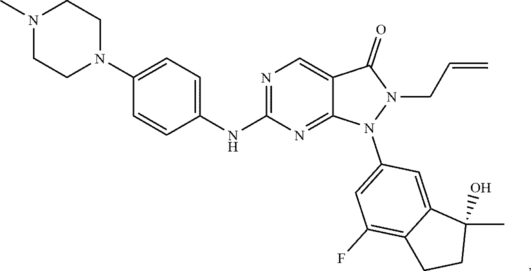

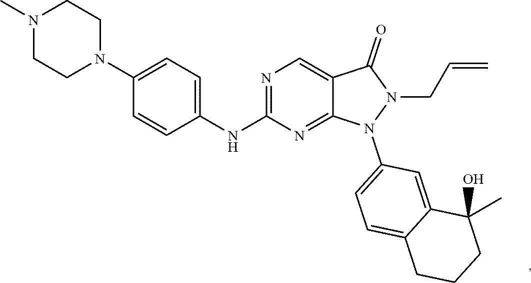

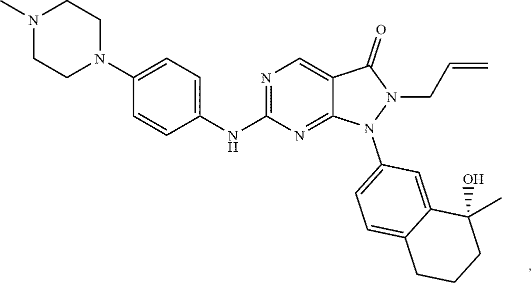

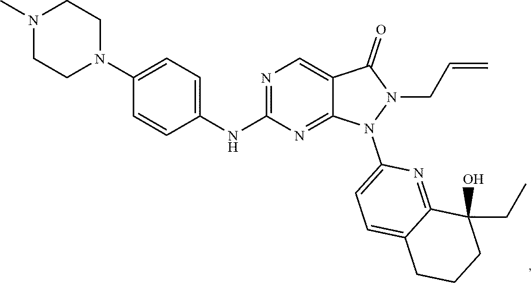

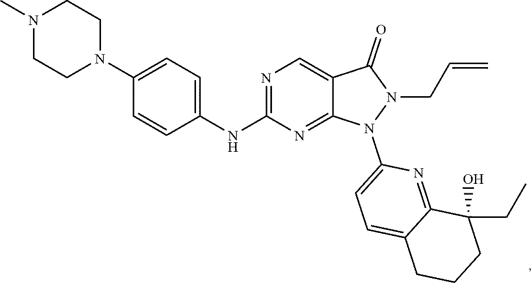

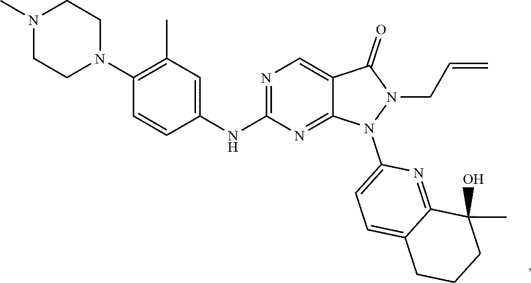

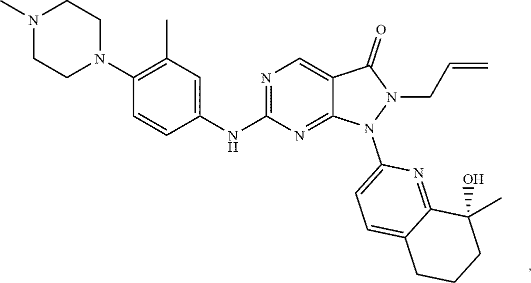

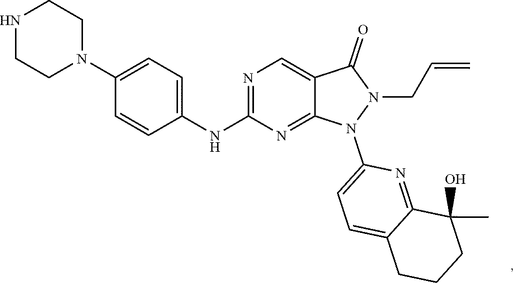

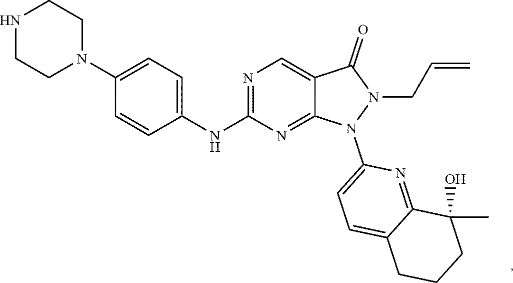

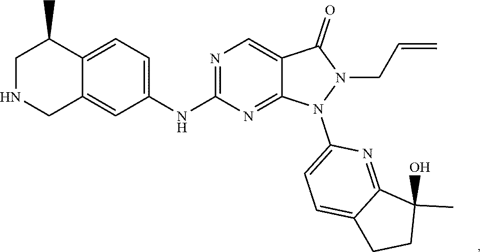

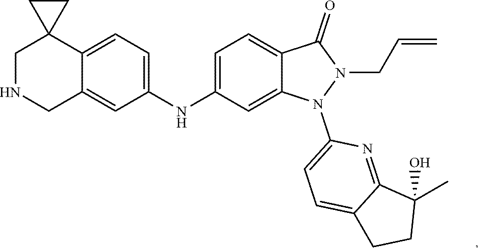

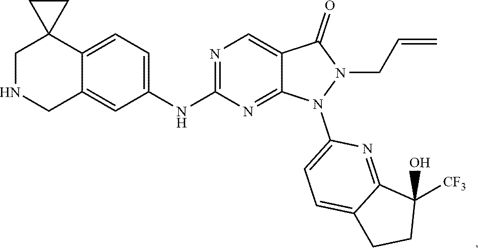

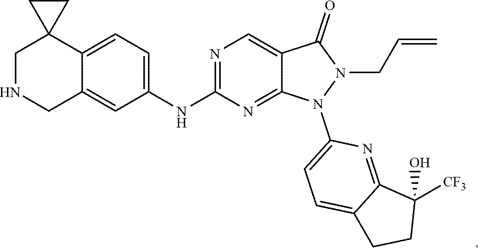

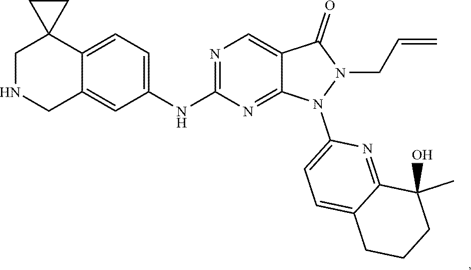

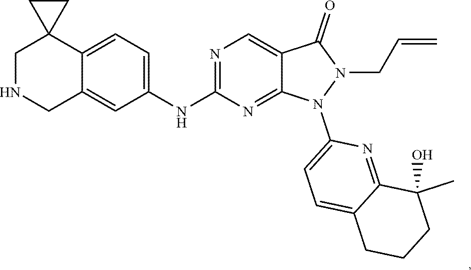

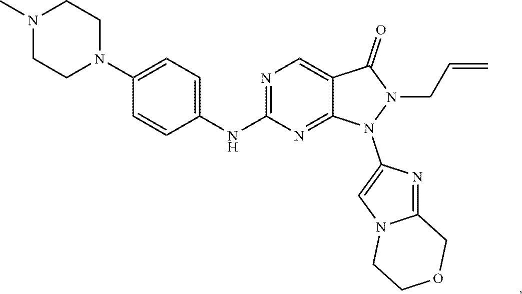

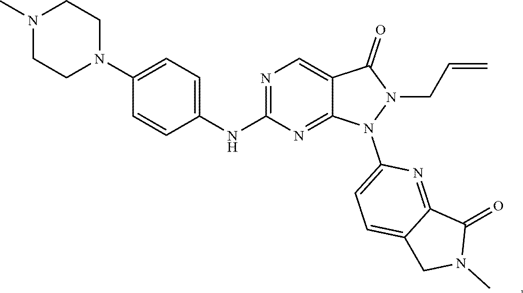

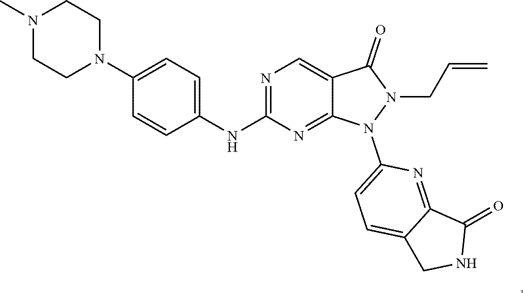

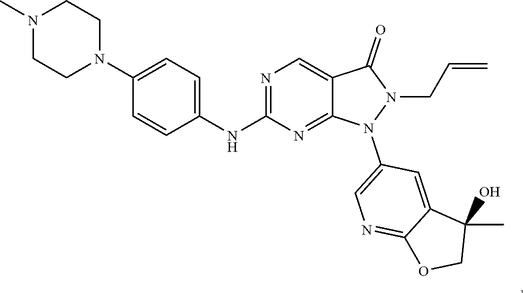

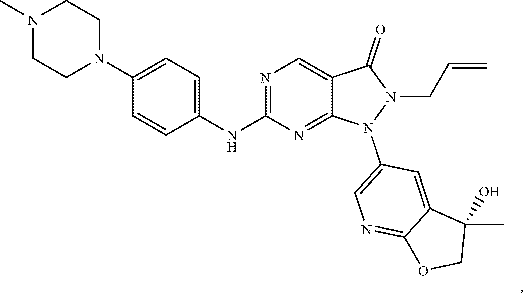

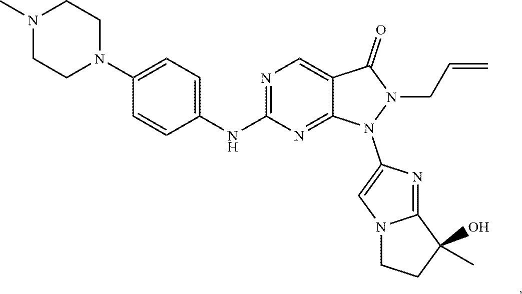

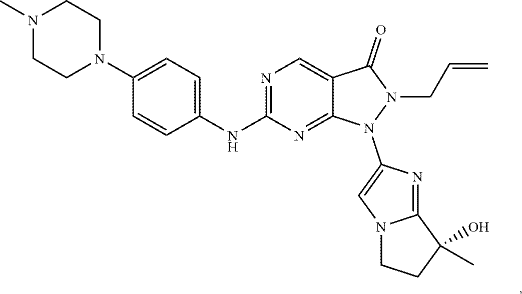

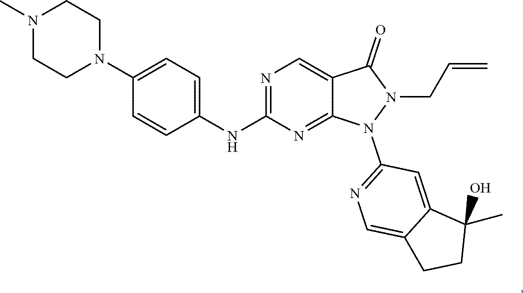

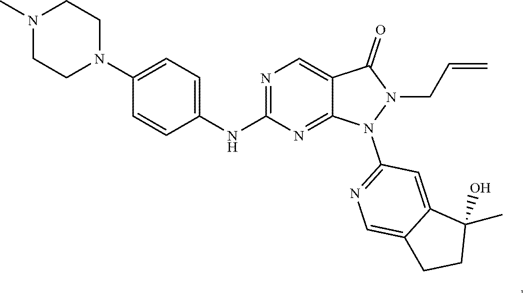

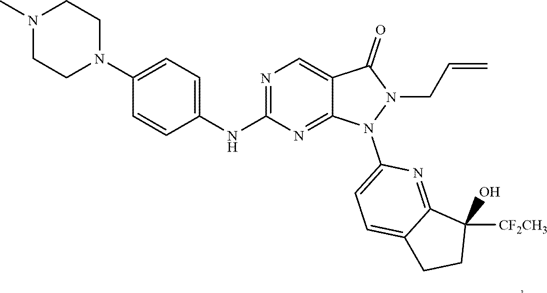

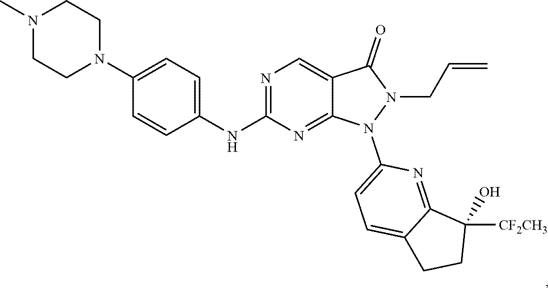

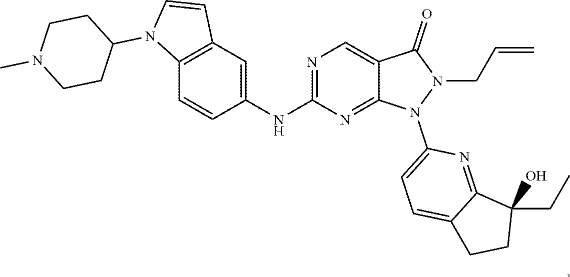

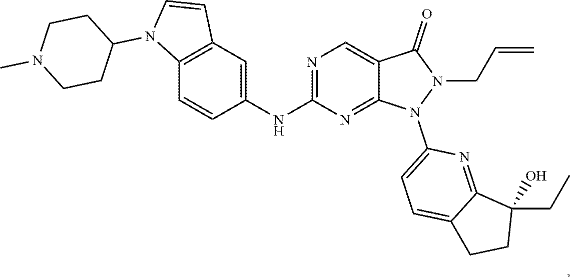

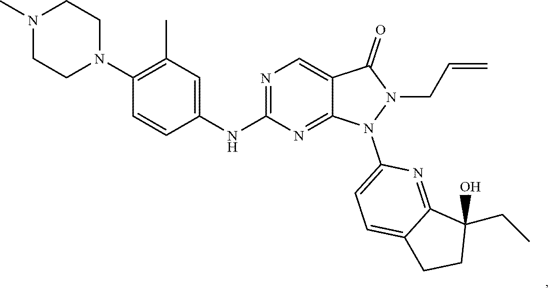

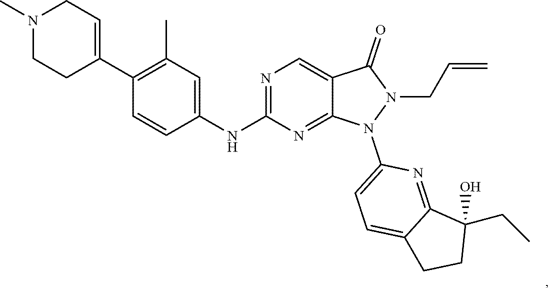

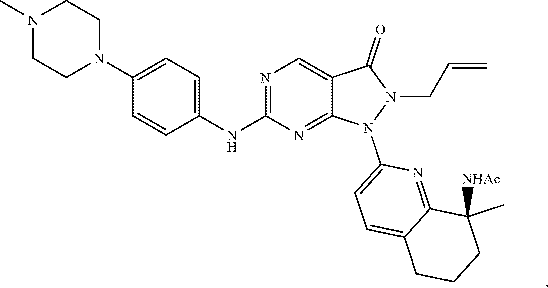

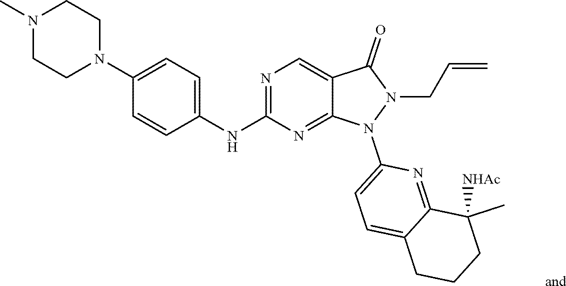

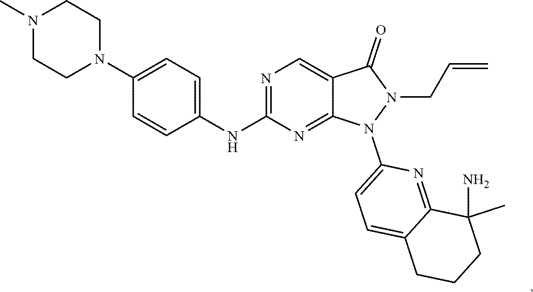

or a pharmaceutically acceptable salt of any of the foregoing.

Compound (A), along with pharmaceutically acceptable salts thereof, can be prepared as described herein and in WO 2019/173082, which is hereby incorporated by reference in its entirety. As described in WO 2019/173082, Compound (A) is a WEE1 inhibitor.

Embodiments of combinations of Compound (A) and Compound (B), including pharmaceutically acceptable salts of the foregoing, are provided in Table 1. For example, in Table 1, a combination represented by 3:5A corresponds to a combination of paclitaxel and

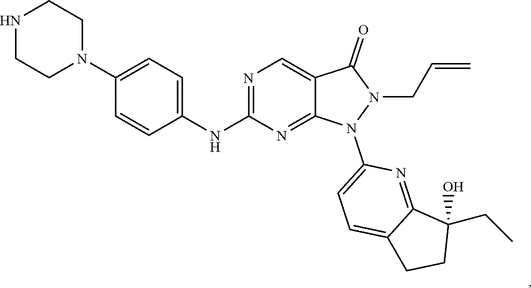

including pharmaceutically acceptable salts of the foregoing. Examples of Compound (A) include those provided in FIG. 5.

TABLE 1

| | | | |
|---|---|---|---|
| Cmpd:Cmpd | Cmpd:Cmpd | Cmpd:Cmpd | Cmpd:Cmpd |
| 1:1A | 9:1A | 17:1A | 25:1A |
| 2:1A | 10:1A | 18:1A | 26:1A |
| 3:1A | 11:1A | 19:1A | 27:1A |
| 4:1A | 12:1A | 20:1A | 28:1A |
| 5:1A | 13:1A | 21:1A | 29:1A |
| 6:1A | 14:1A | 22:1A | 30:1A |
| 7:1A | 15:1A | 23:1A | 31:1A |
| 8:1A | 16:1A | 24:1A | 32:1A |
| 33:1A | 15:2A | 59:2A | 41:3A |
| 34:1A | 16:2A | 60:2A | 42:3A |
| 35:1A | 17:2A | 61:2A | 43:3A |
| 36:1A | 18:2A | 62:2A | 44:3A |
| 37:1A | 19:2A | 1:3A | 45:3A |
| 38:1A | 20:2A | 2:3A | 46:3A |
| 39:1A | 21:2A | 3:3A | 47:3A |
| 40:1A | 22:2A | 4:3A | 48:3A |
| 41:1A | 23:2A | 5:3A | 49:3A |
| 42:1A | 24:2A | 6:3A | 50:3A |
| 43:1A | 25:2A | 7:3A | 51:3A |
| 44:1A | 26:2A | 8:3A | 52:3A |
| 45:1A | 27:2A | 9:3A | 53:3A |
| 46:1A | 28:2A | 10:3A | 54:3A |
| 47:1A | 29:2A | 11:3A | 55:3A |
| 48:1A | 30:2A | 12:3A | 56:3A |
| 49:1A | 31:2A | 13:3A | 57:3A |
| 50:1A | 32:2A | 14:3A | 58:3A |
| 51:1A | 33:2A | 15:3A | 59:3A |
| 52:1A | 34:2A | 16:3A | 60:3A |
| 53:1A | 35:2A | 17:3A | 61:3A |
| 54:1A | 36:2A | 18:3A | 62:3A |
| 55:1A | 37:2A | 19:3A | 1:4A |
| 56:1A | 38:2A | 20:3A | 2:4A |
| 57:1A | 39:2A | 21:3A | 3:4A |
| 58:1A | 40:2A | 22:3A | 4:4A |
| 59:1A | 41:2A | 23:3A | 4:4A |
| 60:1A | 42:2A | 24:3A | 5:4A |
| 61:1A | 43:2A | 25:3A | 6:4A |
| 62:1A | 44:2A | 26:3A | 7:4A |
| 1:2A | 45:2A | 27:3A | 8:4A |
| 2:2A | 46:2A | 28:3A | 9:4A |
| 3:2A | 47:2A | 29:3A | 10:4A |
| 4:2A | 48:2A | 30:3A | 11:4A |
| 5:2A | 49:2A | 31:3A | 12:4A |
| 6:2A | 50:2A | 32:3A | 13:4A |
| 7:2A | 51:2A | 33:3A | 14:4A |
| 8:2A | 52:2A | 34:3A | 15:4A |
| 9:2A | 53:2A | 35:3A | 16:4A |
| 10:2A | 54:2A | 36:3A | 17:4A |
| 11:2A | 55:2A | 37:3A | 18:4A |
| 12:2A | 56:2A | 38:3A | 19:4A |
| 13:2A | 57:2A | 39:3A | 20:4A |
| 14:2A | 58:2A | 40:3A | 21:4A |
| 22:4A | 4:5A | 48:5A | 30:6A |
| 23:4A | 5:5A | 49:5A | 31:6A |
| 24:4A | 6:5A | 50:5A | 32:6A |
| 25:4A | 7:5A | 51:5A | 33:6A |
| 26:4A | 8:5A | 52:5A | 34:6A |
| 27:4A | 9:5A | 53:5A | 35:6A |
| 28:4A | 10:5A | 54:5A | 36:6A |
| 29:4A | 11:5A | 55:5A | 37:6A |
| 30:4A | 12:5A | 56:5A | 38:6A |
| 31:4A | 13:5A | 57:5A | 39:6A |
| 32:4A | 14:5A | 58:5A | 40:6A |
| 33:4A | 15:5A | 59:5A | 41:6A |
| 34:4A | 16:5A | 60:5A | 42:6A |
| 35:4A | 17:5A | 61:5A | 43:6A |
| 36:4A | 18:5A | 62:5A | 44:6A |
| 37:4A | 19:5A | 1:6A | 45:6A |
| 38:4A | 20:5A | 2:6A | 46:6A |
| 39:4A | 21:5A | 3:6A | 47:6A |
| 40:4A | 22:5A | 4:6A | 48:6A |
| 41:4A | 23:5A | 5:6A | 49:6A |
| 42:4A | 24:5A | 6:6A | 50:6A |
| 43:4A | 25:5A | 7:6A | 51:6A |
| 44:4A | 26:5A | 8:6A | 52:6A |
| 45:4A | 27:5A | 9:6A | 53:6A |
| 46:4A | 28:5A | 10:6A | 54:6A |
| 47:4A | 29:5A | 11:6A | 55:6A |
| 48:4A | 30:5A | 12:6A | 56:6A |
| 49:4A | 31:5A | 13:6A | 57:6A |
| 50:4A | 32:5A | 14:6A | 58:6A |
| 51:4A | 33:5A | 15:6A | 59:6A |
| 52:4A | 34:5A | 16:6A | 60:6A |
| 53:4A | 35:5A | 17:6A | 61:6A |
| 54:4A | 36:5A | 18:6A | 62:6A |
| 55:4A | 37:5A | 19:6A | 1:7A |
| 56:4A | 38:5A | 20:6A | 2:7A |
| 57:4A | 39:5A | 21:6A | 3:7A |
| 58:4A | 40:5A | 22:6A | 4:7A |
| 59:4A | 41:5A | 23:6A | 5:7A |
| 60:4A | 42:5A | 24:6A | 6:7A |
| 61:4A | 43:5A | 25:6A | 7:7A |
| 62:4A | 44:5A | 26:6A | 8:7A |
| 1:5A | 45:5A | 27:6A | 9:7A |
| 2:5A | 46:5A | 28:6A | 10:7A |
| 3:5A | 47:5A | 29:6A | 11:7A |
| 12:7A | 55:7A | 36:8A | 18:9A |
| 13:7A | 56:7A | 37:8A | 19:9A |
| 14:7A | 57:7A | 38:8A | 20:9A |
| 15:7A | 58:7A | 39:8A | 21:9A |
| 16:7A | 59:7A | 40:8A | 22:9A |
| 17:7A | 60:7A | 41:8A | 23:9A |
| 18:7A | 61:7A | 42:8A | 24:9A |
| 14:7A | 62:7A | 43:8A | 25:9A |
| 19:7A | 1:8A | 44:8A | 26:9A |
| 20:7A | 2:8A | 45:8A | 27:9A |
| 21:7A | 3:8A | 46:8A | 28:9A |
| 22:7A | 4:8A | 47:8A | 29:9A |
| 23:7A | 5:8A | 48:8A | 30:9A |
| 24:7A | 6:8A | 49:8A | 31:9A |
| 25:7A | 7:8A | 50:8A | 32:9A |
| 26:7A | 8:8A | 51:8A | 33:9A |
| 27:7A | 9:8A | 52:8A | 34:9A |
| 28:7A | 10:8A | 53:8A | 35:9A |
| 29:7A | 11:8A | 54:8A | 36:9A |
| 30:7A | 12:8A | 55:8A | 37:9A |
| 31:7A | 13:8A | 56:8A | 38:9A |
| 32:7A | 14:8A | 57:8A | 39:9A |
| 33:7A | 15:8A | 58:8A | 40:9A |
| 34:7A | 16:8A | 59:8A | 41:9A |
| 35:7A | 17:8A | 60:8A | 42:9A |
| 36:7A | 18:8A | 61:8A | 43:9A |
| 37:7A | 14:8A | 62:8A | 44:9A |
| 38:7A | 19:8A | 1:9A | 45:9A |
| 39:7A | 20:8A | 2:9A | 46:9A |
| 40:7A | 21:8A | 3:9A | 47:9A |
| 41:7A | 22:8A | 4:9A | 48:9A |
| 42:7A | 23:8A | 5:9A | 49:9A |
| 43:7A | 24:8A | 6:9A | 50:9A |
| 44:7A | 25:8A | 7:9A | 51:9A |
| 45:7A | 26:8A | 8:9A | 52:9A |
| 46:7A | 27:8A | 9:9A | 53:9A |
| 47:7A | 28:8A | 10:9A | 54:9A |
| 48:7A | 29:8A | 11:9A | 55:9A |
| 49:7A | 30:8A | 12:9A | 56:9A |
| 50:7A | 31:8A | 13:9A | 57:9A |
| 51:7A | 32:8A | 14:9A | 58:9A |
| 52:7A | 33:8A | 15:9A | 59:9A |
| 53:7A | 34:8A | 16:9A | 60:9A |
| 54:7A | 35:8A | 17:9A | 61:9A |
| 62:9A | 44:10A | 26:11A | 8:12A |
| 1:10A | 45:10A | 27:11A | 9:12A |
| 2:10A | 46:10A | 28:11A | 10:12A |
| 3:10A | 47:10A | 29:11A | 11:12A |
| 4:10A | 48:10A | 30:11A | 12:12A |
| 5:10A | 49:10A | 31:11A | 13:12A |
| 6:10A | 50:10A | 32:11A | 14:12A |
| 7:10A | 51:10A | 33:11A | 15:12A |
| 8:10A | 52:10A | 34:11A | 16:12A |
| 9:10A | 53:10A | 35:11A | 17:12A |
| 10:10A | 54:10A | 36:11A | 18:12A |
| 11:10A | 55:10A | 37:11A | 19:12A |
| 12:10A | 56:10A | 38:11A | 20:12A |
| 13:10A | 57:10A | 39:11A | 21:12A |
| 14:10A | 58:10A | 40:11A | 22:12A |
| 15:10A | 59:10A | 41:11A | 23:12A |
| 16:10A | 60:10A | 42:11A | 24:12A |
| 17:10A | 61:10A | 43:11A | 25:12A |
| 18:10A | 62:10A | 44:11A | 26:12A |
| 19:10A | 1:11A | 45:11A | 27:12A |
| 20:10A | 2:11A | 46:11A | 28:12A |
| 21:10A | 3:11A | 47:11A | 29:12A |
| 22:10A | 4:11A | 48:11A | 30:12A |
| 23:10A | 5:11A | 49:11A | 31:12A |
| 24:10A | 6:11A | 50:11A | 32:12A |
| 25:10A | 7:11A | 51:11A | 33:12A |
| 26:10A | 8:11A | 52:11A | 34:12A |
| 27:10A | 9:11A | 53:11A | 35:12A |
| 28:10A | 10:11A | 54:11A | 36:12A |
| 29:10A | 11:11A | 55:11A | 37:12A |
| 30:10A | 12:11A | 56:11A | 38:12A |
| 31:10A | 13:11A | 57:11A | 39:12A |
| 32:10A | 14:11A | 58:11A | 40:12A |
| 33:10A | 15:11A | 59:11A | 41:12A |
| 34:10A | 16:11A | 60:11A | 42:12A |
| 35:10A | 17:11A | 61:11A | 43:12A |
| 36:10A | 18:11A | 62:11A | 44:12A |
| 37:10A | 19:11A | 1:12A | 45:12A |
| 38:10A | 20:11A | 2:12A | 46:12A |
| 39:10A | 21:11A | 3:12A | 47:12A |
| 40:10A | 22:11A | 4:12A | 48:12A |
| 41:10A | 23:11A | 5:12A | 49:12A |
| 42:10A | 24:11A | 6:12A | 50:12A |
| 43:10A | 25:11A | 7:12A | 51:12A |
| 52:12A | 55:12A | 58:12A | 61:12A |
| 53:12A | 56:12A | 59:12A | 62:12A |
| 54:12A | 57:12A | 60:12A | |

The order of administration of compounds in a combination described herein can vary. In some embodiments, Compound (A), including pharmaceutically acceptable salts thereof, can be administered prior to all of Compound (B), or a pharmaceutically acceptable salt thereof. In other embodiments, Compound (A), including pharmaceutically acceptable salts thereof, can be administered prior to at least one Compound (B), or a pharmaceutically acceptable salt thereof. In still other embodiments, Compound (A), including pharmaceutically acceptable salts thereof, can be administered concomitantly with Compound (B), or a pharmaceutically acceptable salt thereof. In yet still other embodiments, Compound (A), including pharmaceutically acceptable salts thereof, can be administered subsequent to the administration of at least one Compound (B), or a pharmaceutically acceptable salt thereof. In some embodiments, Compound (A), including pharmaceutically acceptable salts thereof, can be administered subsequent to the administration of all Compound (B), or a pharmaceutically acceptable salt thereof.

There may be several advantages for using a combination of compounds described herein. For example, combining compounds that attack multiple pathways at the same time, can be more effective in treating a cancer, such as those described herein, compared to when the compounds of combination are used as monotherapy.

In some embodiments, a combination as described herein of Compound (A), including pharmaceutically acceptable salts thereof, and one or more of Compound (B), or pharmaceutically acceptable salts thereof, can decrease the number and/or severity of side effects that can be attributed to a compound described herein, such as Compound (B), or a pharmaceutically acceptable salt thereof.

Using a combination of compounds described herein can results in additive, synergistic or strongly synergistic effect. A combination of compounds described herein can result in an effect that is not antagonistic.

In some embodiments, a combination as described herein of Compound (A), including pharmaceutically acceptable salts thereof, and one or more of Compound (B), or pharmaceutically acceptable salts thereof, can result in an additive effect. In some embodiments, a combination as described herein of Compound (A), including pharmaceutically acceptable salts thereof, and one or more of Compound (B), or pharmaceutically acceptable salts thereof, can result in a synergistic effect. In some embodiments, a combination as described herein of Compound (A), including pharmaceutically acceptable salts thereof, and one or more of Compound (B), or pharmaceutically acceptable salts thereof, can result in a strongly synergistic effect. In some embodiments, a combination as described herein of Compound (A), including pharmaceutically acceptable salts thereof, and one or more of Compound (B), or pharmaceutically acceptable salts thereof, is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination as described herein may be a reduction in the required amount(s) of the compound(s) that is effective in treating a disease condition disclosed herein compared to when each compound is administered as a monotherapy. For example, the amount of Compound (B), or a pharmaceutically acceptable salt thereof, used in a combination described herein can be less compared to the amount of Compound (B), or a pharmaceutically acceptable salt thereof, needed to achieve the same reduction in a disease marker (for example, tumor size) when administered as a monotherapy. Another potential advantage of utilizing a combination as described herein is that the use of two or more compounds having different mechanisms of action can create a higher barrier to the development of resistance compared to when a compound is administered as monotherapy. Additional advantages of utilizing a combination as described herein may include little to no cross resistance between the compounds of a combination described herein; different routes for elimination of the compounds of a combination described herein; and/or little to no overlapping toxicities between the compounds of a combination described herein.

### Pharmaceutical Compositions

Compound (A), including pharmaceutically acceptable salts thereof, can be provided in a pharmaceutical composition. Likewise, Compound (B), including pharmaceutically acceptable salts thereof, can be provided in a pharmaceutical composition.

The term “pharmaceutical composition” refers to a mixture of one or more compounds and/or salts disclosed herein with other chemical components, such as diluents, carriers and/or excipients. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

In some embodiments, Compounds (B), along with pharmaceutically acceptable salts thereof, can be provided in a pharmaceutical composition that includes Compound (A), including pharmaceutically acceptable salts thereof. In other embodiments, Compound (B), along with pharmaceutically acceptable salts thereof, can be administered in a pharmaceutical composition that is separate from a pharmaceutical composition that includes Compound (A), including pharmaceutically acceptable salts thereof.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, Compound (A), including pharmaceutically acceptable salts thereof, can be administered orally. In some embodiments, Compound (A), including pharmaceutically acceptable salts thereof, can be provided to a subject by the same route of administration as Compound (B), along with pharmaceutically acceptable salts thereof. In other embodiments, Compound (A), including pharmaceutically acceptable salts thereof, can be provided to a subject by a different route of administration as Compound (B), along with pharmaceutically acceptable salts thereof.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

As provided herein, in some embodiments, a combination of compounds that includes an effective amount of Compound (A), including pharmaceutically acceptable salts thereof, and an effective amount of one or more of Compound (B), or a pharmaceutically acceptable salt of any of the foregoing, can be used to treat a disease or condition.

In some embodiments, the disease or condition can be selected from a brain cancer, a cervicocerebral cancer, an esophageal cancer, a thyroid cancer, a lung cancer, a breast cancer, a stomach cancer, a gallbladder/bile duct cancer, a liver cancer, a pancreatic cancer, a gastric cancer, a colon cancer, a rectal cancer, an ovarian cancer, an endometrial cancer, a choriocarcinoma, an uterus body cancer, an uterocervical cancer, a renal pelvis/ureter cancer, a bladder cancer, a prostate cancer, a penis cancer, a testicular cancer, a fetal cancer, an uterine cancer, Wilms’ cancer, a skin cancer, malignant melanoma, a neuroblastoma, an osteosarcoma, an Ewing’s tumor, a soft part sarcoma, a head and neck squamous cell carcinoma, a glioblastoma, an acute leukemia, a chronic lymphatic leukemia, a chronic myelocytic leukemia, polycythemia vera, a malignant lymphoma, multiple myeloma, a Hodgkin’s lymphoma and a non-Hodgkin’s lymphoma.

In some embodiments, the disease or condition can be a lung cancer (such as small cell lung cancer (SCLC) and/or non-small cell lung cancer (NSCLC)), a breast cancer (including triple negative breast cancer), a gastric cancer, a colon cancer, a rectal cancer, an ovarian cancer (for example, TP53-mutated ovarian cancer), an uterine cancer, an endometrial cancer, a head and neck squamous cell carcinoma and/or a glioblastoma. In some embodiments, the endometrial cancer can be an uterine serous carcinoma. In some embodiments, the disease or condition can be an osteosarcoma.

DNA damage repair (DDR) genes can play key roles in maintaining human genomic stability. Loss of DDR function, conversely, is an important determinant of cancer risk, progression and/or therapeutic response. DDR genes can be grouped into functional pathways defined by genetic, biochemical and mechanistic criteria. Proteins in the same pathway often work in concert to repair specific types of DNA damage. Base excision repair (BER), nucleotide excision repair (NER) and the direct damage reversal/repair (DR) pathways repair DNA base damage. Mismatch repair (MMR) can correct base mispairs and small loops that are often found in repetitive sequence DNA. Homology-dependent recombination (HR), non-homologous end joining (NHEJ), the Fanconi anemia (FA) pathway and translesion DNA synthesis (TLS) can act alone or together to repair DNA strand breaks and complex events, such as interstrand crosslinks. All of the major DDR pathways, with the exception of the FA pathway, have been identified in virtually all organisms. This reflects the universal need to counter the chemical instability of DNA and repair additional damage, such as those described herein.

Studies have shown that a homologous recombination deficiency (HRD) score can be predictive of defects in BRCA1 and/or BRCA2 gene. Several studies have been conducted to determine the potential correlation between a subject’s HRD score and the subject’s sensitivity to an anti-cancer agent. See Sharma et al., Annals of Oncology (2018) 29(3):645-660, Frey at al., Gynecologic Oncology Research and Practice (2017) 4:4, Hoppes et al., J Natl Cancer Inst (2018) 110(7):704-713 and Ledermann et al., Eur J Cancer (2016) 60:49-58. If a subject is determined to have HRD-positive status, the DNA of a subject may not be able to be repaired. In some embodiments, a subject utilizing a method and/or use described herein can have been determined to have homologous recombination deficiency (HRD) positive status. In other embodiments, a subject utilizing a method and/or use described herein can have been determined to have homologous recombination deficiency (HRD) negative status. In some embodiments, the subject has been diagnosed with a cancer selected from an ovarian cancer (including recurrent ovarian cancer), a breast cancer (such as triple-negative breast cancer and/or metastatic breast cancer), a prostate cancer (for example, a metastatic castration-resistant prostate cancer), a fallopian tube cancer and a primary peritoneal cancer. In some embodiments, the subject determined to have homologous recombination deficiency (HRD) positive status can be a woman. In some embodiments, the subject determined to have homologous recombination deficiency (HRD) positive status can be a man.

In some embodiments, a combination of Compound (1A) and a PARP inhibitor (including pharmaceutically acceptable salts of Compound (1A) and/or a PARP inhibitor) can be used to treat a subject that has homologous recombination deficiency (HRD) positive status. In other embodiments, a combination of Compound (1A) and a PARP inhibitor (including pharmaceutically acceptable salts of Compound (1A) and/or a PARP inhibitor) can be used to treat a subject that has a homologous recombination deficiency (HRD) negative status. In some embodiments, the combination of Compound (1A) and niraparib, along with pharmaceutically acceptable salts of any of the foregoing, can be used to treat a subject that has homologous recombination deficiency (HRD) positive status. In other embodiments, the combination of Compound (1A) and niraparib, along with pharmaceutically acceptable salts of any of the foregoing, can be used to treat a subject that has homologous recombination deficiency (HRD) negative status.

In some embodiments, a combination of Compound (1A) and niraparib (including pharmaceutically acceptable salts of Compound (1A) and/or a PARP inhibitor) can be used to treat an ovarian cancer in a subject that has homologous recombination deficiency (HRD) positive status. In other embodiments, a combination of Compound (1A) and niraparib (including pharmaceutically acceptable salts of Compound (1A) and/or a PARP inhibitor) can be used to treat an ovarian cancer in a subject that has homologous recombination deficiency (HRD) negative status. In some embodiments, a combination of Compound (1A) and niraparib, along with pharmaceutically acceptable salts of any of the foregoing, can be used to treat a breast cancer in a subject that has homologous recombination deficiency (HRD) positive status. In other embodiments, a combination of Compound (1A) and niraparib, along with pharmaceutically acceptable salts of any of the foregoing, can be used to treat a breast cancer in a subject that has homologous recombination deficiency (HRD) negative status. In some embodiments, a combination of Compound (1A) and niraparib (along with pharmaceutically acceptable salts of any of the foregoing) can be used to treat a prostate cancer in a subject that has homologous recombination deficiency (HRD) positive status. In other embodiments, a combination of Compound (1A) and niraparib (along with pharmaceutically acceptable salts of any of the foregoing) can be used to treat a prostate cancer in a subject that has homologous recombination deficiency (HRD) negative status. In some embodiments, a combination of Compound (1A) and niraparib, including pharmaceutically acceptable salts of any of the foregoing, can be used to treat metastatic breast cancer in a subject that has homologous recombination deficiency (HRD) positive status. In other embodiments, a combination of Compound (1A) and niraparib, including pharmaceutically acceptable salts of any of the foregoing, can be used to treat metastatic breast cancer in a subject that has homologous recombination deficiency (HRD) negative status. In some embodiments, a combination of Compound (1A) and niraparib (along with pharmaceutically acceptable salts of any of the foregoing) can be used to treat a cancer of a fallopian tube in a subject that has homologous recombination deficiency (HRD) positive status. In other embodiments, a combination of Compound (1A) and niraparib (along with pharmaceutically acceptable salts of any of the foregoing) can be used to treat a cancer of a fallopian tube in a subject that has homologous recombination deficiency (HRD) negative status. In some embodiments, a combination of Compound (1A), or a pharmaceutically acceptable salt thereof, and niraparib, or a pharmaceutically acceptable salt thereof, can be used to treat a primary peritoneal cancer in a subject that has homologous recombination deficiency (HRD) positive status. In other embodiments, a combination of Compound (1A), or a pharmaceutically acceptable salt thereof, and niraparib, or a pharmaceutically acceptable salt thereof, can be used to treat a primary peritoneal cancer in a subject that has homologous recombination deficiency (HRD) negative status. In some embodiments, a combination of Compound (1A) and niraparib, along with pharmaceutically acceptable salts of any of the foregoing, can be used to treat recurrent ovarian cancer in a subject that has homologous recombination deficiency (HRD) positive status. In other embodiments, a combination of Compound (1A) and niraparib, along with pharmaceutically acceptable salts of any of the foregoing, can be used to treat recurrent ovarian cancer in a subject that has homologous recombination deficiency (HRD) negative status. In some embodiments, a combination of Compound (1A) and niraparib (including pharmaceutically acceptable salts of any of the foregoing) can be used to treat metastatic castration-resistant prostate cancer in a subject that has homologous recombination deficiency (HRD) positive status. In other embodiments, a combination of Compound (1A) and niraparib (including pharmaceutically acceptable salts of any of the foregoing) can be used to treat metastatic castration-resistant prostate cancer in a subject that has homologous recombination deficiency (HRD) negative status.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject’s overall feeling of well-being or appearance.

The term “effective amount” is used to indicate an amount of an active compound, or pharmaceutical agent, that elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor.

The amount of compound, salt and/or composition required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compound of Formulae (A) and/or (B), or pharmaceutically acceptable salts of the foregoing, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### CTG Assay

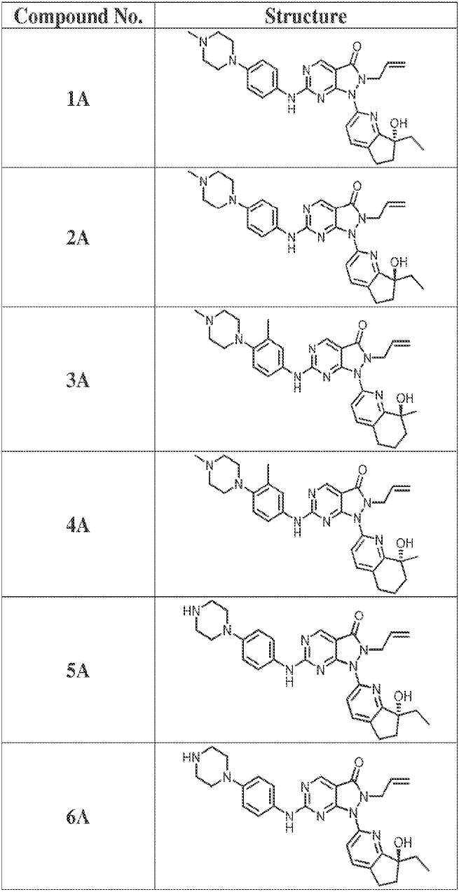

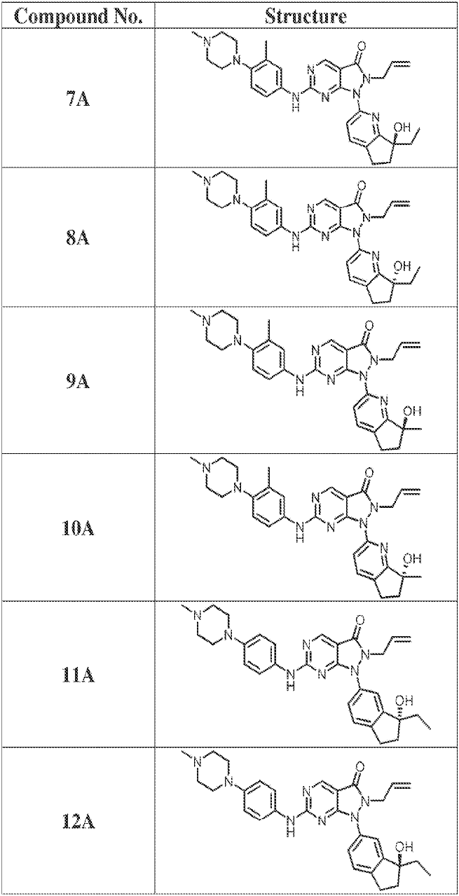

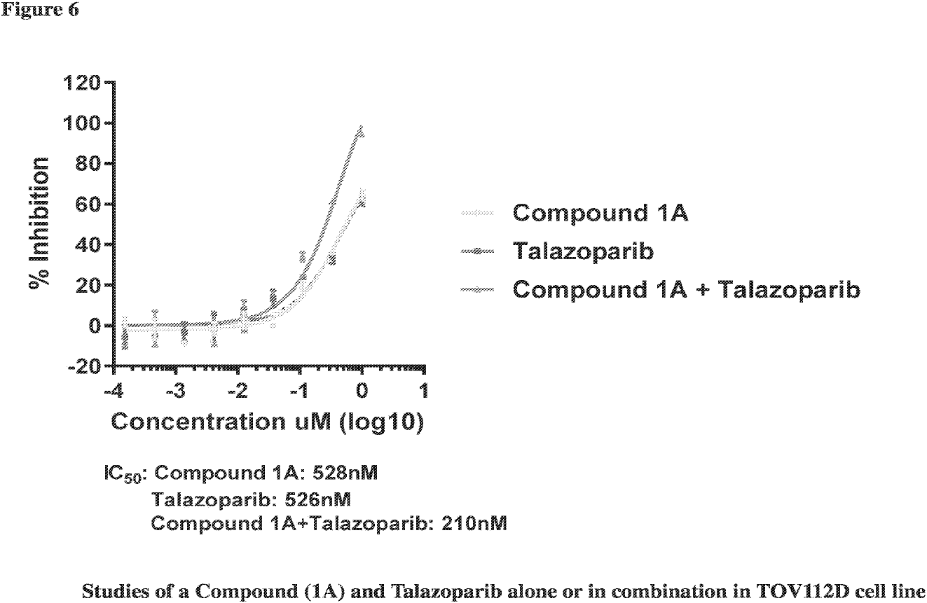

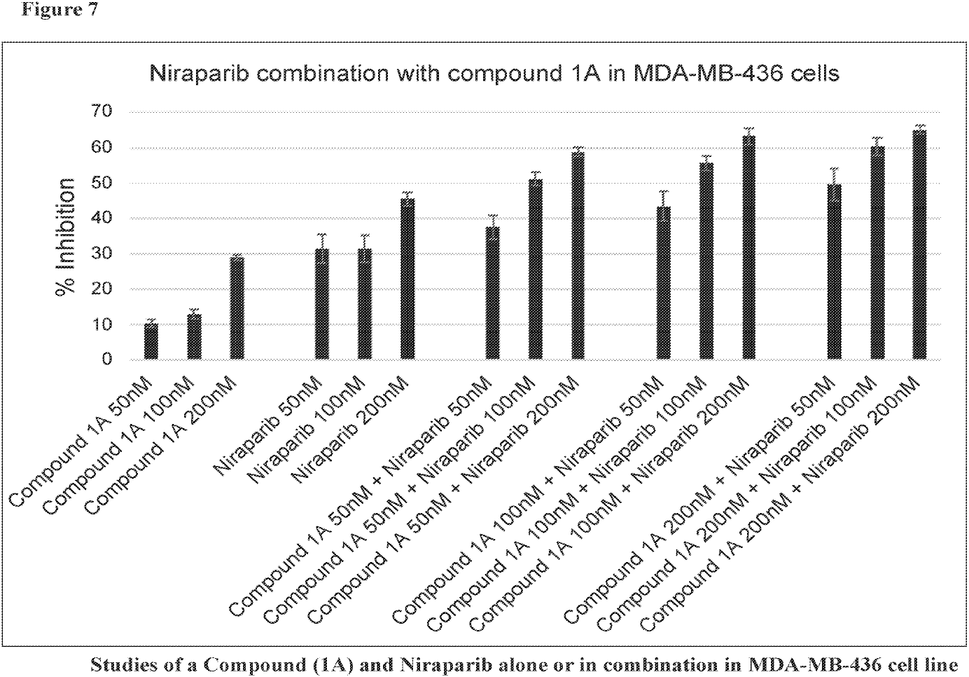

TOV112D cells were cultured in complete growth medium containing 15% fetal bovine serum and the base medium of 1:1 mixture of MCDB 105 medium with a final concentration of 1.5 g/L sodium bicarbonate and medium 199 with a final concentration of 2.2 g/L sodium bicarbonate. MDA-MB-436 cells were cultured in RPMI-1640 medium containing 10% fetal bovine serum. When cells were in exponential growth phase, cells were seeded in 96 cell plates and treated with indicated compounds at indicated concentrations of a single agent or in the combination. The anti-proliferative effect of test compounds was measured by CellTiter-Glo luminescence cell viability assay (Promega). Graphpad Prism software was used to generate IC₅₀ values. In FIG. 6, the upper-most line is Compound 1A + Talazoparib, the middle line indicated with circles is Compound 1A alone and bottom line indicated with squares is Talazoparib. The results for TOV112D and MDA-MB-436 cell lines are shown in FIG. 6 (Talazoparib - PARP inhibitor, TOV112D cell line), FIG. 7 (Niraparib - PARP inhibitor, MDA-MB-436 cell line). FIGS. 6-7 demonstrate that a combination of a compound (A), Compound 1A, with and without PARP inhibitors effectively inhibits cell proliferation. For the avoidance of any doubt, “Compound 1A” and “Compound (1A)” as used herein represent the same compound, and no discrepancy between the two is implied, nor should be inferred.

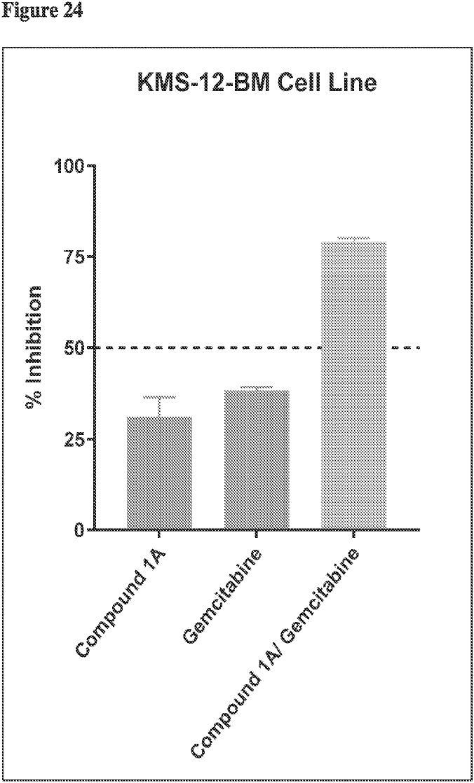

Cell proliferation was measured using the CellTiter-Glo® Luminescent Cell Viability Assay. The assay involved the addition of a single reagent (CellTiter-Glo® Reagent) directly to cells cultured in serum-supplemented medium. KMS-12-BM, OPM-2 and MOLP8 cells were cultured according to DSMZ recommendations and were seeded at 20,000 cells per well. Each compound evaluated was prepared as a DMSO stock solution (10 mM). Compounds were tested in triplicate on each plate, with a single concentration indicated in each table. Compound treatment (10.0 µL) was added to the cells from the 10x concentration of each compound. Plates were then incubated at 37° C., 5% CO₂. After 72 hrs, cell plates were equilibrated at room temperature (rt) for approximately 30 mins. An equi-volume amount of CellTiter-Glo® Reagent (100 µL) was added to each well. Plates were mixed for 2 mins on an orbital shaker to induce cell lysis and then incubated at rt for 10 mins to stabilize the luminescent signal. Luminescence was recorded using a SpectraMAX, M5e plate reader according to CellTiter-Glo protocol. Table 2 (and FIG. 24) illustrates that the combination of Compound (1A) and Gemcitabine in KMS-12-BM cell line showed synergistic cell proliferation inhibition compared to single agent treatment.

TABLE 2

KMS-12-BM

| | Concentration (nM) | Inhibition (%) |
|---|---|---|
| Compound (1A) | 300 | 31 |
| Gemcitabine | 90 | 38 |
| Compound (1A) + Gemcitabine | 300 + 90 | 79 |

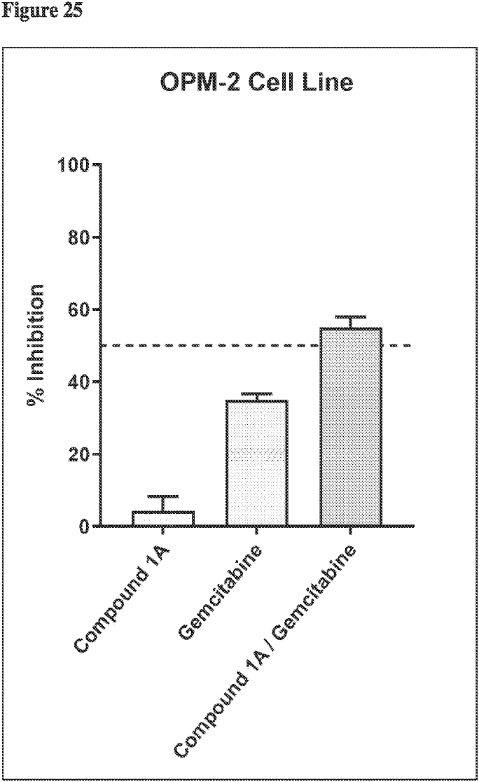

Table 3 (and FIG. 25) illustrates that the combination of Compound (1A) and Gemcitabine in OPM-2 cell line showed synergistic cell proliferation inhibition compared to single agent treatment.

TABLE 3

OPM-2

| | Concentration (nM) | Inhibition (%) |
|---|---|---|
| Compound (1A) | 300 | 4.3 |
| Gemcitabine | 50 | 35.1 |
| Compound (1A) + Gemcitabine | 300 + 50 | 55 |

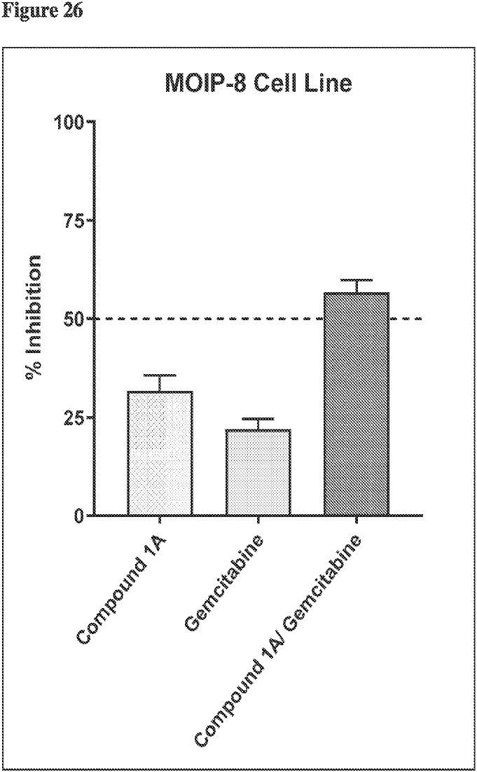

Table 4 (and FIG. 26) illustrates that the combination of Compound (1A) and Gemcitabine in MOLP-8 cell line showed synergistic cell proliferation inhibition compared to single agent treatment.

TABLE 4

MOLP-8

| | Concentration (nM) | Inhibition (%) |
|---|---|---|
| Compound (1A) | 150 | 32 |
| Gemcitabine | 2 | 22 |
| Compound (1A) + Gemcitabine | 150 + 2 | 57 |

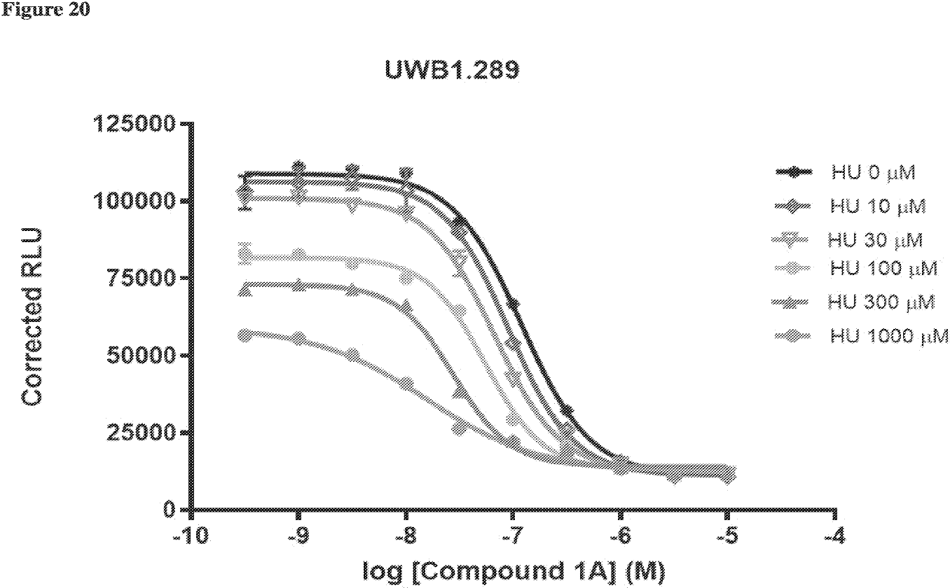

Ovarian cell lines (UWB 1.289 and OVCAR3) with moderate sensitivity to WEE1 inhibitor were treated with hydroxyurea (i.e. inducer of replication stress) and Compound (1A). 5,000 cells were plated per well in a 96-well plate. Compounds were prepared in DMSO (Compound (1A) at 10 µM starting concentration, with 1:3 dilution range. Hydroxyurea was added to cells at 0, 10, 30, 100, 300 or 1000 µM (matrix). Cells were then incubated at 37° C., 5% CO₂. CTG assay was performed after 3 days (for UWB1.289 cells) or 5 days (for OVCAR3 cells). Luciferase (relative light units, RLU) raw counts and normalized data are shown in FIGS. 20-23.

FIG. 20 shows inhibition of cell growth by Compound (1A) in combination with hydroxyurea (HU) against UWB 1.289 cells. Data is represented by relative light units (RLU). The data shows synergistic effects of hydroxyurea in combination with Compound (1A) in UWB1.289 cells. The HU 0 µm condition (top line with circles) indicates monotherapy with Compound (1A) as a reference. The HU 100 µm condition is the third from the bottom line with circles, and the HU 1000 µm condition is the bottom line with circles.

FIG. 21 shows inhibition of cell growth by Compound (1A) in combination with hydroxyurea (HU) against UWB 1.289 cells. Data is represented as relative light units (RLU) normalized for every hydroxyurea concentration, and shows synergistic effects for the combination with Compound (1A) and HU in UWB1.289 cells. The HU 0 µm condition (top line with circles) indicates monotherapy with Compound (1A) as a reference.

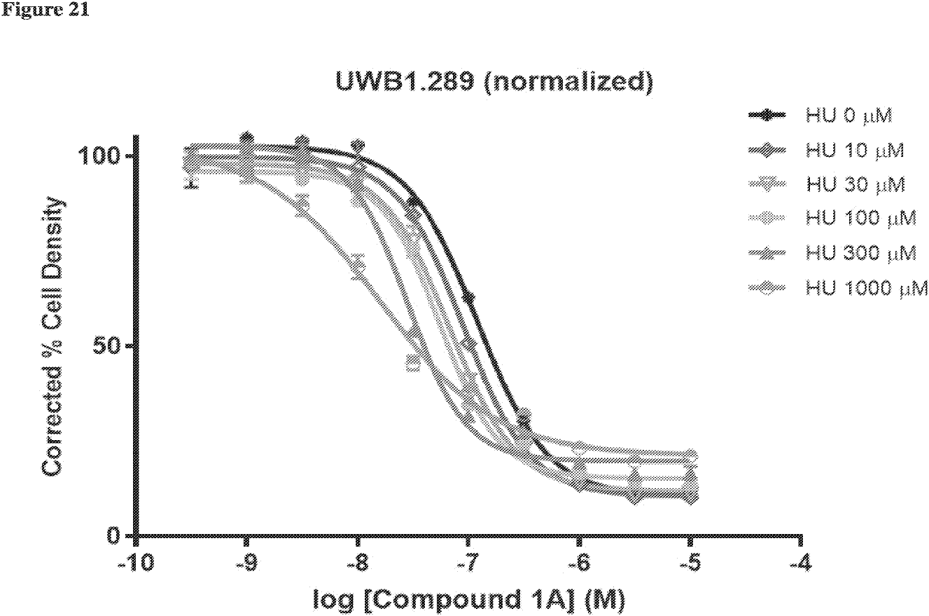

FIG. 22 shows inhibition of cell growth by Compound (1A) in combination with hydroxyurea (HU) against OVCAR3 cells. Data is represented by relative light units (RLU). The data shows synergistic effects of hydroxyurea in combination with Compound (1A) in OVCAR 3 cells. The HU 0 µm condition (top line with circles) indicates monotherapy with Compound (1A) as a reference. The HU 100 µm condition is the third from the bottom line with circles, and the HU 1000 µm condition is the bottom line with circles.

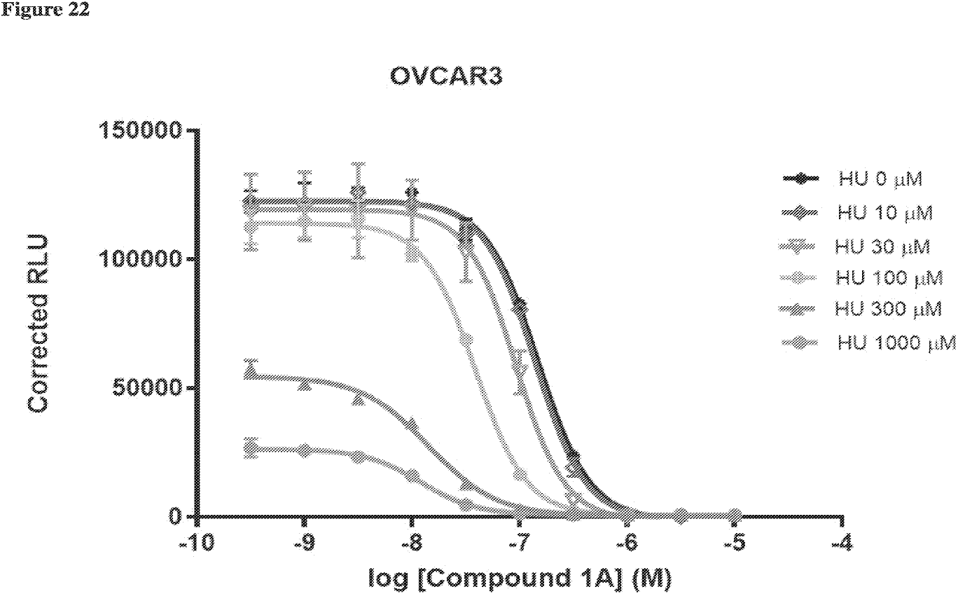

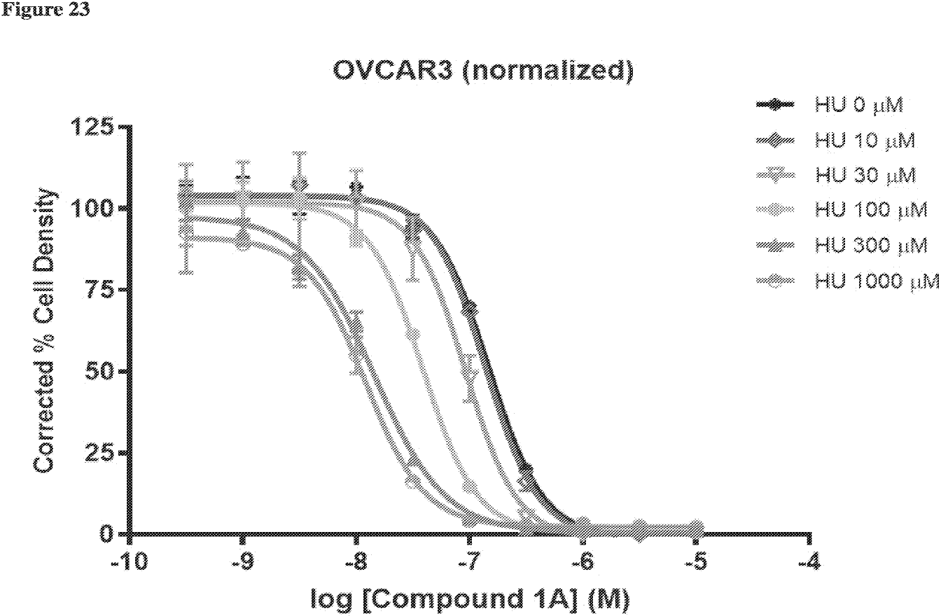

FIG. 23 shows inhibition of cell growth by Compound (1A) in combination with hydroxyurea (HU) against OVCAR3 cells. Data is represented as relative light units (RLU) normalized for every hydroxyurea concentration, and show synergistic effects for the combination with Compound (1A) and HU in OVCAR3 cells. The HU 0 µm condition (top line with circles) indicates monotherapy with Compound (1A) as a reference.

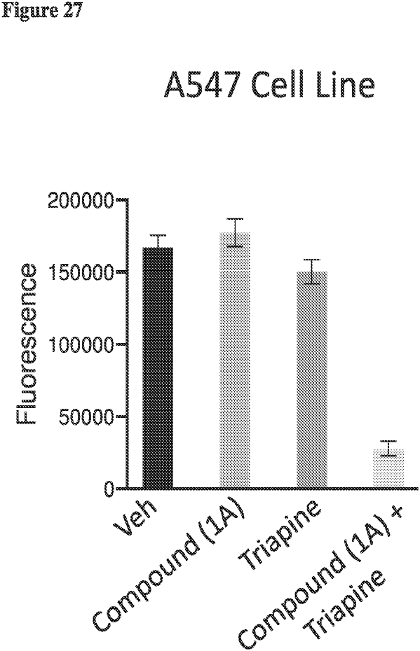

Experiment method: 3000 of A427 cells were seeded in 96-well pates and allowed to adhere overnight. Treatments with Compound (1A) and/or Triapine were added the next day. Cells were harvested at Day 6 and were assayed for DNA content using Hoechst 33258. Fluorescence intensity was read at excitation 346 nM and emission 460 nM using a plate reader. Data shown in FIG. 27 are representative of three independent experiments (raw fluorescence reading). As demonstrated by FIG. 27, suboptimal doses of Compound (1A) and Triapine as single agents do not inhibit A427 cell growth. In contrast, the combination of Compound (1A) and Triapine synergistically inhibits cell growth in A427 cells.

### Xenograft Tumor Model

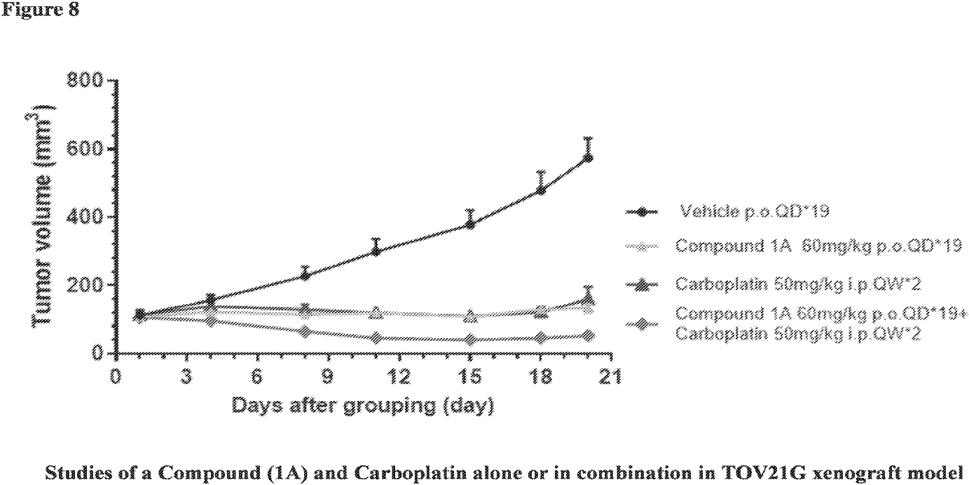

TOV21G xenograft model was established by the inoculation of 200 µL of TOV-21G tumor cell suspension (5×106 cells/mouse, with 50% Matrigel) subcutaneously into the right subaxillary of BALB/c nude mice. When tumors reached approximately 100 to 150 mm³, tumor bearing animals were randomly distributed into treatment groups of 10 animals each. Animals were orally dosed with vehicle or Compound (1A) at 60 mg/kg for 19 days, carboplatin at 50 mg/kg by the i.p. injection once per week, and Compound (1A) treatment in combination with carboplatin. Tumor volumes were evaluated twice per week to calculate tumor volume over time, and the mice were weighed twice per week as a surrogate for signs of toxicity. The results are provided in FIG. 8. As shown in FIG. 8, the combination treatment of Compound (1A) with carboplatin induced significant tumor regression with TGI value 117%, Compound (1A) and carboplatin as single agents result anti-tumor activities with TGI values 94.4% and 89.75% respectively.

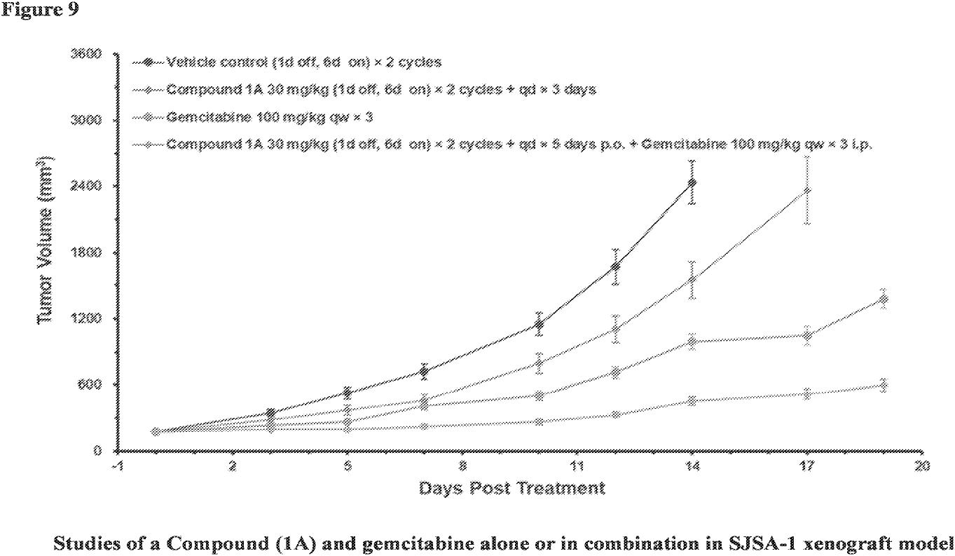

In SJSA-1 sarcoma subcutaneous xenograft efficacy study, mice were inoculated subcutaneously on right flank with SJSA-1 tumor cells. When mean tumor size reached approximately 150-200 mm³, animals were randomly distributed into treatment groups of 10 animals each and dosed with vehicle and indicated compounds at indicated dosage and frequency. Tumor volumes were evaluated twice per week to calculate tumor volume over time, and the mice were weighed twice per week as a surrogate for signs of toxicity. The results of the efficacy in SJSA-1 tumor model are shown in FIG. 9. In FIG. 9, the upper-most line is vehicle, the next upper-most line indicated with diamonds is Compound 1A alone, the next line indicated with circles is Gemcitabine alone and the bottom line is Compound 1A + Gemcitabine.

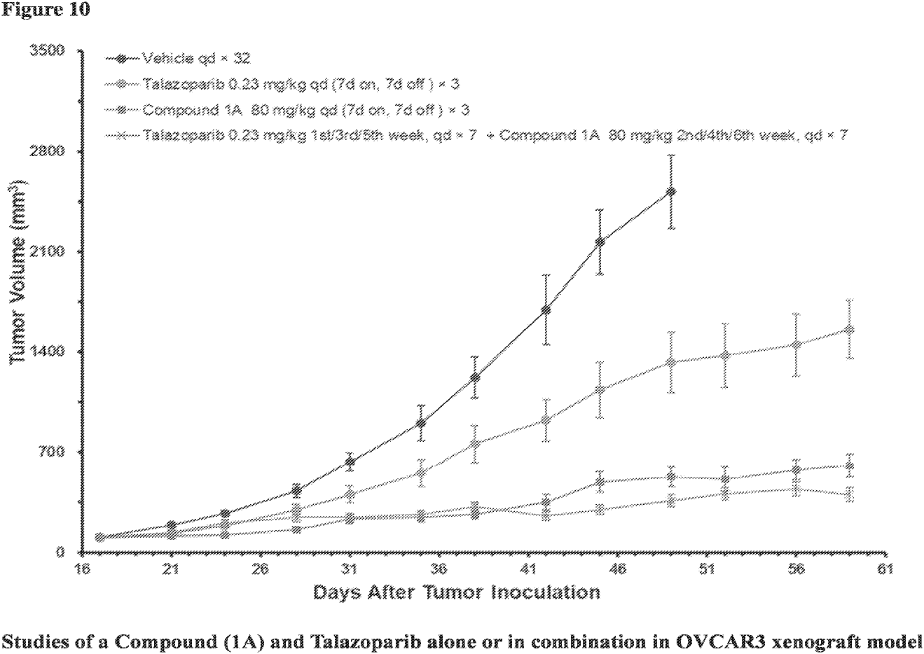

In OVCAR3 xenograft efficacy study, mice were implanted subcutaneously on right flank with OVCAR3 tumor cells. When tumors reached approximately 106 mm³, animals were randomly distributed into treatment groups of 10 animals each and dosed with vehicle and indicated compounds at indicated dosage and frequency. Tumor volumes were evaluated twice per week to calculate tumor volume over time, and the mice were weighed twice per week as a surrogate for signs of toxicity. The results of the OVCAR3 tumor model are shown in FIG. 10. In FIG. 10, the upper-most line is vehicle, the next upper-most line indicated with circles is Talazoparib alone, the next line indicated with squares is Compound 1A alone and the bottom line indicated with “x” is Compound 1A + Talazoparib.

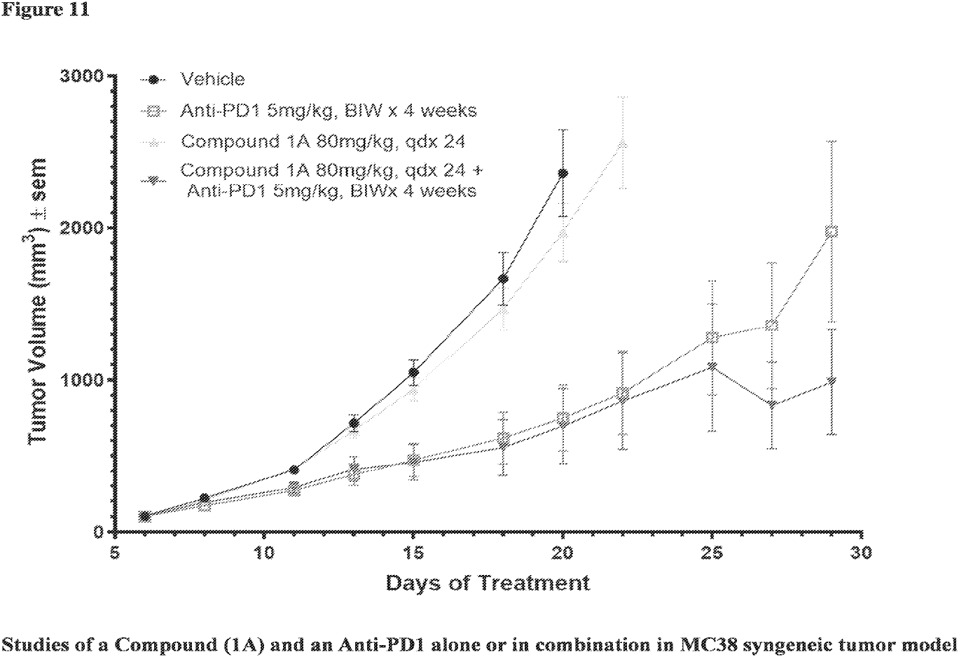

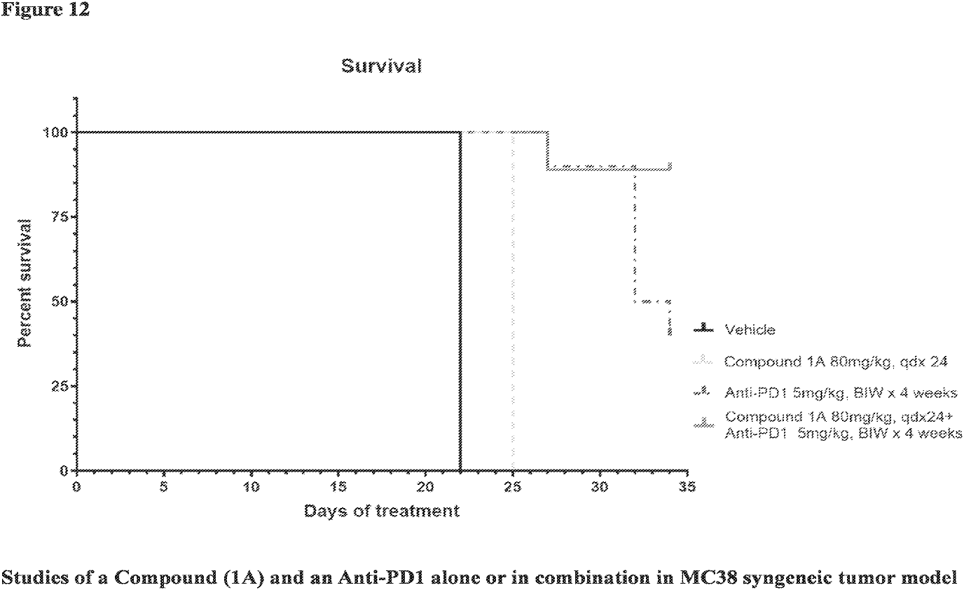

In a MC-38 syngeneic xenograft efficacy study, mice were implanted subcutaneously on the central right flank with MC38 tumor cells. When tumors reached approximately 102 mm³, animals were randomly distributed into treatment groups of 10 animals each and dosed with vehicle and indicated compounds or anti-PD-1 antibody (sourced from Pharmaron (BioXCell)) at indicated dosage and frequency. Tumor volumes were evaluated twice per week to calculate tumor volume over time, and the mice were weighed twice per week as a surrogate for signs of toxicity. The results of the MC38 syngeneic tumor model are shown in FIGS. 11 and 12. In FIG. 11, the upper-most line is vehicle, the next upper-most line indicated with triangles is Compound 1A alone, the next line indicated with open squares is an Anti-PD1 alone and the bottom line indicated with inverted triangles is Compound 1A + an Anti-PD1. In FIG. 12, the left-most solid line is vehicle, the next left-most line indicated with uniform dashes is Compound 1A, the next line solid line is an anti-PD1 alone and the next line indicated with alternating dots and dashes is Compound 1A + an Anti-PD1.

As shown in FIGS. 8-11, the combinations of a compound (A), Compound 1A, with PARP inhibitor (Talazoparib), or a chemotherapeutic agent, including carboplatin and gemcitabine, or an anti-PD1 antibody are effective in reducing tumor size. Furthermore, a combination of a compound (A), Compound 1A, and anti-PD1 antibody exhibits superior survival benefit than a single agent alone as shown in FIG. 12. A compound (A), Compound 1A, also is effective as a mono-therapeutic agent. For example, FIG. 8 demonstrates that a compound (A), Compound 1A, greatly reduces tumor volume.

The A-427 tumor cell line were maintained in vitro as monolayer culture in MEM Medium supplemented with 10% fetal bovine serum, 400 ng/mL puromycin at 37° C. in an atmosphere of 5% CO₂ in air. The cells growing in an exponential growth phase were harvested and counted for tumor inoculation. Each NOD/SCID mouse was inoculated subcutaneously on the right flank with the single cell suspension of 95% viable tumor cells (1 × 10⁷) in 100 µL MEM Matrigel mixture (1:1 ratio) without serum for the tumor development. The treatment was started when mean tumor size reaches approximately 224 mm³. Mice were then randomized into groups and orally dosed with vehicle or Compound (1A) at 80 mg/kg for 28 days. Tumor volumes were evaluated twice per week to calculate tumor volume over time, and the mice were weighed twice per week as a surrogate for signs of toxicity. The results are shown in FIG. 13, where the vehicle is the top line and the bottom line is Compound (1A) at 80 mg/kg. As shown by the data in FIG. 13, Compound (1A) treatment achieved significant anti-tumor activity with a TGI value of 132.7%.

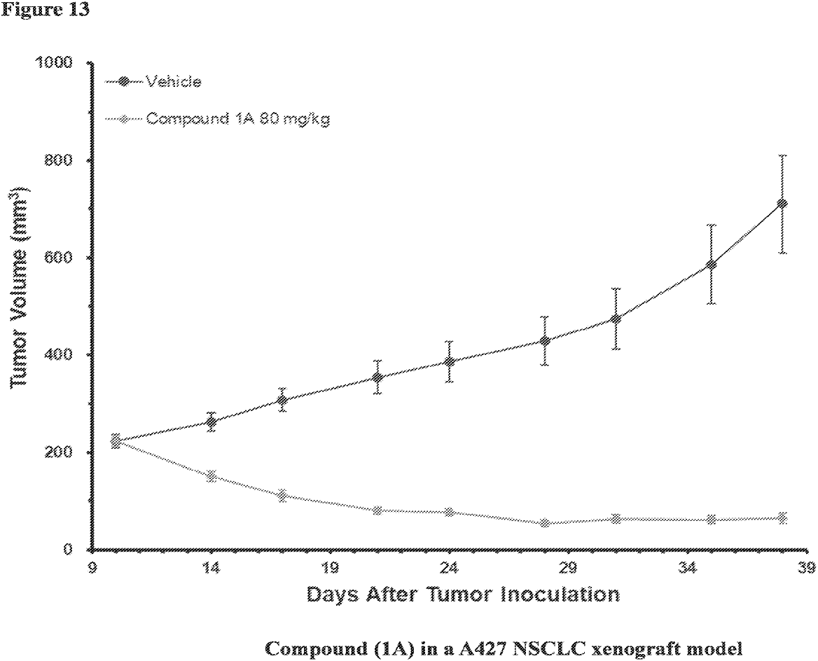

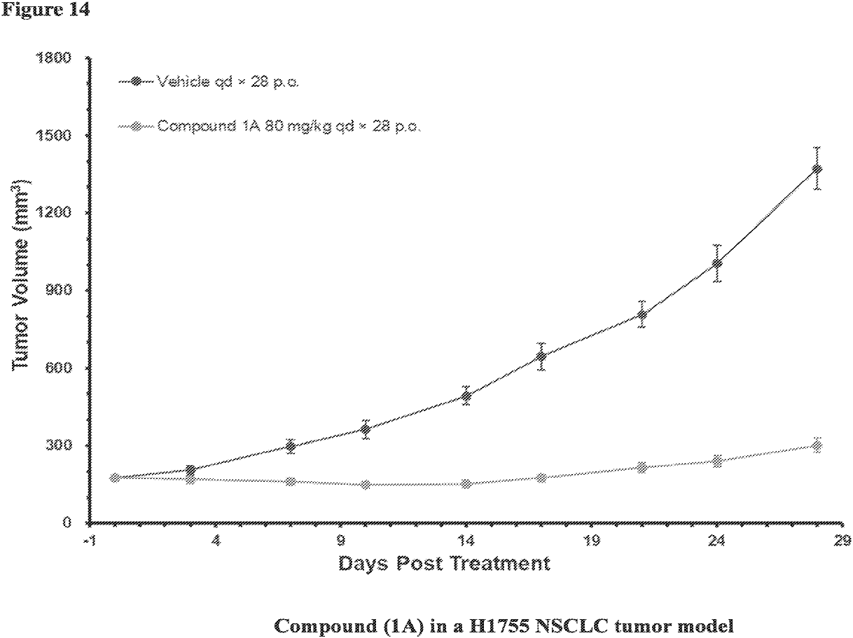

NCI-H1755 NSCLC cells were cultured in RPMI1640 medium supplemented with 10% fetal bovine serum at 37° C. in an atmosphere of 5% CO₂ in air. The cells growing in an exponential growth phase were harvested and counted for tumor inoculation. Each NOD SCID mouse was inoculated subcutaneously on the right flank with the single cell suspension of 95% viable tumor cells (1 × 107) in 100 µL RPMI1640 Matrigel mixture (1:1 ratio) without serum for the tumor development. The treatments were started when mean tumor size reached 176 mm³. Mice were randomized into treatment groups (10 mice per group). Vehicle or Compound (1A) at 80 mg/kg was orally administrated to the tumor-bearing mice for 28 days. Tumor volumes were evaluated twice per week to calculate tumor volume over time, and the mice were weighed twice per week as a surrogate for signs of toxicity. The results are shown in FIG. 14, wherein the vehicle is the top line and the bottom line is Compound (1A) at 80 mg/kg. The results demonstrate that Compound (1A) treatment as a single agent achieved significant anti-tumor activity with a TGI value of 89.6%.

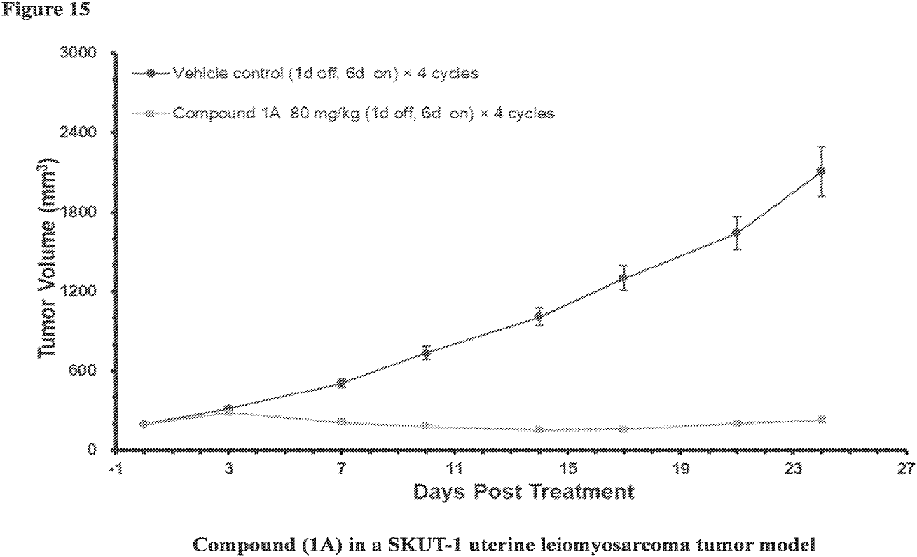

The SK-UT-1 tumor cell line was maintained in vitro as monolayer culture in EMEM supplemented with 10% fetal bovine serum at 37° C. in an atmosphere of 5% CO₂ in air. The cells growing in an exponential growth phase were harvested and counted for tumor inoculation. Each BALB/c nude mouse was inoculated subcutaneously on the right flank with the single cell suspension of 95% viable tumor cells (1 × 10⁷) in 100 µL EMEM Matrigel mixture (1:1 ratio) without serum for the tumor development. The treatments were started when mean tumor size reached 193 mm³. Mice were randomized into treatment groups (10 mice per group). Vehicle or Compound (1A) at 80 mg/kg was administrated to the tumor-bearing mice by 1 day on, 6 days off dosing schedule for 4 cycles. Tumor volumes were evaluated twice per week to calculate tumor volume over time, and the mice were weighed twice per week as a surrogate for signs of toxicity. The results are shown in FIG. 15, wherein the vehicle is the top line and the bottom line is Compound (1A) at 80 mg/kg. As shown in FIG. 15, Compound (1A) monotherapy achieved significant anti-tumor activity with a TGI value of 98%.

OVCAR-3 tumor cell line was maintained in vitro as monolayer culture in RPMI 1640 supplemented with 20% fetal bovine serum at 37° C. in an atmosphere of 5% CO₂ in air. The cells growing in an exponential growth phase were harvested and counted for tumor inoculation. Each NOD/SCID mouse was inoculated subcutaneously on the right flank with the single cell suspension of 95% viable tumor cells (2 × 10⁷) in 200 µL RPMI 1640 Matrigel mixture (1:1 ratio) without serum for the tumor development. The treatments were started on Day 15 when mean tumor size reached 111 mm³. Mice were randomized into treatment groups (10 mice per group) and orally dosed with vehicle and Compound (1A) at 80 mg/kg for 28 days. Tumor volumes were evaluated twice per week to calculate tumor volume over time, and the mice were weighed twice per week as a surrogate for signs of toxicity. The results are shown in FIG. 16, wherein the vehicle is the top line and the bottom line is Compound (1A) at 80 mg/kg. As shown in FIG. 16, Compound (1A) demonstrated robust anti-tumor activity with a TGI value of 91.3%.

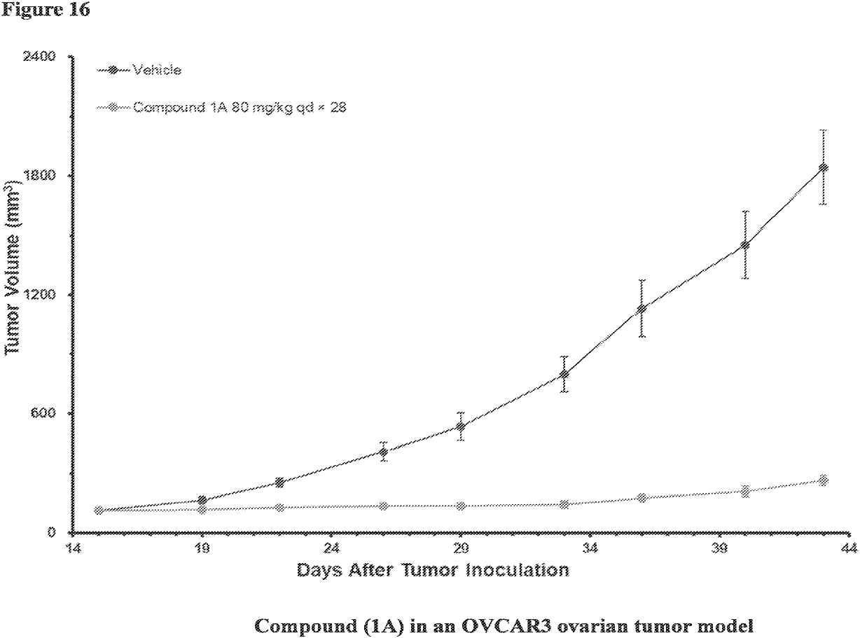

The x2-MDA-MB-468 cells (ATCC-Chempartner) were maintained in vitro as a monolayer culture in DMEM medium added with 10% FBS, 100U/mL penicillin and 100 µg/mL streptomycin at 37° C. in an atmosphere of 5% CO₂ in air. Each CB-17 SCID mouse was implanted subcutaneously on right flank with 1 × 10⁷ x2-MDA-MD-468 cells in 0.2 mL mixture of RPMI1640 medium with BD Matrigel (base medium: Matrigel=100 ul:100 ul) for tumor development. When the mean tumor size reached approximately 196 mm³, animals were randomly distributed into treatment groups of 10 animals each and orally dosed with vehicle, Compound (1A) at 80 mg/kg for 56 days. Tumor volumes were evaluated twice per week to calculate tumor volume over time, and the mice were weighed twice per week as a surrogate for signs of toxicity. The results are shown in FIG. 17. As shown in FIG. 17, Compound (1A) treatments resulted in significant antitumor activity with TGI a value of 87.1%.

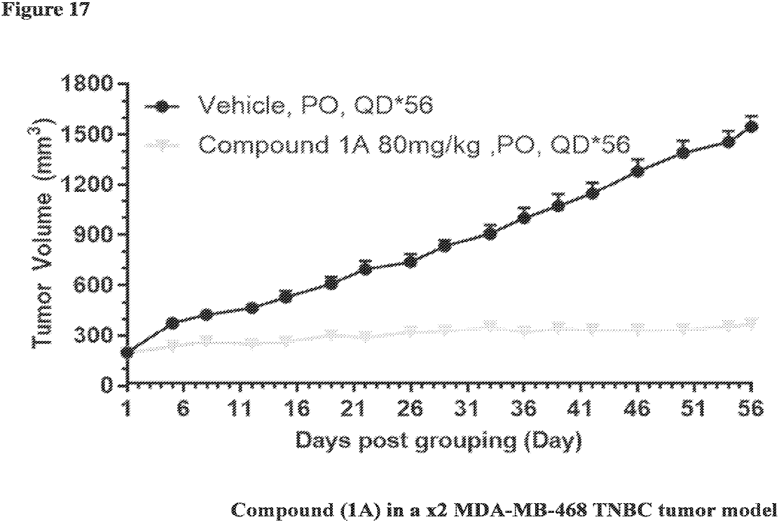

The x2-MDA-MB-468 cells (ATCC-Chempartner) were maintained in vitro as a monolayer culture in DMEM medium added with 10% FBS, 100 U/mL penicillin and 100 µg/mL streptomycin at 37° C. in an atmosphere of 5% CO₂ in air. Each mouse was implanted subcutaneously on right flank with 1 × 10⁷ x2-MDA-MD-468 cells in 0.2 mL mixture of RPMI1640 medium with BD Matrigel (base medium: Matrigel=100 uL:100 uL) for tumor development. When the mean tumor size reached approximately 196 mm³, animals were randomly distributed into treatment groups of 10 animals each and orally dosed with vehicle, Compound (1A) at 60 mg/kg, niraparib at 45 mg/kg, and Compound (1A) in combination with niraparib. Compound (1A) or niraparib was dosed at 7 days on, 7 days off regimen for 4 cycles as single agents. In the combination group, animals were dosed with niraparib and Compound (1A) in an alternative dosing schedule, niraparib was dosed at 1^{st}, 3^{rd}, 5^{th} and 7^{th} week, Compound (1A) was dosed at 2^{nd}, 4^{th}, 6^{th}, 8^{th} week. Tumor volumes were evaluated twice per week to calculate tumor volume over time, and the mice were weighed twice per week as a surrogate for signs of toxicity. The results are shown in FIG. 18. As shown in FIG. 18, the combination treatment of Compound (1A) with niraparib induced significant anti-tumor activity compared to Compound (1A) and niraparib as single treatments. Compound (1A) single treatment, niraparib single treatment and Compound (1A) in combination with niraparib produced antitumor activity with TGI values of 52.6%, 47.7% and 70.7%, respectively.

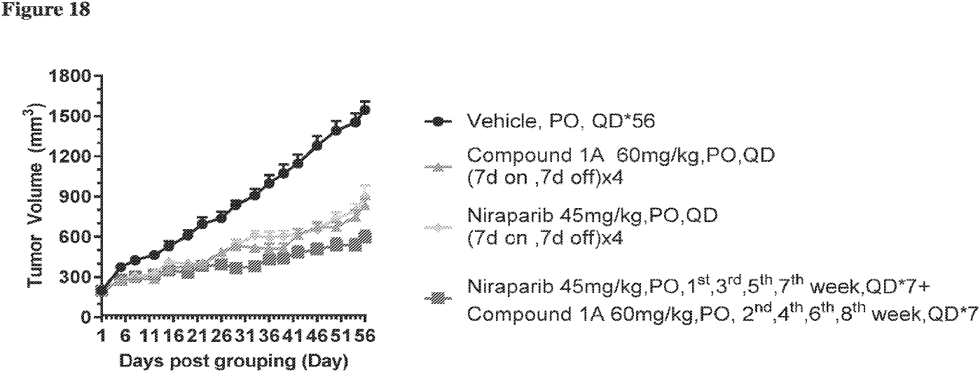

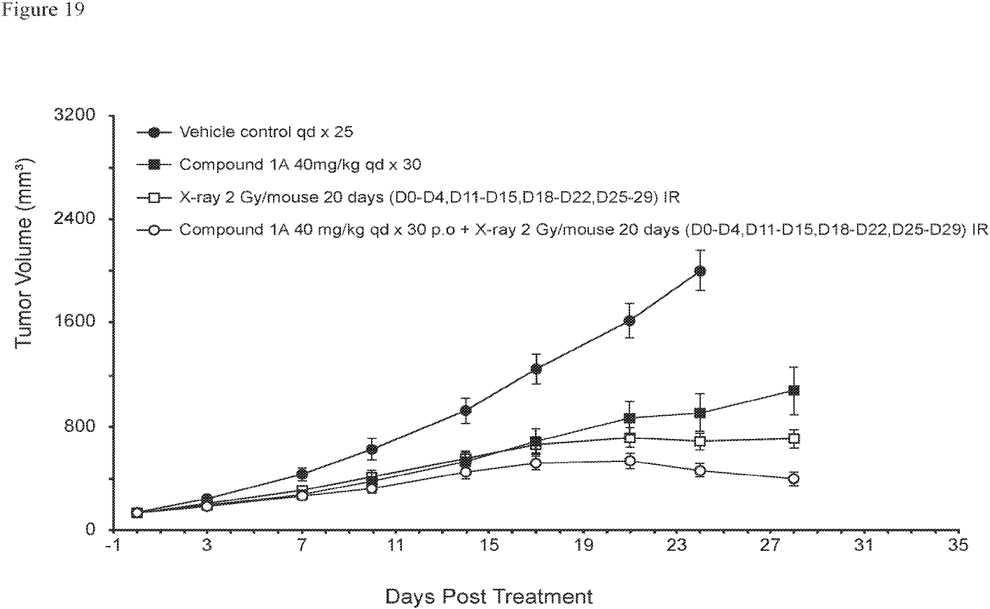

Fadu cells were grown in EMEM Medium supplemented with 20% fetal bovine serum at 37° C. in an atmosphere of 5% CO₂ in air. BALB/c nude mice were implanted subcutaneously on the right flank with a single cell suspension of 95% viable tumor cells (5 × 10⁶) in 100 µL EMEM with 10% FBS. When tumors reached approximately 138 mm³, animals were randomly distributed into treatment groups of 10 animals each and treated as follows: vehicle dosed for 25 days, Compound (1A) orally dosed for 30 days at 40 mg/kg once per day, X-ray treated at 2 Gy/mouse for 5 days on, 7 days off, then followed up with 5 days on, 2 days off fractioned irradiation schedule for 3 cycles, and Compound (1A) in combination with X-ray. Tumor volumes were evaluated twice per week to calculate tumor volume over time, and mice were weighed twice per week as a surrogate for signs of toxicity. The results are shown in FIG. 19. As shown in FIG. 19, the combination of Compound (1A) and X-ray is more effective in reducing tumor size compared to Compound (1A) and X-ray as single treatments. Compound (1A) single treatment, X-ray single treatment and Compound (1A) in combination with X-ray produced antitumor activity with TGI values of 58.7%, 70.7% and 82.6%, respectively.

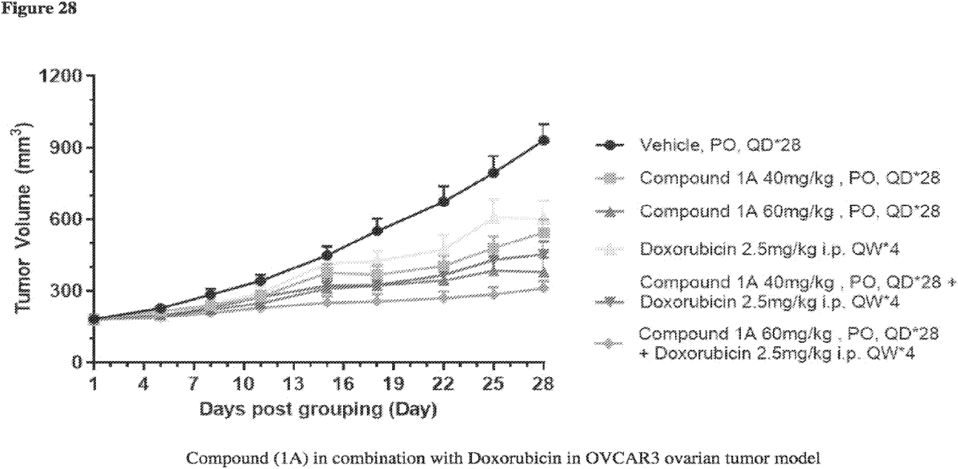

OVCAR3 xenograft model was established by the inoculation of 200 µL of OVCAR3 tumor cell suspension (1×10⁷ cells/mouse, with 50% Matrigel) subcutaneously into the right subaxillary of BABL/c nude mice. When tumors reached approximately 180.8 mm³, tumor bearing animals were randomly distributed into treatment groups of 10 animals each. Animals were orally dosed with vehicle or Compound (1A) at 40 mg/kg or 60 mg/kg for 28 days, doxorubicin at 2.5 mg/kg by the i.p. injection once per week for 4 weeks, and Compound (1A) treatment in combination with doxorubicin. Tumor volumes were evaluated twice per week to calculate tumor volume over time, and the mice were weighed twice per week as a surrogate for signs of toxicity. The results are shown in FIG. 28. The combination treatment of Compound (1A) at 40 mg/kg or 60 mg/kg with 2.5 mg/kg doxorubicin induced improved antitumor activity with TGI values of 63.47% and 82.57%. By comparison, Compound (1A) at 40 mg/kg or 60 mg/kg and doxorubicin at 2.5 mg/kg as single agents results in anti-tumor activities with TGI values of 51.66%, 73.48% and 43.11% respectively. As the active pharmaceutical ingredient in pegylated liposomal doxorubicin is doxorubicin, it is reasonable to assume that pegylated liposomal doxorubicin in combination with Compound (1A) will show similar results.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

What is claimed is:

1. Use of a combination of compounds for treating a disease or condition, wherein the combination includes an effective amount of Compound (A) and an effective amount of one or more of Compound (B), or a pharmaceutically acceptable salt of any of the foregoing, wherein: the Compound (A) has the structure: 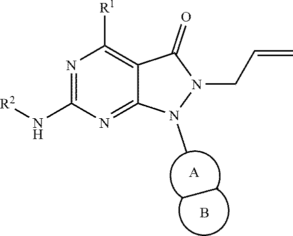
wherein:
R¹ is selected from the group consisting of hydrogen, halogen and a substituted or unsubstituted C₁-C₆ alkyl;
Ring A is selected from the group consisting of a substituted or unsubstituted phenyl and a substituted or unsubstituted 5-6 membered monocyclic heteroaryl;
Ring B is selected from the group consisting of a substituted or unsubstituted monocyclic 5-7 membered carbocyclyl and a substituted or unsubstituted 5-7 membered monocyclic heterocyclyl;
R² is selected from the group consisting of 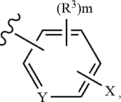 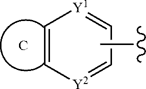
and 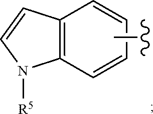
m is 0, 1, 2 or 3;
R³ is selected from the group consisting of halogen and a substituted or unsubstituted C₁-C₆ alkyl;
X is selected from the group consisting of hydrogen, halogen, hydroxy, cyano, a substituted or unsubstituted 4-6 membered monocyclic heterocyclyl, a substituted or unsubstituted amine(C₁-C₆ alkyl), a substituted or unsubstituted —NH—(CH₂)₁₋₆-amine, a mono-substituted amine, a di-substituted amine, an amino, a substituted or unsubstituted C₁-C₆ alkyl, a substituted or unsubstituted C₁-C₆ alkoxy, a substituted or unsubstituted C₃-C₆ cycloalkoxy, a substituted or unsubstituted (C₁-C₆ alkyl)acyl, a substituted or unsubstituted C-amido, a substituted or unsubstituted N-amido, a substituted or unsubstituted C-carboxy, a substituted or unsubstituted O-carboxy, a substituted or unsubstituted O-carbamyl and a substituted or unsubstituted N-carbamyl;
Y is CH or N;
Y¹ is CR^{4A} or N;
Y² is CR^{4B} or N;
Ring C is selected from the group consisting of a substituted or unsubstituted C₆-C_{lO} aryl, a substituted or unsubstituted monocyclic 5-10 membered heteroaryl, a substituted or unsubstituted monocyclic 5-7 membered carbocyclyl, a substituted or unsubstituted 5-7 membered monocyclic heterocyclyl and a substituted or unsubstituted 7-10 membered bicyclic heterocyclyl;
R^{4A} and R^{4B} are independently selected from the group consisting of hydrogen, halogen and an unsubstituted Cₗ₋₄ alkyl; and
R⁵ is a substituted or unsubstituted 5-7 membered monocyclic heterocyclyl; and
the one or more of Compound (B) is selected from the group consisting of a PARP inhibitor, a PD1 inhibitor, a PD-L1 inhibitor and a chemotherapeutic agent, or a pharmaceutically acceptable salt of any of the foregoing;
wherein the PARP inhibitor is selected from the group consisting of olaparib, niraparib, rucaparib, talazoparib, veliparib, pamiparib (BGB-290), iniparib (BSI 201), E7016 (Esai) and CEP-9722, and pharmaceutically acceptable salts of any of the foregoing;
wherein the PD1 inhibitor is selected from the group consisting of nivolumab, pembrolizumab, cemiplimab, spartalizumab, ABBV-181, lodapolimab, zimberelimab, toripalimab (Tuoyi), tislelizumab, camrelizumab, sintilimab (Tyvyt), GB226, AK105, HLX-10, AK103, BAT-1306, GSL-010, CS1003, LZM009 and SCT-I10A, and pharmaceutically acceptable salts of any of the foregoing;
wherein the PD-L1 inhibitor is selected from the group consisting of atezolizumab, avelumab, durvalumab, KN035, CS1001, SHR-1316, TQB2450, BGB-A333, KL-A167, KN046, MSB2311 and HLX-20, and pharmaceutically acceptable salts of any of the foregoing; and
wherein the chemotherapeutic agent is selected from the group consisting of carboplatin, cisplatin, paclitaxel, docetaxel, pegylated liposomal doxorubicin, doxorubicin, gemcitabine, cytarabine, fludarabine, fluorouracil (5-FU), irinotecan, topotecan, temozolomide, triapine, 5-azacytidine, capecitabine, AraC-FdUMP[10] (CF-10), cladribine, decitabine, hydroxyurea and oxaliplatin, and pharmaceutically acceptable salts of any of the foregoing.

2. Use of an effective amount of Compound (A), or a pharmaceutically acceptable salt thereof, in combination with radiation for treating a disease or condition, wherein: the Compound (A) has the structure: 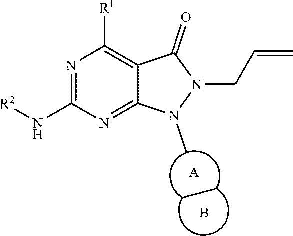
wherein:
R¹ is selected from the group consisting of hydrogen, halogen and a substituted or unsubstituted C₁-C₆ alkyl;
Ring A is selected from the group consisting of a substituted or unsubstituted phenyl and a substituted or unsubstituted 5-6 membered monocyclic heteroaryl;
Ring B is selected from the group consisting of a substituted or unsubstituted monocyclic 5-7 membered carbocyclyl and a substituted or unsubstituted 5-7 membered monocyclic heterocyclyl;
R² is selected from the group consisting of 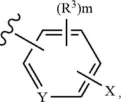 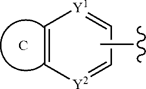
and 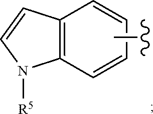
m is 0, 1, 2 or 3;
R³ is selected from the group consisting of halogen and a substituted or unsubstituted C₁-C₆ alkyl;
X is selected from the group consisting of hydrogen, halogen, hydroxy, cyano, a substituted or unsubstituted 4-6 membered monocyclic heterocyclyl, a substituted or unsubstituted amine(C₁-C₆ alkyl), a substituted or unsubstituted —NH—(CH₂)₁₋₆-amine, a mono-substituted amine, a di-substituted amine, an amino, a substituted or unsubstituted C₁-C₆ alkyl, a substituted or unsubstituted C₁-C₆ alkoxy, a substituted or unsubstituted C₃-C₆ cycloalkoxy, a substituted or unsubstituted (C₁-C₆ alkyl)acyl, a substituted or unsubstituted C-amido, a substituted or unsubstituted N-amido, a substituted or unsubstituted C-carboxy, a substituted or unsubstituted O-carboxy, a substituted or unsubstituted O-carbamyl and a substituted or unsubstituted N-carbamyl;
Y is CH or N;
Y¹ is CR^{4A} or N;
Y² is CR^{4B} or N;
Ring C is selected from the group consisting of a substituted or unsubstituted C₆-C_{lO} aryl, a substituted or unsubstituted monocyclic 5-10 membered heteroaryl, a substituted or unsubstituted monocyclic 5-7 membered carbocyclyl, a substituted or unsubstituted 5-7 membered monocyclic heterocyclyl and a substituted or unsubstituted 7-10 membered bicyclic heterocyclyl;
R^{4A} and R^{4B} are independently selected from the group consisting of hydrogen, halogen and an unsubstituted C₁₋₄ alkyl; and
R⁵ is a substituted or unsubstituted 5-7 membered monocyclic heterocyclyl.

3. The use of claim 1 or 2, wherein the Compound (A) is selected from the group consisting of: 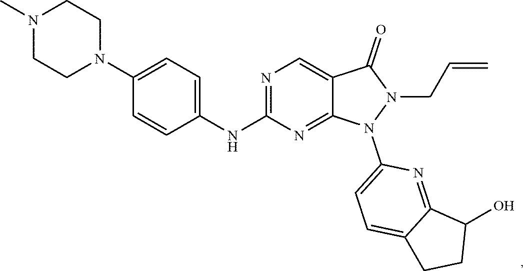 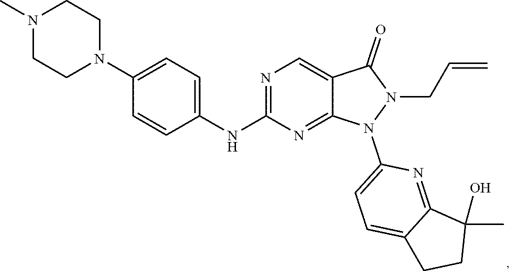 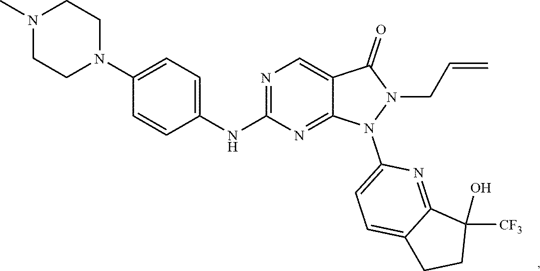 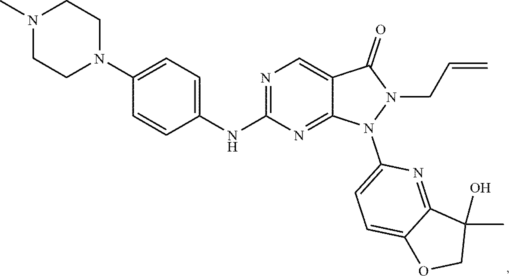 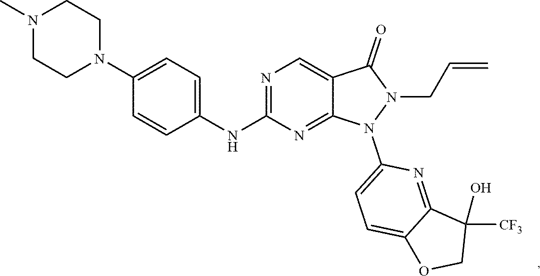 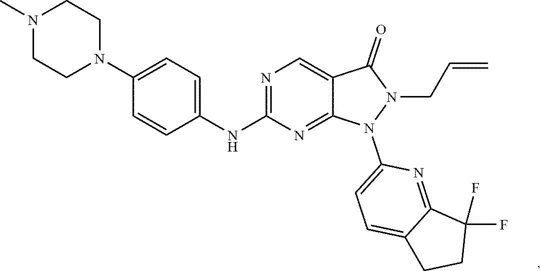 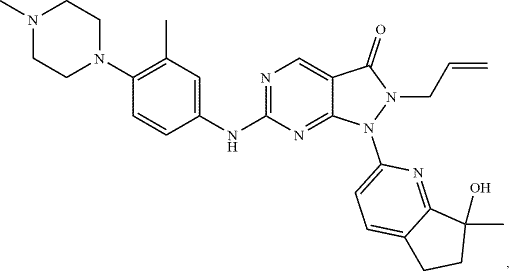 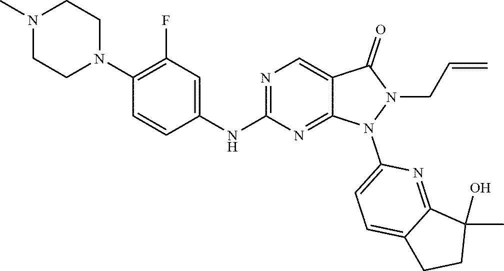
and 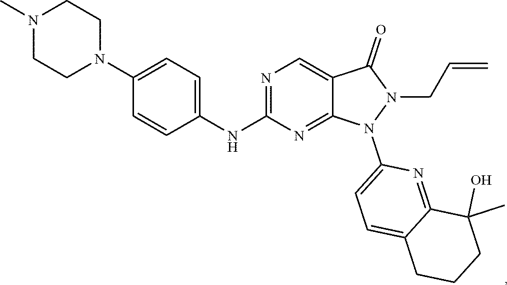
or a pharmaceutically acceptable salt of any of the foregoing.

4. The use of any one of claims 1-3, wherein the Compound (A) is selected from the group consisting of: 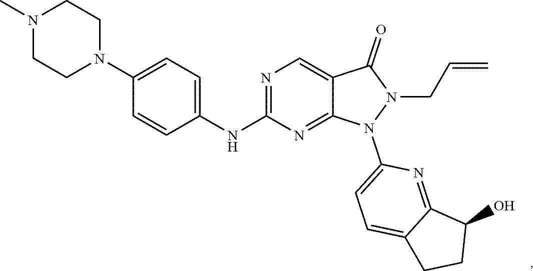 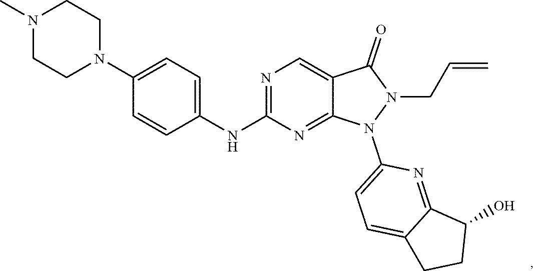 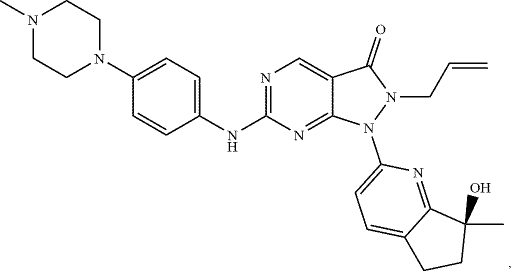 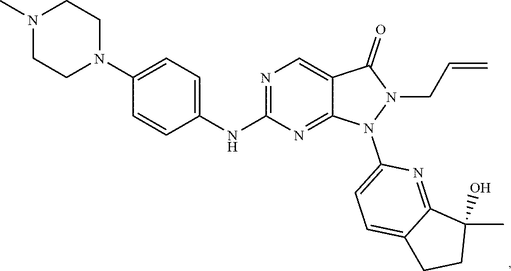 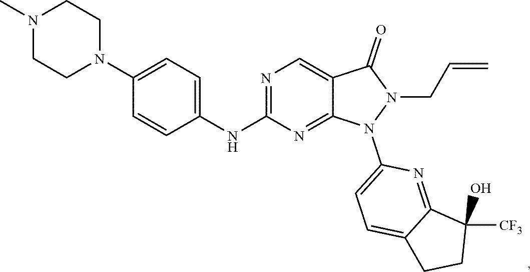 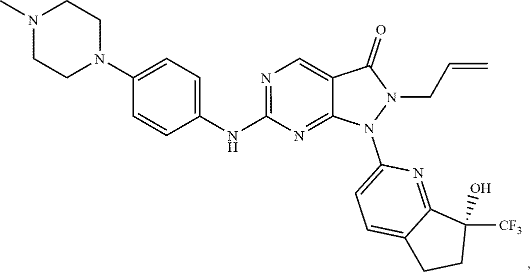 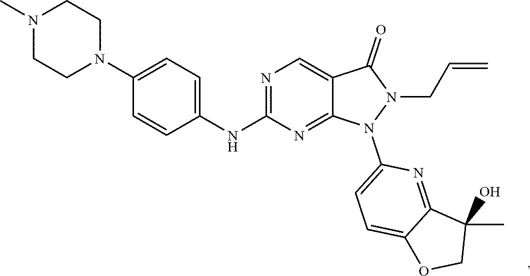 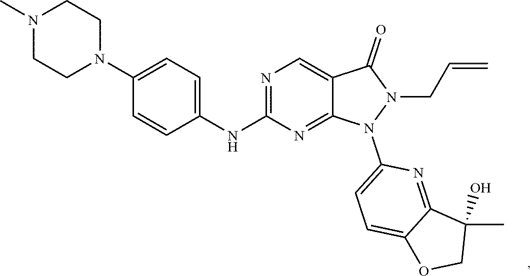 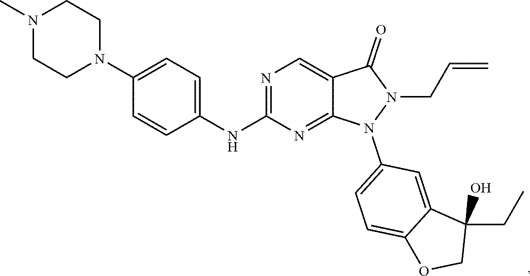 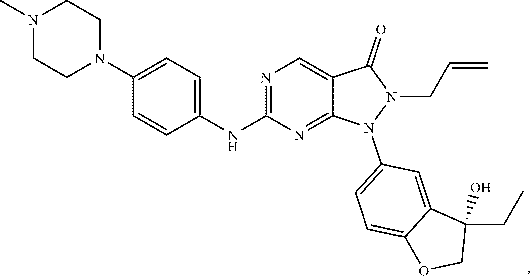 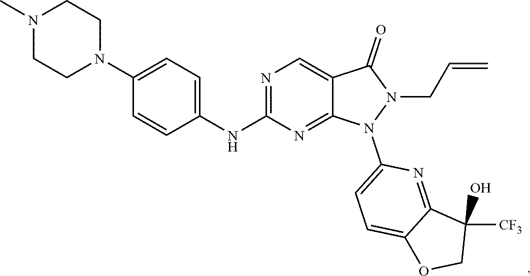 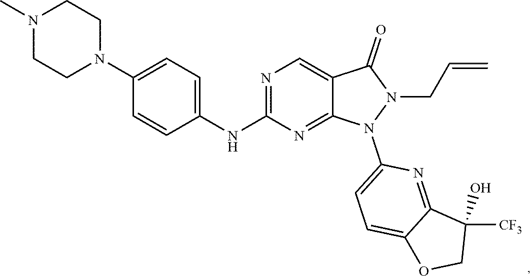 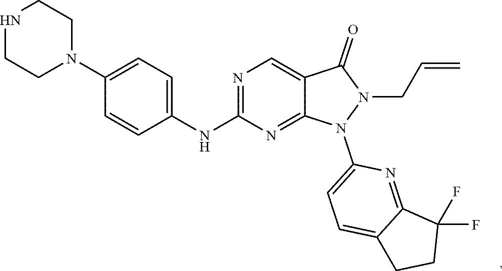 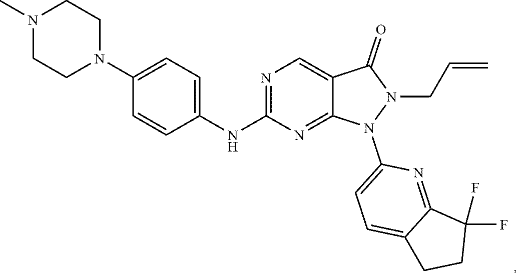 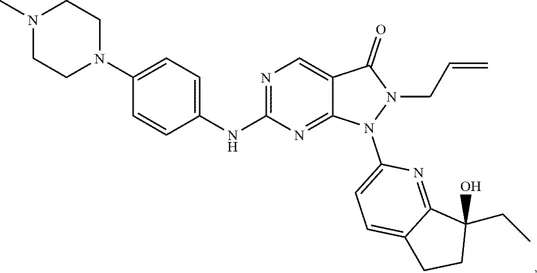 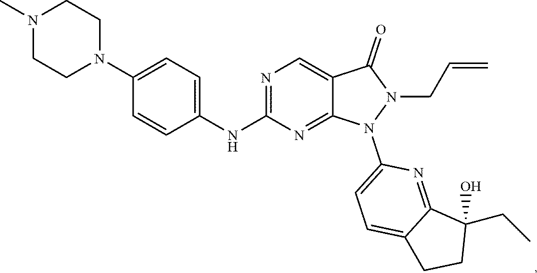 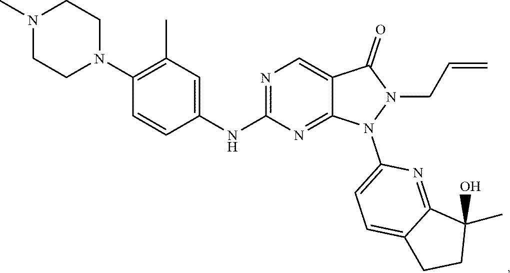 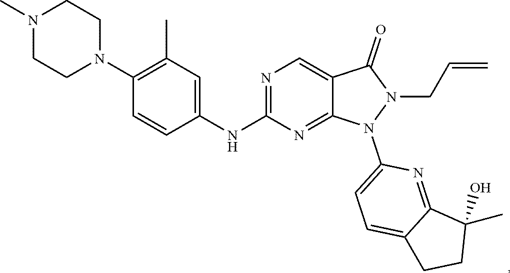 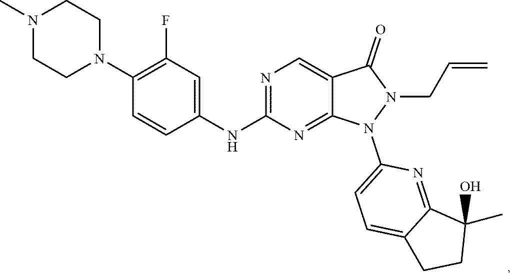 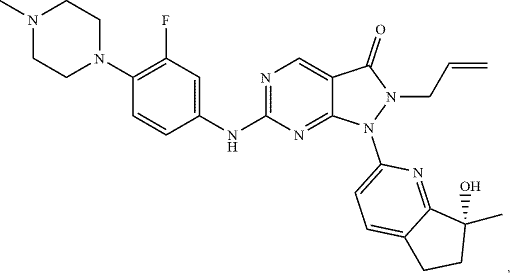 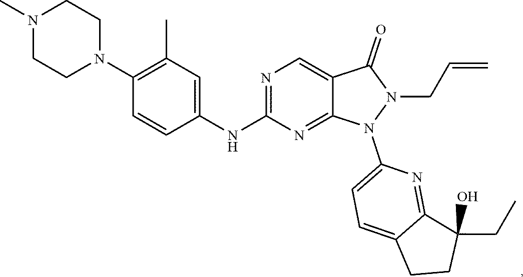 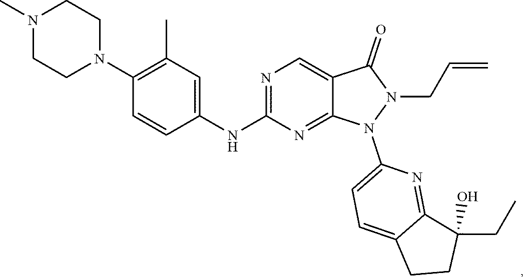 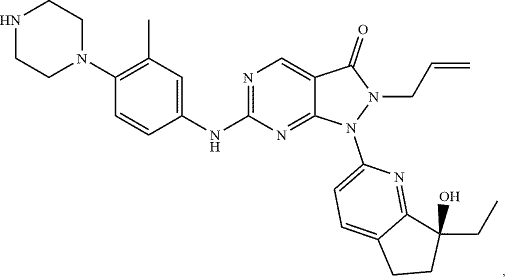 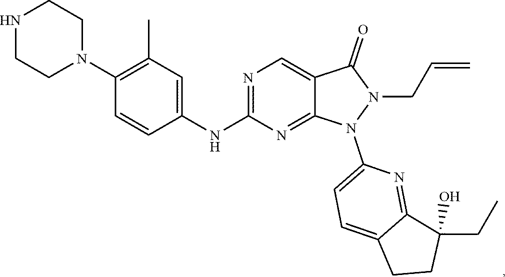 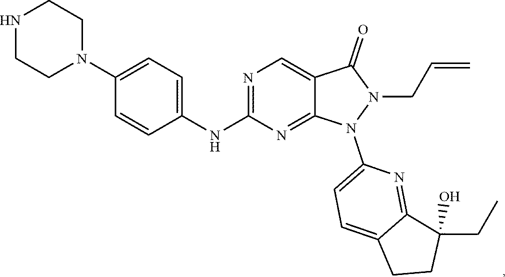 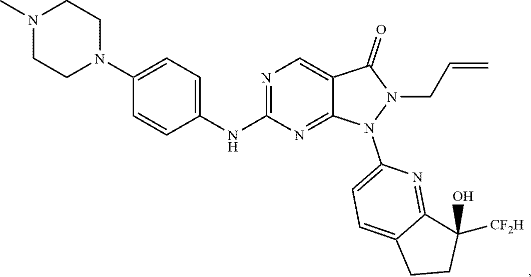 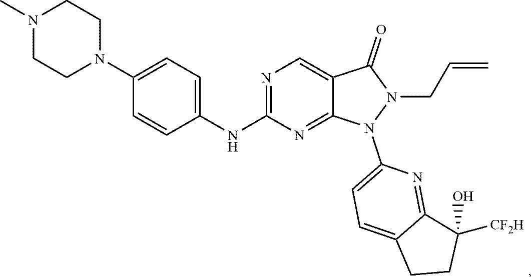 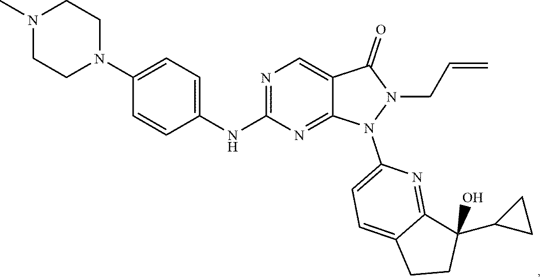 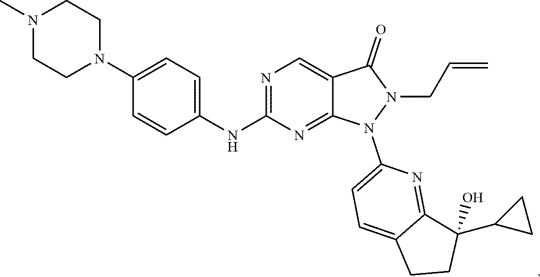 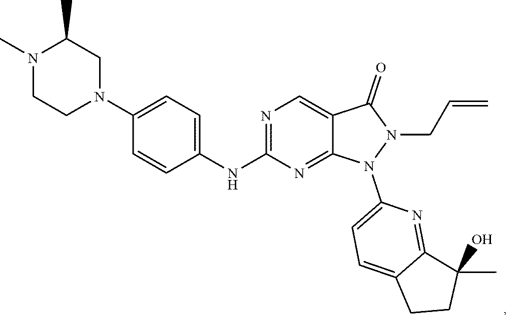 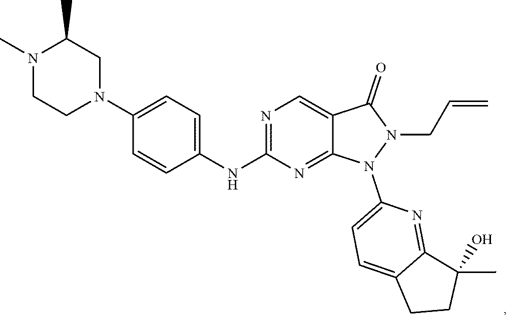 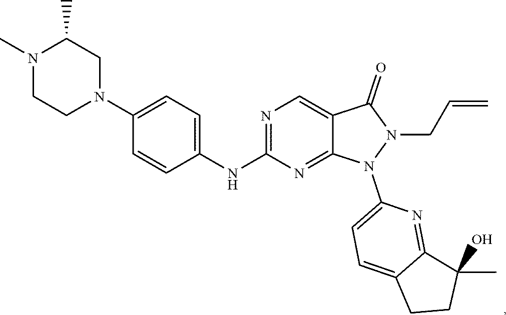 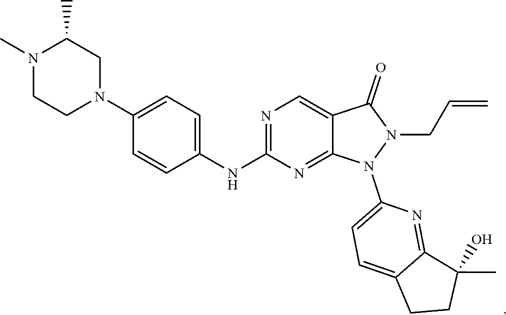 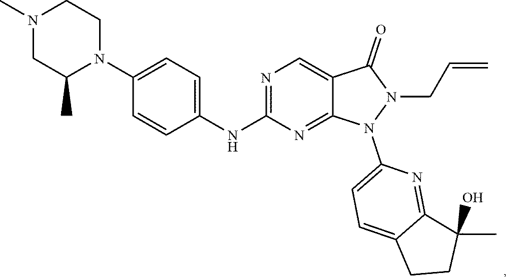 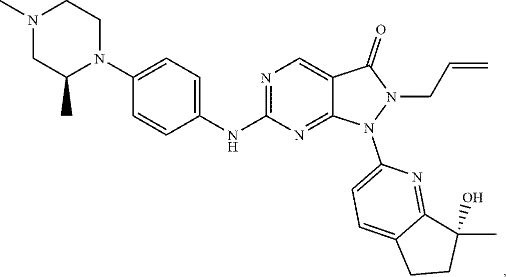 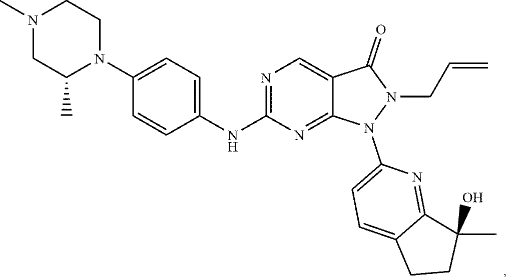 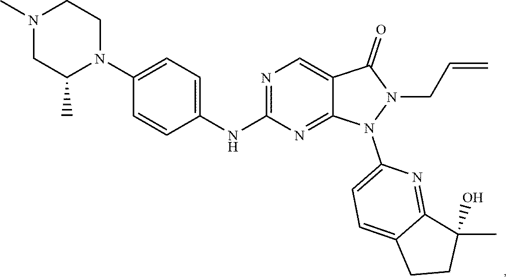 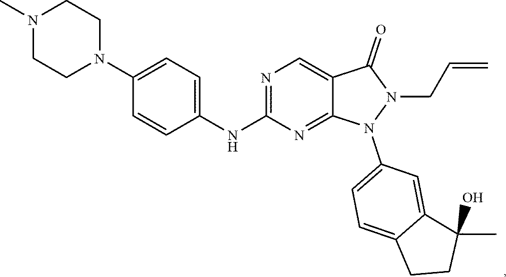 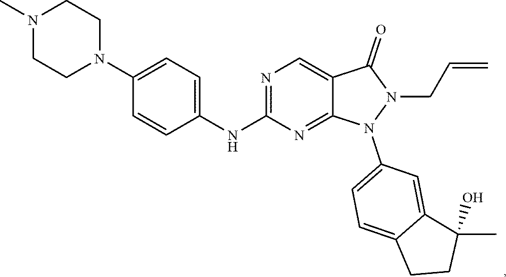 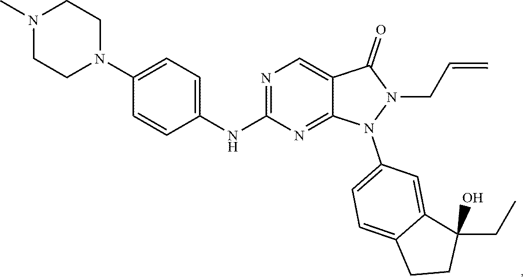 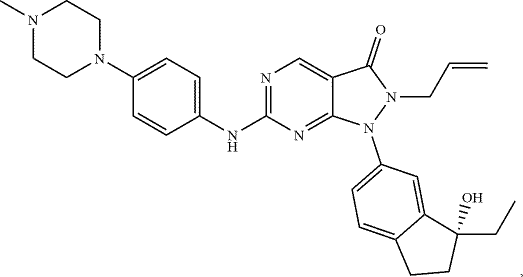 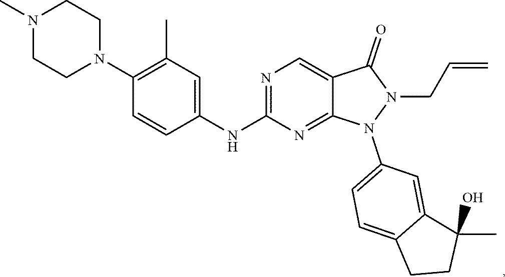 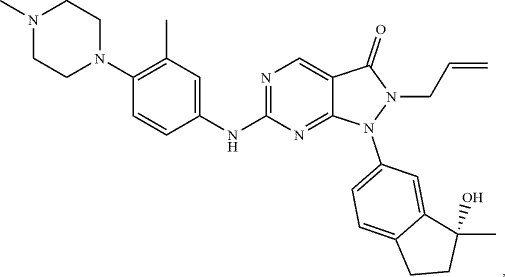 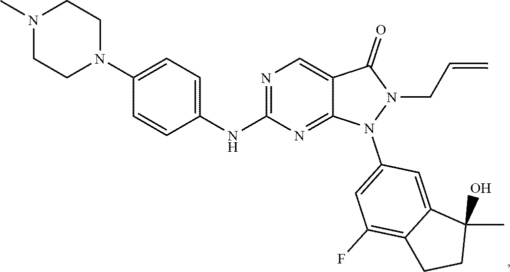 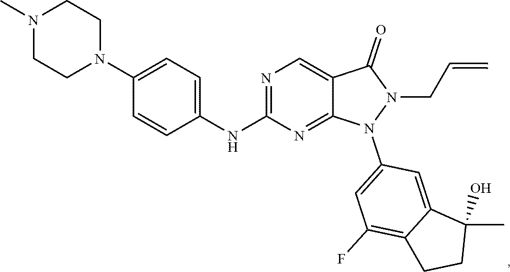 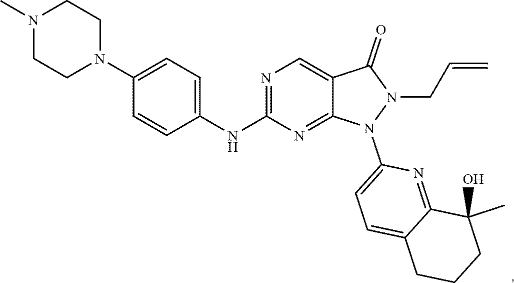 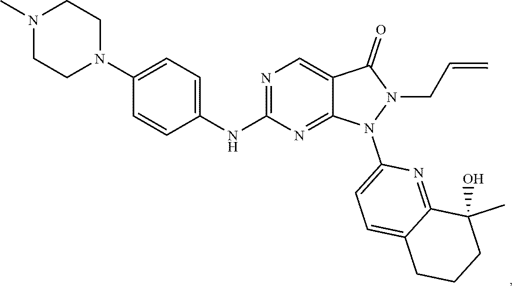 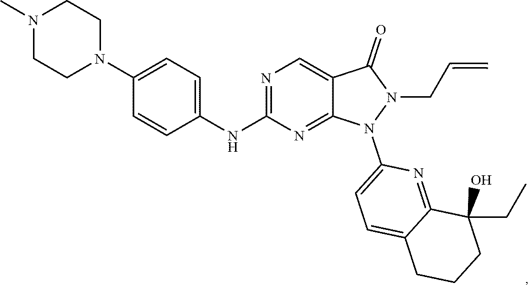 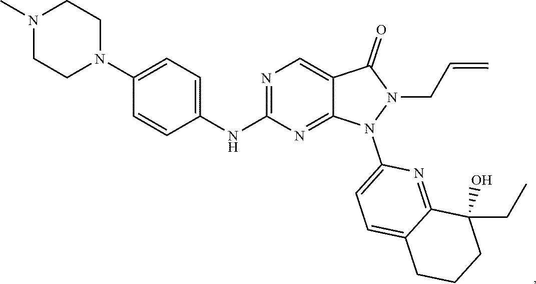 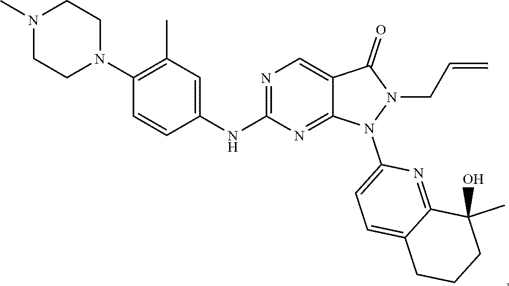 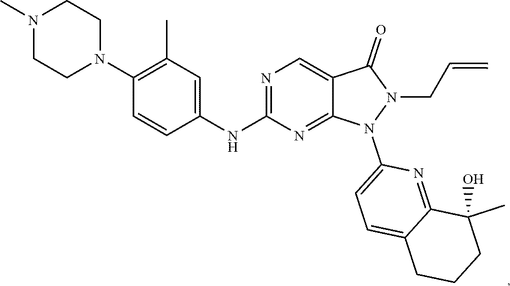 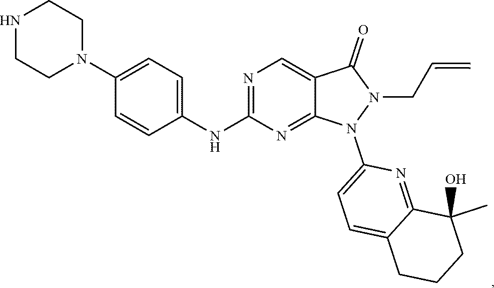 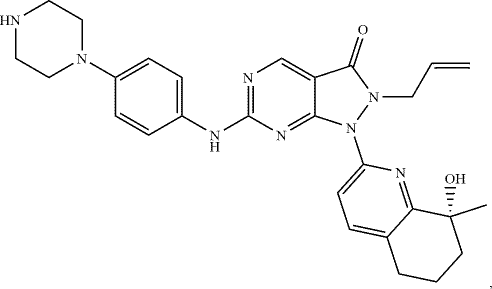 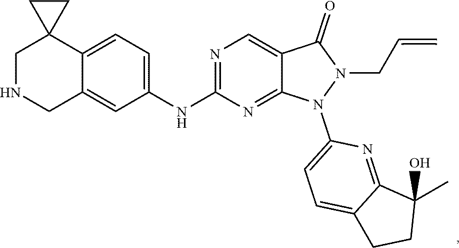 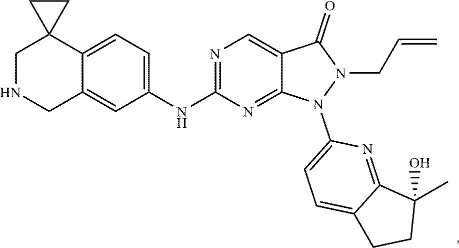 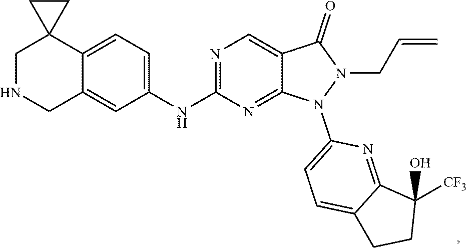 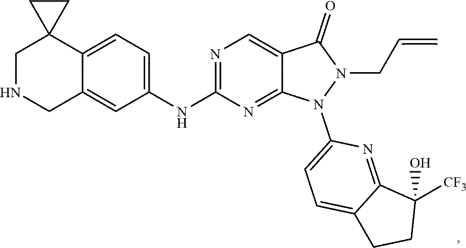 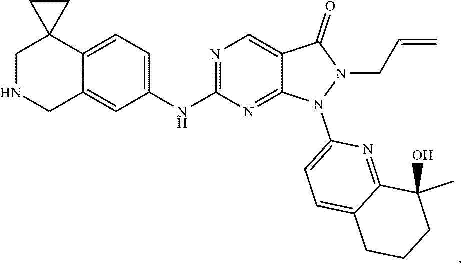 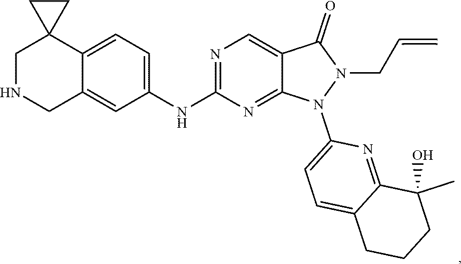 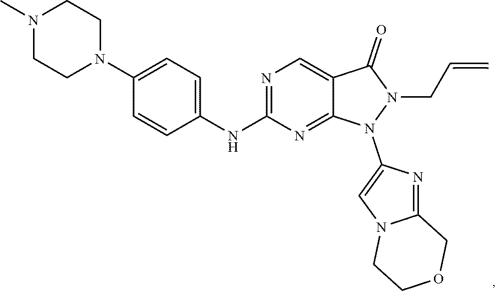 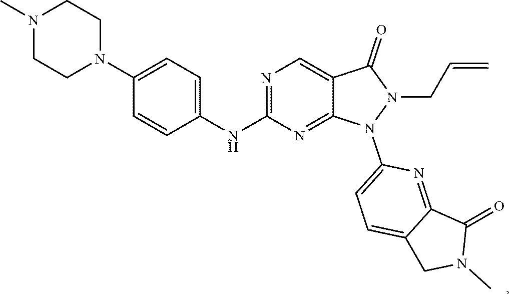 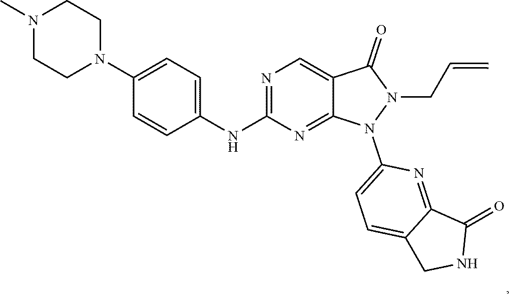 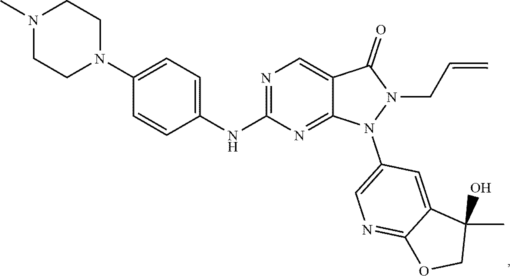 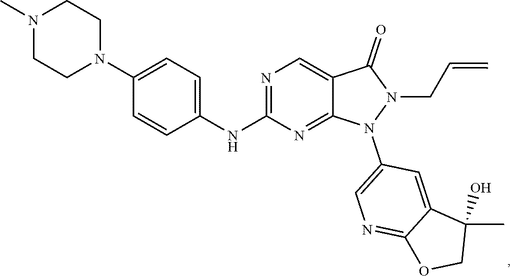 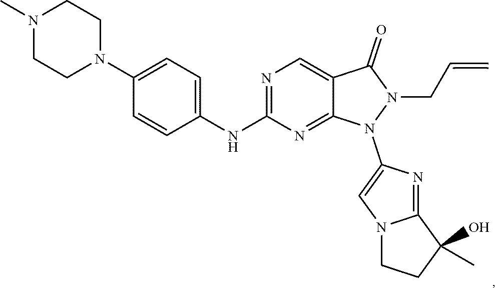 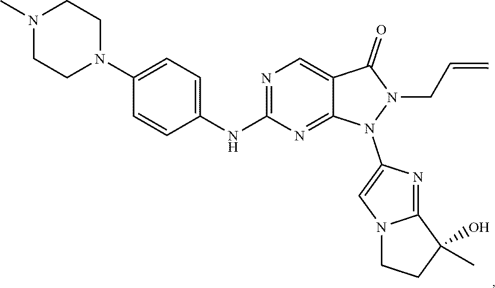 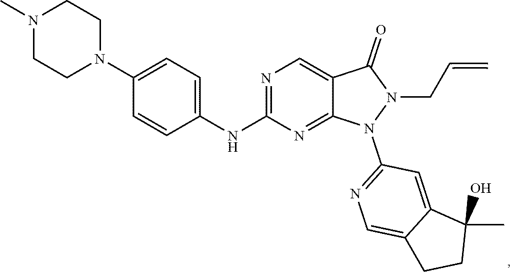 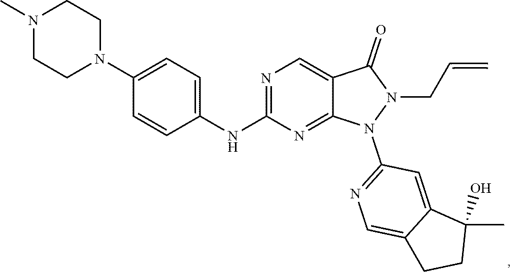 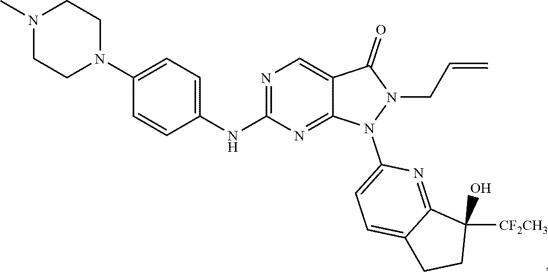 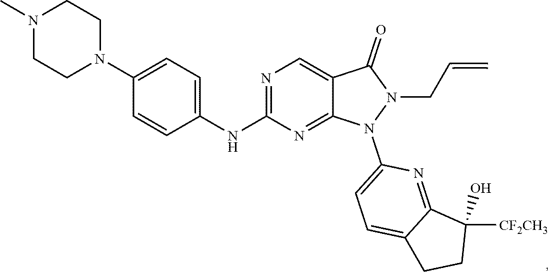 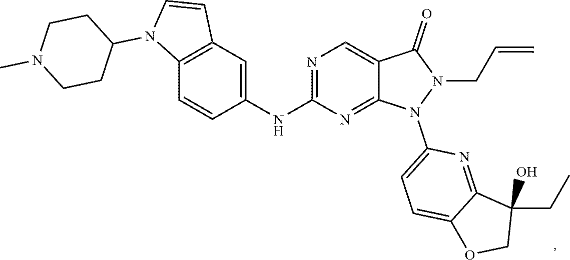 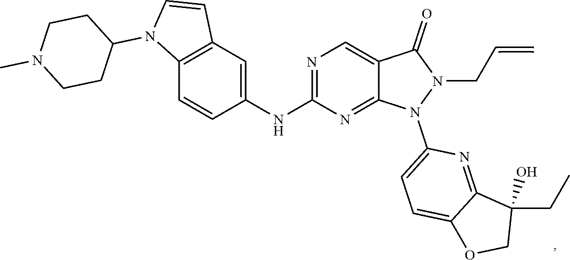 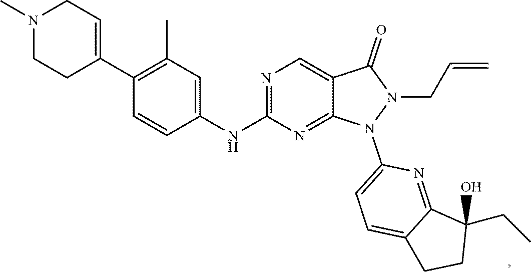 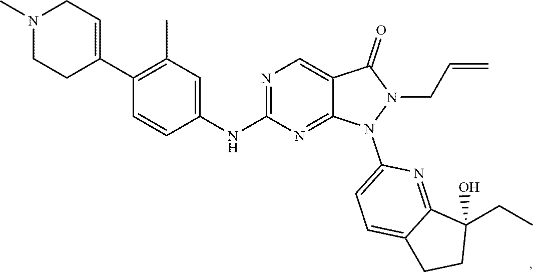 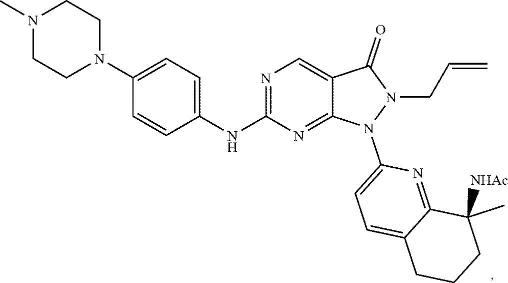 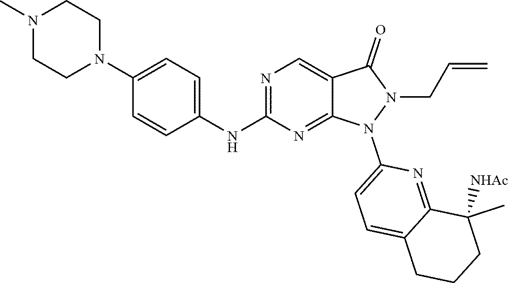
and 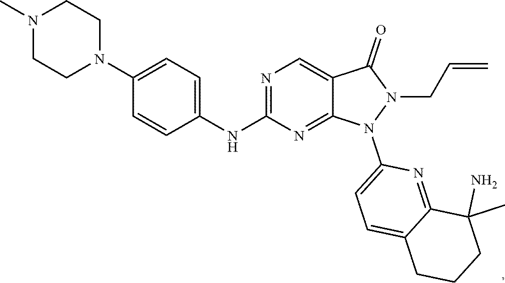
or a pharmaceutically acceptable salt of any of the foregoing.

5. The use of any one of claims 1-4, wherein the Compound (A) is 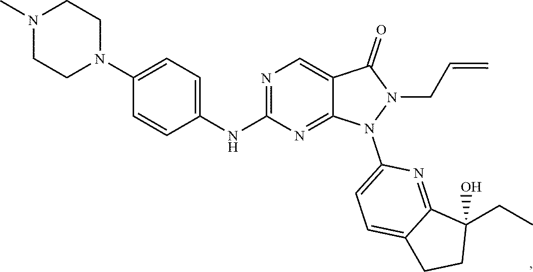
or a pharmaceutically acceptable salt thereof.

6. The use of any one of claims 1-4, wherein the Compound (A) is 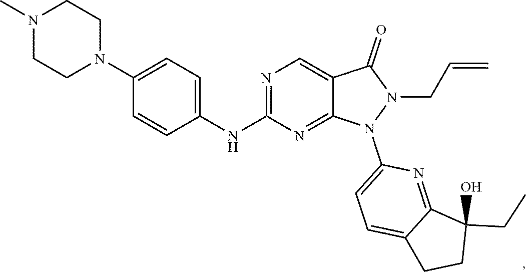
or a pharmaceutically acceptable salt thereof.

7. The use of any one of claims 1-4, wherein the compound is 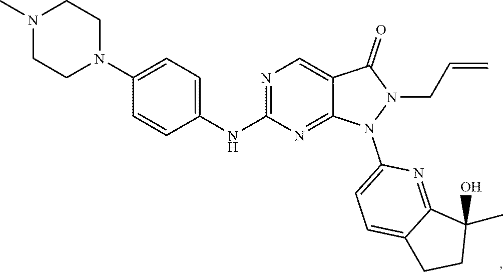
or a pharmaceutically acceptable salt thereof.

8. The use of any one of claims 1-4, wherein the compound is 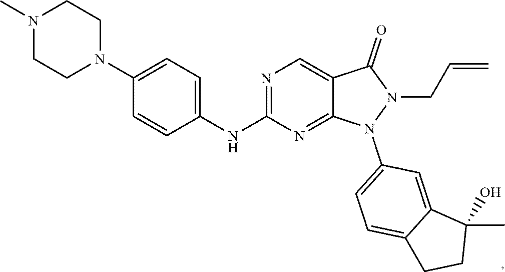
or a pharmaceutically acceptable salt thereof.

9. The use of any one of claims 1-4, wherein the compound is 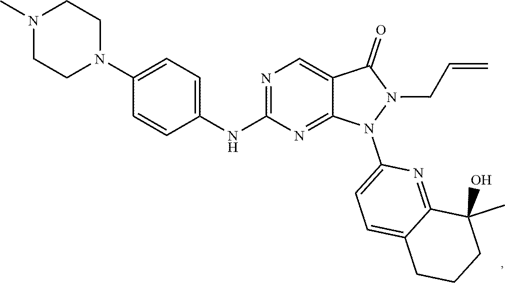
or a pharmaceutically acceptable salt thereof.

10. The use of any one of claims 1-4, wherein the compound is 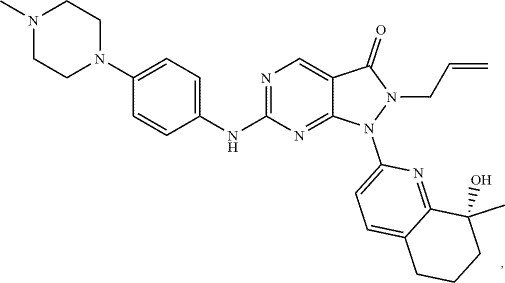
or a pharmaceutically acceptable salt thereof.

11. The use of any one of claims 1-10, wherein the disease or condition is selected from the group consisting of a brain cancer, a cervicocerebral cancer, an esophageal cancer, a thyroid cancer, a lung cancer, a breast cancer, a stomach cancer, a gallbladder/bile duct cancer, a liver cancer, a pancreatic cancer, a gastric cancer, a colon cancer, a rectal cancer, an ovarian cancer, an endometrial cancer, a choriocarcinoma, an uterus body cancer, an uterocervical cancer, a renal pelvis/ureter cancer, a bladder cancer, a prostate cancer, a penis cancer, a testicular cancer, a fetal cancer, an uterine cancer, Wilms’ cancer, a skin cancer, malignant melanoma, a neuroblastoma, an osteosarcoma, an Ewing’s tumor, a soft part sarcoma, a head and neck squamous cell carcinoma, a glioblastoma, an acute leukemia, a chronic lymphatic leukemia, a chronic myelocytic leukemia, polycythemia vera, a malignant lymphoma, multiple myeloma, a Hodgkin’s lymphoma and a non-Hodgkin’s lymphoma.

12. The use of claim 11, wherein the disease or condition is a lung cancer.

13. The use of claim 12, wherein the lung cancer is small cell lung cancer (SCLC).

14. The use of claim 12, wherein the lung cancer is non-small cell lung cancer (NSCLC).

15. The use of claim 11, wherein the disease or condition is a breast cancer.

16. The use of claim 15, wherein the breast cancer is triple negative breast cancer.

17. The use of claim 11, wherein the disease or condition is a gastric cancer.

18. The use of claim 11, wherein the disease or condition is a colon cancer.

19. The use of claim 11, wherein the disease or condition is a rectal cancer.

20. The use of claim 11, wherein the disease or condition is an ovarian cancer.

21. The use of claim 20, wherein the ovarian cancer is TP53-mutated ovarian cancer.

22. The use of claim 11, wherein the disease or condition is an uterine cancer.

23. The use of claim 11, wherein the disease or condition is an endometrial cancer.

24. The use of claim 23, wherein the endometrial cancer is uterine serous carcinoma.

25. The use of claim 11, wherein the disease or condition is a head and neck squamous cell carcinoma.

26. The use of claim 11, wherein the disease or condition is a glioblastoma.

27. The use of claim 11, wherein the disease or condition is an osteosarcoma.

28. The use of claim 1, wherein the combination is being used for a subject that has been determined to have homologous recombination deficiency (HRD) positive status.

29. The use of claim 1, wherein the combination is being used for a subject that has been determined to have homologous recombination deficiency (HRD) negative status.

30. The use of claim 28 or 29, wherein the one or more of Compound (B), or a pharmaceutically acceptable salt thereof, is a PARP inhibitor, or a pharmaceutically acceptable salt thereof.

31. The use of claim 30, wherein the PARP inhibitor, or a pharmaceutically acceptable salt thereof, is niraparib, or a pharmaceutically acceptable salt thereof.

32. The use of any one of claims 28-31, wherein the disease or condition is selected from the group consisting of an ovarian cancer, a breast cancer, a prostate cancer, a fallopian tube cancer and a primary peritoneal cancer.

33. The use of claim 32, wherein the ovarian cancer is recurrent ovarian cancer.

34. The use of claim 32, wherein the breast cancer is selected from the group consisting of triple-negative breast cancer and metastatic breast cancer.

35. The use of claim 32, wherein the prostate cancer is metastatic castration-resistant prostate cancer.

36. A compound, or a pharmaceutically acceptable salt thereof, for use in treating a disease or condition, wherein the compound is Compound (A) having the structure: 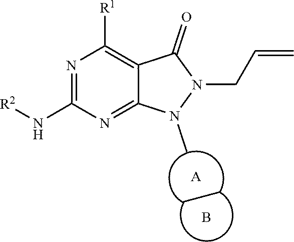
wherein:
R¹ is selected from the group consisting of hydrogen, halogen and a substituted or unsubstituted C₁-C₆ alkyl;
Ring A is selected from the group consisting of a substituted or unsubstituted phenyl and a substituted or unsubstituted 5-6 membered monocyclic heteroaryl;
Ring B is selected from the group consisting of a substituted or unsubstituted monocyclic 5-7 membered carbocyclyl and a substituted or unsubstituted 5-7 membered monocyclic heterocyclyl;
R² is selected from the group consisting of 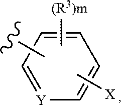 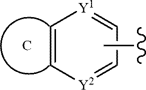
and 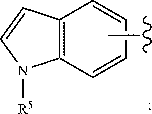
m is 0, 1, 2 or 3;
R³ is selected from the group consisting of halogen and a substituted or unsubstituted C₁-C₆ alkyl;
X is selected from the group consisting of hydrogen, halogen, hydroxy, cyano, a substituted or unsubstituted 4-6 membered monocyclic heterocyclyl, a substituted or unsubstituted amine(C₁-C₆ alkyl), a substituted or unsubstituted —NH—(CH₂)₁₋₆-amine, a mono-substituted amine, a di-substituted amine, an amino, a substituted or unsubstituted C₁-C₆ alkyl, a substituted or unsubstituted C₁-C₆ alkoxy, a substituted or unsubstituted C₃-C₆ cycloalkoxy, a substituted or unsubstituted (C₁-C₆ alkyl)acyl, a substituted or unsubstituted C-amido, a substituted or unsubstituted N-amido, a substituted or unsubstituted C-carboxy, a substituted or unsubstituted O-carboxy, a substituted or unsubstituted O-carbamyl and a substituted or unsubstituted N-carbamyl;
Y is CH or N;
Y¹ is CR^{4A} or N;
Y² is CR^{4B} or N;
Ring C is selected from the group consisting of a substituted or unsubstituted C₆-C_{lO} aryl, a substituted or unsubstituted monocyclic 5-10 membered heteroaryl, a substituted or unsubstituted monocyclic 5-7 membered carbocyclyl, a substituted or unsubstituted 5-7 membered monocyclic heterocyclyl and a substituted or unsubstituted 7-10 membered bicyclic heterocyclyl;
R^{4A} and R^{4B} are independently selected from the group consisting of hydrogen, halogen and an unsubstituted Cₗ₋₄ alkyl; and
R⁵ is a substituted or unsubstituted 5-7 membered monocyclic heterocyclyl.

37. The compound of claim 36, wherein the Compound (A) is 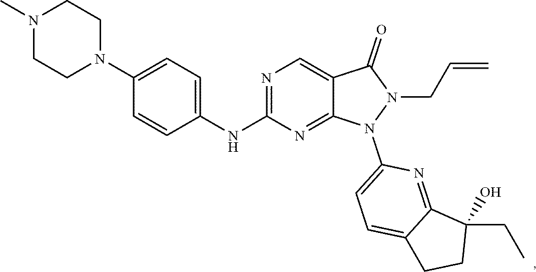
or a pharmaceutically acceptable salt thereof.

38. The compound of claim 36, wherein the Compound (A) is 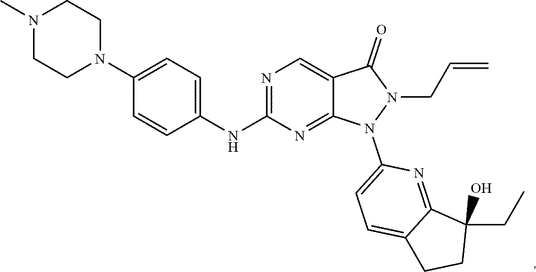
or a pharmaceutically acceptable salt thereof.

39. The compound of claim 36, wherein the compound is 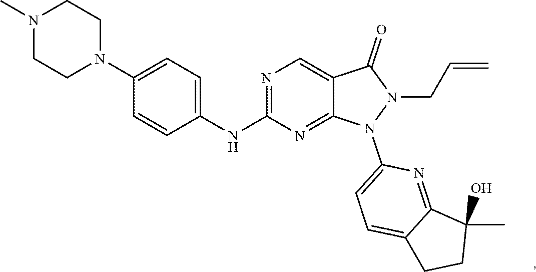
or a pharmaceutically acceptable salt thereof.

40. The compound of claim 36, wherein the compound is 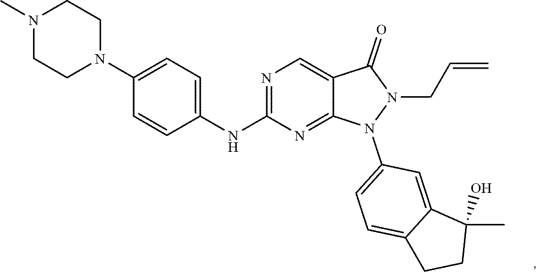
or a pharmaceutically acceptable salt thereof.

41. The compound of claim 36, wherein the compound is 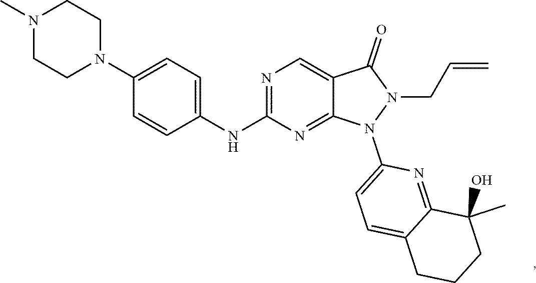
or a pharmaceutically acceptable salt thereof.

42. The compound of claim 36, wherein the compound is 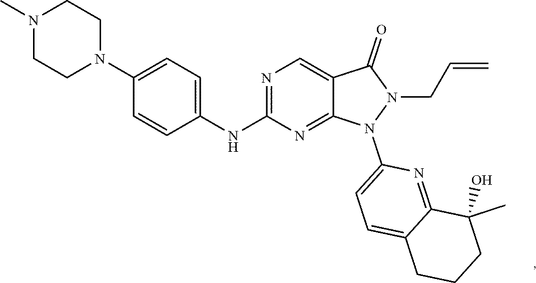
or a pharmaceutically acceptable salt thereof.

43. The compound of any one of claims 36-42, wherein the disease or condition is a breast cancer.

44. The compound of claim 43, wherein the breast cancer is triple negative breast cancer.

45. The compound of any one of claims 36-42, wherein the disease or condition is an ovarian cancer.

46. The compound of claim 45, wherein the ovarian cancer is TP53-mutated ovarian cancer.

47. The compound of any one of claims 36-42, wherein the disease or condition is an endometrial cancer.

48. The compound of claim 47, wherein the endometrial cancer is an uterine serous carcinoma.

49. The compound of any one of claims 36-42, wherein the disease or condition is a head and neck squamous cell carcinoma.

50. The compound of any one of claims 36-42, wherein the disease or condition is a glioblastoma.

51. The compound of any one of claims 36-42, wherein the disease or condition is an osteosarcoma.
